SEQUENCE LISTING (314 sequences)

SEQ ID NO: 1 - DNA, 20 bp, Artificial Sequence
  Antisense Compound
tccgtcatcgctcctcaggg

SEQ ID NO: 2 - DNA, 20 bp, Artificial Sequence
  Antisense Compound
gtgcgcgcgagcccgaaatc

SEQ ID NO: 3 - DNA, 20 bp, Artificial Sequence
  Antisense Compound
atgcattctgcccccaagga

SEQ ID NO: 4 - DNA, 4788 bp, Artificial Sequence
  H. sapiens
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 5 - DNA, 27 bp, Artificial Sequence
  PCR Primer
aggttgtgcaaattaacagtcctaact

SEQ ID NO: 6 - DNA, 23 bp, Artificial Sequence
  PCR Primer
tagtcttttgcaaccatcatcca

SEQ ID NO: 7 - DNA, 30 bp, Artificial Sequence
  PCR Probe
agcacctagtccagtgacctgctgggtaaa

SEQ ID NO: 8 - DNA, 19 bp, Artificial Sequence
  PCR Primer
gaaggtgaaggtcggagtc

SEQ ID NO: 9 - DNA, 20 bp, Artificial Sequence
  PCR Primer
gaagatggtgatgggatttc

SEQ ID NO: 10 - DNA, 20 bp, Artificial Sequence
  PCR Probe
caagcttcccgttctcagcc

SEQ ID NO: 11 - DNA, 2575 bp, Artificial Sequence
  M. musculus
ggaagttaatatttgccaatggactccaaagaatccttagctccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcacagcagcagcagcagcagcagcagccgcagccagatttatccaaagccgtttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaatcccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcacctttgacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggaatgagaccagatgtgagttctcctccgtccagctcctccacagcaacgggaccacctcccaaactctgcctggtgtgctccgatgaagcttcggtatgccattatggggtgctgacgtgtggaagctgtaaagtcttctttaaaagagcagtggaaggacagcacaattacctttgtgctggaagaaatgattgcatcattgataaaattcgaagaaaaaactgtccagcatgccgctatcgaaaatgtcttcaagctggaatgaacctggaagctcgaaaaacgaagaaaaaaattaaaggaattcagcaagccactgcaggagtctcacaagacacttctgaaaacgctaacaaaacaatagttcctgccgcgctgccacagcttacccctaccctggtgtcactgctggaggtgatcgagcctgaggtgttatatgcaggatatgacagctctgttccagactcagcatggagaattatgaccacgctcaacatgttaggtgggcgccaagtgattgccgcagtgaaatgggcaaaggcgataccaggattcagaaacttacacctggatgaccaaatgacccttctacagtactcatggatgtttctcatggcatttgccctgggttggagatcatacagacaagcaagtggaaacctgctatgctttgctcctgatctgattattaatgagcagagaatgactctaccctgcatgtatgaccaatgtaaacacatgctgtttatctccactgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccattgtcaaaagggaaggaaactccagtcagaattggcagcggttttatcaactgacaaaacttttggactccatgcatgatgtggttgaaaatctccttagctactgcttccaaacatttttggataagtccatgagtattgaattcccagagatgttagctgaaatcatcactaatcagataccaaaatactcaaatggaaatatcaaaaagcttctgtttcatcagaaatgactgccttactaagaaaggctgccttaaagaaagttgaatttatagcttttactgtacaaacttatcaacttgtcttgtagatgttttgtcgttctttttgtttgtcttgtttgttttctatacgcactacatgtggtctctagagggccaagacttggcaacagaagcagatgagccatcacttttcagtgacaggaaagcagac

SEQ ID NO: 12 - DNA, 23 bp, Artificial Sequence
  PCR Primer
gacatcttgcaggatttggagtt

SEQ ID NO: 13 - DNA, 23 bp, Artificial Sequence
  PCR Primer
aacaggtctgacctccaaggact

SEQ ID NO: 14 - DNA, 27 bp, Artificial Sequence
  PCR Probe
cgggtccccaggtaaagagacaaacga

SEQ ID NO: 15 - DNA, 20 bp, Artificial Sequence
  PCR Primer
ggcaaattcaacggcacagt

SEQ ID NO: 16 - DNA, 20 bp, Artificial Sequence
  PCR Primer
gggtctcgctcctggaagat

SEQ ID NO: 17 - DNA, 27 bp, Artificial Sequence
  PCR Probe
aaggccgagaatgggaagcttgtcatc

SEQ ID NO: 18 - DNA, 6322 bp, Artificial Sequence
  R. norvegicus
  misc_feature (1)...(6322): n = A,T,C or G
gacgctgcgggggtgggggacctncggcggcacggagtccccccccgggctcacattaatatttgccaatggactccaaagaatccttagctccccctggtagagacgaagtccctggcagtttgcttggccaagggagggggagcgtaatggacttttataaaagcctgaggggaggagctacagtcaaggtttctgcatcttcgccctcagtggctgctgcttctcaggcagattccaagcagcagaggattctccttgatttctcgaaaggctccacaagcaatgtgcagcagcgacagcagcagcagcagcagcagcagcagcagcagcagcagcagcagcagcagcagcagccaggcttatccaaagccgtttcactgtccatggggctgtatatgggagagacagaaacaaaagtgatggggaatgacttgggctacccacagcagggccaacttggcctttcctctggggaaacagactttcggcttctggaagaaagcattgcaaacctcaataggtcgaccagcgttccagagaaccccaagagttcaacgtctgcaactgggtgtgctaccccgacagagaaggagtttcccaaaactcactcggatgcatcttcagaacagcaaaatcgaaaaagccagaccggcaccaacggaggcagtgtgaaattgtatcccacagaccaaagcacctttgacctcttgaaggatttggagttttccgctgggtccccaagtaaagacacaaacgagagtccctggagatcagatctgttgatagatgaaaacttgctttctcctttggcgggagaagatgatccattccttctcgaagggaacacgaatgaggattgtaagcctcttattttaccggacactaaacctaaaattaaggatactggagatacaatcttatcaagtcccagcagtgtggcactaccccaagtgaaaacagaaaaagatgatttcattgaactttgcacccccggggtaattaagcaagagaaactgggcccagtttattgtcaggcaagcttttctgggacaaatataattggtaataaaatgtctgccatttctgttcatggtgtgagtacctctggaggacagatgtaccactatgacatgaatacagcatccctttctcagcagcaggatcagaagcctgtttttaatgtcattccaccaattcctgttggttctgaaaactggaataggtgccaaggctccggagaggacagcctgacttccttgggggctctgaacttcccaggccggtcagtgttttctaatgggtactcaagccctggaatgagaccagatgtaagctctcctccatccagctcgtcagcagccacgggaccacctcccaagctctgcctggtgtgctccgatgaagcttcaggatgtcattacggggtgctgacatgtggaagctgcaaagtattctttaaaagagcagtggaaggacagcacaattacctttgtgctggaagaaacgattgcatcattgataaaattcgaaggaaaaactgcccagcatgccgctatcggaaatgtcttcaggctggaatgaaccttgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgatactaggcttgagaaacttacacctcgatgaccaaatgaccctgctacagtactcatggatgtttctcatggcatttgccttgggttggagatcatacagacaatcaagcggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgactgccttactaagaaaggttgccttaaagaaagttgaatttatagcttttactgtacaaacttatcaatttgtcttgtagatgttttgttgttctttttgtttctgtcttgttttgttttaaacacgcagtacatgtggtttatagagggccaagacttggcgacagaagcagttgagtcaacactctgaagtgatgacacagcacacagtgaagtgtattgttggtgtatcacagaaactaacagttacgtggaggcatggccactgtcagagagggaccgcacctaaaccaccgtgcccaagtccatgtggttcaactttctgactcagaactttacagttggctgggtaaaactttctagactttctgttggtgtatttttcccatgtatagttaggatggtattttgatttatgcatgcaaacctgaaaaaagtttacaagtgtatatcagaaaagggaagttgtgccttttatagctattactgtctggttttaacaatttcctttatattcagtgaactatgcttgctcgtttctcttcaataatttttgtattccagttattgtacagctgtttaagatgggcagctgcttcacagctttcctagacgctaacattaatttccgtgtgaaaatgggtcggtgcttctaccctgttggcaccagctatcagaagaccacagaaattgactcagatctccagtattcttgttaaaaagctcttactctgtatatatctgcttccatggagaattacataggctgagcagattacataggctgagcagattaaccgtcctaactggtgtagagcacctagtccagtgaccttctgggtaaaccgtggatgatggttacagaagactggtgggaaaacagtaactaccaaaaggcccctttccatctaatgcaccatctcttcaatggggagatagcaaccaagcccgtaaatcagctctttcaggaccttctggagtggtttgcataacattttaaaatgtattattccagatagccagctctgataaagccgagagattgtttaatcagaccaagtaacttctctcattaaacttacccccaactaaatcgctaatacagcaagaatggctagacacccattttcacatctcacccgcaccgattggtctagctctcatggtggtcaggagaatcagctactgatttttgttacttagaatnttcaggactcgcatttntccnnctacacatccctacatgtgccatagaatttaacacaagtcctgtgaacttcttcacattgagaattatcattttaaacaaaacagaagcagtagtagccctttctntgtgcaccttaccnnctttctntgactcaaagcttaatatgcttactaagccacaagaaatcngatttcnacttaaaggcgccaaattatttgtgtaatagaaaaactgaaaatctaatattaaaaatatgaaacttctaatatatttttatatttagttatagtttcgatatatatcatatcggtattcactgatcttgggaaagggaaagggctactgcagctttacatgcaatttattaactgactgtaaaatagctgtatagtaataagaatgacttttagtgagattgctttatcatgacatgttatatatttttcgtaggggtcaaagaaatattgatggatatgatagcctatatgatttaatngtatataaaagcatncaaacaggccttaacgcgtcttggaaannaaaatacctttgttctaagctagggaagggagcnggagannggccccgtgtgtatnggaggttccgaggctcggatnnaagagatcnanaggggatctaattccntacctccatctaattacctcaccacccatgatcctgtcagtgnaggnnnggttattaaatcccccgttatactaatataaatagganagaagggtggcgctcacgtctgttccaggcgccgcagtagcagggttattttccatgcagcctcccgacaaggttagcagagggaggctttggcaagtttggcgtggcgtgcatagaggcaccagcaacatgtaaacctaaagagcccataggaagccaagaatacactaatcctccccacccttcaatagtccatttccaagtaagatgaggacatgcttatgttttctttgaatgcttttagaatgttgttattttcagtattttgcagaaattatttaataaaaaagtataatttgaattctctctaaaagggattgttcagtttgtaatggtttaaattggtctcaaagtactttaagataattgtaacccagctggatgtgaaatttatggtgcctaagaaataccacttgaatattatcaagacagtgttaagttttaaaatgagcttctcaaaaatagattattgtacatttatggaatgttatatggttaaacccaaaaaagcacatcacacataaatctgctttcagcttggctttcaaaaatagagctccaaaaacgaaaaaggagaagaaaaagtatatatatgcgttgttattaacagaaggcaacagacattcataaaactactaccgaagctttccttgaagcgtataaagagccatgctcctttagtatgtggggaagaagagagccgtcatagtttcgagtacagagagaagatgcggtactgtctccgtgtgtggcttcataccgttcctaactatttaggtttataataacttcagtgagactcggtgacatgcctgtatgactcatgaccgatcttgaaagatatctttaattactggtaggacaaaagggacactctggttattttaggccttggcttgggatactgtatatccagaagaaaggagacaggaaacttggggaagggaagggaacctaggaagcactgccttctgtaggaaagaacacaccaataagtgagagtacccaaagggacaaggccacacagtgtggggtctaaggatgagtcagggtgagctctggtgggcatggagaagccagcaactccagtgctacagagcagggcagggcagggatgggacaagatggatgcggatcccagtcccagtagtttgctccctcttatttaccatgggatgaaccatggagtattgatctgtcagcactcaaggatcatggagcttgagattccggttggtcaccccaacggtaagctgagattgaatgtgtttcttatgtgccggtttcagtgttagaaggcgaaacagagtgtacagaagacactgcaaaccggtcagatgaaagtcttctcattcccaaactattttcagtcagcctgctctatcaggactggtgaccagctgctaggacagggtcggcgcttctgtctagaatatgcctgaaaggattttattttctgataaatggctgtatgaaaataccctcctcaataacctgcttaactacatagagatttcagtgtgtcaatattctattttgtatattaaacaaaggctatataatggggacaaatctatattatactgtgtatggcattattaagaagcttttnnannattttttatcacagtaatttttaaatgtgtaaaaaattaaaaattagtgantccngtttaaaaataaaagttgtagttttttattcatgctgaataacctgtagtttaaaaatccgtctttctacctacanagtgaaatgtcagacngtaaaattttgtgtggaaatgtttaacttttatttttctttaaatttgctgtcttggtattaccaaaccacacattgtactgaattggcagtaaatgttagtcagccatttacagcaatgccaaatatggataaacatcataataaaatatctgctttttc

SEQ ID NO: 19 - DNA, 25 bp, Artificial Sequence
  PCR Primer
aaacaatagttcctgcagcattacc

SEQ ID NO: 20 - DNA, 23 bp, Artificial Sequence
  PCR Primer
catacaacacctcgggttcaatc

SEQ ID NO: 21 - DNA, 23 bp, Artificial Sequence
  PCR Probe
acccctaccttggtgtcactgct

SEQ ID NO: 22 - DNA, 23 bp, Artificial Sequence
  PCR Primer
tgttctagagacagccgcatctt

SEQ ID NO: 23 - DNA, 21 bp, Artificial Sequence
  PCR Primer
caccgaccttcaccatcttgt

SEQ ID NO: 24 - DNA, 24 bp, Artificial Sequence
  PCR Probe
ttgtgcagtgccagcctcgtctca

SEQ ID NO: 25 - DNA, 32767 bp, Artificial Sequence
  H. sapeins
aagcttatggtttatgggtgttacattcaagacatttgtaggacacattctaaaatgccatccaatttcaggctctttccagcagaaactgtggaatatttttccgttcattcagcatttacttagtgcctgctctgccaggaattgaagagaaagcccaaagacaggcagaccttacctgagaggtagtgaactgaccaggatgactgtgggcagtagacttgtttcccaaactagcctcaccatttctgtatttgcatatacgaggaaaggattagatatagggattcatgtcagcatacaccccagggacatttgtttttagtgaaaggtgccagtcttcatccctgtacccagtacacaaaccacgaagaagtatgctcccgtcattgtcaaagaatcatagaattccaaatggagctagttttgatatccagatctcacttcatatgaggaaactaggtccagtattgtgagtaagaattaggactcttcagattccctgggtatgaatctgactaacaactgtgtgaacttgaccaaattcataaccctgtaaactctgtttcctcacttttaaaatgggcacaacaaagtgatgcatgtaaactgcatagcacagtgtctggcacttaaaaagcactcctgaagttatttttagtgatgtgttttaagattagacaactccttaatgccaaaggtttttacttgagaactctgtctgtgtgccatactacacgctgttcataagataagcctttttcattaattgatctcaaactggcttcattatgatcttaactttatttcagttttatttttaaaatttatttttaatttttatgggtatatagtaggcatatatatttatggggtacaggtcatgttttaatgcaagcatgcaattgtgggggtgatatataattgactggggtgagatatctcattgtagttttgatttgcatttctctgatgattaaggatgttgaacatttcttcatacacctgttggccatttgtatgtcttttgagaaatgtctattcagatcttttgtccattttttaagttggattgtttgattttttcctgttgtctgaactctttatatattctagttattaatcccttctcagatgggtagcttgcaaatattttcttccattttgtgggttgcttctttgttgtttccgttgctgtgcagaagttttttagcttgatgtgatcccatttgtccatttttgcattggttgcctgtgcatttgaggtattactaaagaaatctttgcccataccagtgtcctggagagcttcccaaatgttttcttttagtatcctagtttcaggtcttagatttagggctttagtccatttttatttgatttttatatgtggtgagagataggggtctagtttcattctgcctatggatatccagttttcccagcaccatttattgaagagactgtcctttccctagtgtatgttcttggcacctttgctgaaaatgagttcactgtaggtgtatgaatttgtttctgggttctctaggtctgtgtatctgtttttatgctagaactatgttgtttgggttattatagttttgtagcataatttgaagtcagataatgtaattcctccagttttattttttttgttcaggatggctttggctattccggggcttttgtggttccatataaatcctatgattttttttttctatttctgtgaagaatgtcattgatatttattaataaagattgcattgaatctgtagattgctttgggtagtatggacattttaacaatattgattcttccaatccatgagcatggactatctttctttttttgtgtgtcctcttcaatatttttcctcagtgttttattgttttcattgtagagctctttcacttctttcgttgagtttattcctaggtgttttattttatctgtagctattgtaaatgagattactttctgatttcttttttagattgtcctctgttggcatctagaaatgccacagatttttgtatgttgattttgtatcctgtaactgtactgaatttatctgttctaatatttttttggtggagtctttaggcttttccaataagatcatacagtctgcaaacaagaataatttgacttcttccattccattttggattccctttatatctttctcttgtctgattactctaggtaggtcttccagtacttccagttgaataacagtgggcactcttgtcttgttgtagatcttagaagaaaggctttcagtttttccccattcagtatgatactagctgtcagtttgttgcagatggcataactttcaaactaattgattatagttaggaagtggatactttaacttgtggtaccattatcagatttatatttcggccataagcttgaagaggagctgaaaaatgcatatgtgatgcatatgcttcctatttggctctcttctcccacccccctgccctataatccacacaagttcctctctcagtcactcatcaactacttgaacctctgaggaacttggggttaaggtaaattagaataaaactgtctgaagaagagcaagcctttcatgtcttgagaaattcttggggttttagaaataacttcattgctttttttctccagttactttggcttcttcttaaagagaatactaacactttgaacgtcataatactaaggttctgcctcttcaaataaagactttaaaaaaaaatggtttttgtatgattcagtgtgaattaaatcccacagtgtaaaggactttactttcttaatgtagattttcaaatacacaattactgatgtttataagtagatttattacaccaaagcacctagcaaattcttgaatggatcaggtcttatttttcagtcttactttgcaaatttaagccaaataattaaggatttgttaaatatttgtcttaatatcaagcttttgcatatcggggccctcttttataagctttataagcaatcttttgttttctctgcttgctcaaagtagctatgtttgttgtatctgttagtatttgctctataacaaacatactgggtgccttcccacttagatttggcaattatcactcctgtaaatgagatattacataagataggaaaaagaacagtatctttccaagaagaatagtatccttccatattaacagtttagagctgactgcttttaaaatttagtggctttaaaataacaaccatttattattcttcatgagtctacaaatgaggtgggcagttctgctgatctggccaagctgaacttatctcagctgggcacattcagcgtatctgctgtcagttggctggttggctgtagcaatgaatggtgaaagtaggctgcccttaactttttcacacagtagcattagagttacaaaagaaccagcagaaccatgcaaaactctttaagacctaggcttggaacaactatatttctaccacattctattggtcaaagcaaatcacggggctagtctagattcaagtgggtggaggagctgcaattacactgcaaaggagtgtgactgtagggagaggtgtttttttatttttatttttttgcgatttgtcacagtagttgtaggaatcaggtgtatttaaaattctgatccttctgtgatatccgaattgttcatgaaccttgcctctggtggaaaggcagaatcattgtgacagaaggataaaatcttggaatttagagactaacaaaggttcagattccagctccatcacttatttctgcaatcctgcagaagttaatcttcctgataggcattcagtaatgattgattcacctgaacctcagattctttatgtattttaaagaaagggctaggtaaatgcaaagcacttatgtaactgcttttattattgcaaacctggctcccacactccattcaaggtgtaagactcagtgtcttccttgaattaaaaaggaagagaaagtgtgttagggaaaggaagagaaatatttgactaattgtggccccaataaagtgaccactcactgggggtattttcctgtaagaaaagaatggttgaggctcagagttaagagatacaaatccaaaagtctccttggggtaggattccctgtgattcatgggttgagaggtgtaacattagacacagtcccagtctagattttttttttaaagaattgtggtccatcctatacacactgggtgccttaatactatatgtggcaattatcactcctataaatcaggttttacataagataggaaaaagaacagtatcattccacattaacaattgaaagatgactgcttttaaaaaattaaaagggccatatagaaataaaatcacataaatttcttgtgttaaacatagttgtcatattggatgaggactaaacacctaaattcatccaactagtagtaatagaaaagatgaaacacacacacagtaaaactagattaatttaatttatacaaagggccagatatctcagaattcagacagtcagagatgttgactagagttaatgcctcttttaggagaggtaccaggtaagtgttctcaaagaactggaaactgagaccaccacctctggcattatctgtttgtgaacacaagcaagtctgaatttttccgcaccatagctacctttcatgtaagcttcttttcttagaagaaaagaaggtaacatttgggtgtaattttttattaagggtgaaatttagtgtagagagtaaaggcatttggcatagaagcccttagttttttttgtttttaagttgaactgccagcctttatggattgcagtcttcgctgttttgattgacatttcccaattcattttgtattatttatttttttaagagacagggtctcactctgttacccaggctggagtgcaatggggcaaacttggatcactgcagccttgaactcctgggctcaagcaatcctcccacctcagcctcccaagtagcttggactataggtgtgcaccaccatccttggctaattttttaaatcttttgtagagacagggtagtgctctgttgcccaggctggtctcacattcctggcctcagttgatgctctggtctcagccttccaaaatgctgggattacaagtgtgagccactgcacctggcccccaatttcatcctttacaaagactactttcaaccataaatcaacggaaacttcagctccctcagacatatttgggatccaaggatattttcccaaatgattaatgctaatttcatatcaatacatttttgcaaaacctacaaaaatggactagtaaagaaagactcttaatttgggaaagacagttacttggagagaagagaaacttaagaggcaggtcgagttcagtgttcagaaatgagaggatcataaagagatagccataaaaatgtttctccctatattgcctgctgatagggtgtatcagtgaaggtcttactaaggaccttgtaccttttcagcgctgcactgcgtgctcatagggaggaaagataaatcatgtgttttttctgacctcaaaggagcctgtatctggctagagagacatgatgcagacacatgaaataattaagaaacaattaactgtagcaggtgctgaagaatataccaggaggtcagagaatggtagagctagtgtgggcgaaggtatagcccagagcatcatcagatgattcttccttatgcaaattcacatctcctctgggtcaagtatcatcctggcatgcagcagctccataggtaatgccctaaggctagcctgaggcaagttgcaaaagccatcatattgagtcatggcctttttttgtgtggggggaggggaatggcatccccttcctgtctgccaaatcaaggaatacagtgccctcctaaacctgctttgttttagtggattgttaaaaagaagtgaatgaatttatgcttcattagggaaaggttacagtggaatactgaggagtaaggggtatttctatttaacaaatgacataacttgaaggaatgaaatcataaggatggaatttcaggcattaataaaaagctgatgagagatactttgagacaaaagagccttcccagtgtaaccgagatcacagcacctacttcacatacacaggaaaccagtcctatctgtctctcccatagagcagtagctgccttgtttttcctccctcctccatcattcattctaaatctccagtcctccaccgcaccttatccaaaccctgatacccttaagtcacagatggtgaatcagtcaaaagtagtattaaaaactagtggtacacagctacacctggaatgcagtaagaaaaatacggatttctgtacatcatcttccctccctgctcttacccccatttaagagttacagggtcagaacccaagagtgtgagtttttgaaagtccctaaaaattttggatgatcacctacatttagaaccactgcactaagaaggacaacaaatatgccaataaattctgttgccaaggaggtgattatgcaagctggaaccctgataacatgaggagaatcccacaatagccaaatagtccatgtactagttacattataataaagccaaaagcagcaggcctacctgactttctcctgaggtctatcatgagcttagagagaaggaacgtggacatacagaggtagctctagatggagaagggcactaggtgtcatggaaagaatcatgtgcaagaagtaaagaggtgctctgaatgtcctagccctgcttaggtgtctgtgtcctcacatgagaatttatccacagttctttcccgctgtaacaatctttggttccaactgcatttgtgagacagcaaaaagctatggtccagtctccttccattgtatcatctcatcaatgtatttctcccactacccttgtgtgaaatacaaacttttttggcttattgtgattatgcaaggtgtatgccaacttttttttttctccacatctttcagctttctgatgggtaaaaattttccttattttgctttagaaaaattctcattggcatagatctaatttcagggagcctcccttgaaagctaaataacattgagaattcatgaaaatataatgtagagcattatgcctgttagcatattagtttaaatagaagtggttcatgaaaatttttgaaatgccagaccctgtcctgtgttttgtattctcccaaatactcatccagatactgttcagaatgtaacatgattattttgaaataaagattttcccctagtttttaaaaaagttactttatacattaacccttatgttcctctttgatcaatttttccagtagtgtaaacagtcttcagggaagtagatttcttacagaaattgtcaagtggctctctgctgttagcatggttactaatcttttggttacttttcatattttttatactttctggaagtggacaacttacttgtaaataaaagtgcataatttgtattaaaaatttttagtaacaatctaatttgtaaaatagatgtgagcagcatgaatgtgtgtgatatgcgtacatacgaattatgtctcttaaaaatgtatcacagacatctttccgtgtccaaacaaatctacctcattctttctaatagccatatgggtataccataatatatttaactaggcccctattaaaagaattttgactcttttgtagctactatagtgttgcagtgtgtatctgtgtatgtatctttgtgtgtgtatctttgtacgagtgtacatatattttccccttggctatttcagatttttttttaggtttaaatcttaggaaaggttttgaaattgtcttaagtattttcagaagcattaaatcatggtttttttacatttttcttttagaagttttatgtcatctctatgagtagctttcagtaatttgttctgcataaaattcccgaaaacttccatttaaaaataggtggcatgactagactttctcagccgaaagagtgaggtcccaggaaggattttggagaagctgtgttcaaatatagctgctgacctgatgtctgcctagagtctggcaaggtgatgtgttgaatctagtgtctgcctgcatgccagcatccctttactgatgagatttgtggttttcatcacttcatggtaatcatcccaagttataagatggagtctctagaaaatcagtagagtatgaaggcccaagtaaaatacatgtgagtgcatgtatgtgtgcatacaaattacttctcttaaaaacgtatcctgggcatttaaagaatgaggacctccgaaggattttgtggaagctgtgttcaagtacagctgctgagcgtatgtcagcctggagcctggcaaggtgaagtgttgaatctagtgtctttttgactcactgttttttttgactcactgtgctttgaagcccttgtcatttgggctcataaaatagatttctgtatactgtctctcctccctgccctcgcccccatttaaaagtatagtggcagaacccaagaatcagagttactaaaaactctctagaaaatttggatgatcacccacctgatcatgtcttttttactcactatgttttttttttttttgagacagagtctcgctctgtcgcccaggctggagtgcagtggcatgatcttggctcactgcaagctccgcctccctggttcacgccattctcctgcctcagcctcccatgtagctgggactacagggcctgccaccgcgcccggctaattttttgtatttttagtagagtcggggtttcactgtgttagccaggatggtcccgatctcctgacctcgtgatccacccgcctcggcctcccaaagtgctgggattacaggcgtgagccaccacacccggccctttactcactatgtttttaagcccttgttttcatttgctccactgtaaaacattccccaagccaatctggagctgaggcaaatttttaacaatttaaaatctggggaatataaatattggataatgatcatcctgaaaaaacaatgaaggtagtagcataatactttatatatcaataaaatggcaaaataagacagttgttgaaggacagaaagagtaactgaagttaggagcttatcttaacacattttttgtgtcataccataggcatcatattttttaaattttttttatttcatacacataggaaaatatatgtgtgtaagaaataataaacacctctttgtacctaccacccaacttaaggaacagctcattgctattccctttggtgctcgctggatgccctttcccagtcacatccccctcccttcccatctgcaggactatactagtaaattttgtattttttgcattattttgctttgttttatgattttactacctatctacatatccctaaataatacattatttagtttcatatgttttaactttatgttgtggaatcacattaaatgtagtcttttttttttatattatactttaagttctagggtacatgtgcacaacgtgcaggtttgttacgtaggtatacatgcgccatgttggtttgctgcacccatcaactcgtcatttacactgggtatttctcctaatgctatccctcccctagccccccaccccccgataaatgtagtctttataacttgtttttttaactcaacattgtttgtaagattcatccatgtaagctgaagcttttttatagagatctttgttaagccttttaatgaatacagtacatacatttctctgttcccctgttagtggacacttggattgtttccagagttttgctgttttgaacaacgctgctgtgaaaatgtctcctgaaacacatttataagagtttttttttccccaagggaattatacctagaaattgaataactagatcacaaggcatacacatctacaacttctgctaggtaatgccaaattgtttccaaggagcgttagaagtgttctcatcaacttttactagtgctagtcttttacatttgtggcagtatggtgggtgtgaaatatttatgtttagtttttcttggtgccatttaataatttttataaaaaatatttagaagtcaaggcagttttttgtttttgtttttattttttgcttgttttgttttaatgcagacattgagattacgacttggaataaacattggttgcaaagttcctaaaaggaaaactttttttggtattctggagcttttctggtactgaataaaataagtatgttaaattatgcatgtgtagtttagaagtcagagcaataattgtgattgttgaacagaatggcagtaaaaagtttctaaacgattgtactgtacaagggacacttgttgtgggtcagttttagcctccccaacttttatgttaaaagttgcaacaaggtttaagggcttatgtttgataggccagatggtgaccagctgtgataaaacacagggaacccttgcaaaggatttcaaaatttatgcagtagtccgccttatctgcagttttgctttccaaggtttcagttacccgcagtcaactgtgttctgaaaatattaagtgaaaaattacagaaataaagaatcgaagagttttaaattttatgcttcccacccatcccacctgggatgtgaatcattcctttgttcagcatctccatgctgtaggtgctgcctgccccttagtcacttggtagccatccaggttatcagattgactcttctagtattacaacacttggcttcaagtaatccttattttacttcatagtggccccaaagtgcaggagtggtgatcctggcaattcagatatgtcaaagagaagctgtaaattgcttcccttaagtgaaagatgaaaattctagacttatatataaagaaaagaaatcatatgctgagactgctaagatctatgataagaatgaatcttttatacatgaaattgtgaagaatgaaaaagaaatgcgtgctggttttgctgtcatatctcagactgcaaaagtttgcagccaatgtgtatgataagtgcttagttaaaaggaaaaaggcatttaaggtaagtatatatagtgtttggtactacctgtgatttcaggcatccattgggggtctcctgagtataaggggagactactcttttagtgttaaatgaacactaaggaacagagatggggaagaggttggagaagattagttcagcagtttgagtataggtaaacagttgtttgagaaagaagaaaaatgtgattagtattttaccttagcaatagtggcatagataatgataaattatagtcacacagaactcttagtatttacagaacgttcacatttgtgatcccatttaacaataactctgaaagaaaggtatcatctaccactgctttattgataaagatataaaaggtaagagagatgaaacatattggccaatgatacccatctggtaagagacagggatggggtgggaccccaaggctcttctcgccaagcccacggtttttttgctttatacttttttgcctcctgatcaccatggctgcagtttctactgtggacaatgtctgtcagcaagcattgatcccctgccttcagcactcttacgtcttagcaaggactggaaagaaaaagccaggagtttacagtctgctggagcaacagaaaagaatgatatgaaatatgaagagaccaaaatgatttataataaggtgctagactatgtagtaaaaatctgctttagctgtaagtcaaaagcaagagcagtcttttcagaatggaatagaaatgttggaattaaaggaattttcaaagttgtgaatttttttcaagataaacatgttttattttggtaattatggtattactaatttgataaccttcagggagccacctaatattatagaagatgtacatataatgacaaaagcaaacattttatttttaaggaccacaatctaatctaaaacaaaatttcccccttttctggtctttggttaattaaggacttatttaaatatcaaagaaagacacatagaaaacatttagtatatttctatacttttattaatgtcctccataccttacacagatacttgacttggctatggtctagataatccatgaaaatttaaaggacagattttaacaactttatgctaaattgatagatctctaggatcagattgccatcactctcagatgcgaagcttccaaccacttataggttcctgatatcttgcttttatacagacctaatttctcttcctttaaactttcttttcctcagttgctatttgattgaaatattgagtcattaaaaatttccaagtgggaatttttgtgtttcttcatctatcatgaagctgctcaaataagtaggtgtttgaataggagtagaaacagtaataggctgaagccagaccaatacagcttcagctaaatgccgaccttgctaaagtctgggaggaccggtgtggtattctacaatgtacaagtctgtagccggtgcccttaatatgttggcttcatgtctcatgactctcttctgtaaatatgcagtttaaaaaatacaagttattctgctgtagaagatacatttgcaaaattgatgtatcccctctaagtaaagttggctaaacaataaggacatatttataattaatgaatttgagaagaatgctgacgatatgcattattctttgaagttaacatttttcaggtcctaaataaacaaaaagtaggttacttctgtctggagtgtatgcaaggggtaccatcttgtccttggttcctggctgctattccaaggtgctataaagtcagctaaagagagcaatcataatacattgatagcatccctcaatgtgtttctgagctacttgagaatcttatttttgaataggtagcaggaaaccatctttgcagggcagcatgggcaaagggattggagggactattattataaagatccactgaactgcttcagtatcataatatcttaaactaaaggactggaaagagccagattccaatttaatctgctcttctatgaattcttagctgggttcatttaaaaagaaaaaacttgaagattgcaagattttgaagacatcttaaaataggtgaactccaaggtgcactttaaacttgagactgataactgaatactccttcaccttttgatctgatattgtcaaaatgaatgaggacttagtgctctagtaagtttggaacagaatgatattaatttattttctcatgattgattcttttttgctttttaatagattaaacttcaccgtagaacagtttctcaacctctggactattgacatttttgattggataattctttgctgtcagggctgttctgtgtgttgcaggatagttagcaacatccctgacaatcacaaatgttactttctgtctctatggatttgcctattctggacatttcgtataaatagaatcatatatatgtggcttcttgtacctggcttatttcacttaacatgttttcaaggttcatccatattgtagcatgtaacagcacttcattttctttttatggctgagtaatattctgttatgtggatatactaccatattttgtctatccactccttagctgatggtcttttaggttgtgtccattctttggctattataaataatgctgttaagaacattcatatacaagtttctgtgtagacatatatctttatttctcttgtgtggatacctaggagtagaattactggatcatatgataactctatgtgttaccttttgaggaactgccaaacatttttctacagtggctgtatcattttacactcccatcagcaatgtataagaattccaatttctctgtccttgcctatatttattaactgtcttttcttattagccaactgctgtggttcgaatgtttgtcccctccaaaactcatgttggaacataatccccaatgtggcagtattgagatgtgaggcctttaagaagtgcttgggtcatcagaggtctgccctcatgaataggctaatccattcatgagttaatgtactaatgggttatcactggattgggactagtggctttataagaagaggaagagaactaatctagtaagctcagccttctcactatgtgattgctgccctgtgtcaccttgggactctgcagagagtcctccagcagcaagaagttcttcatcagctgtggccccttgaccttggacttcccagcctccagaaatgtaagaaatccatttcttttttttaataaattacacagtctcacgtattcagttataccaacagaacacagactaagacaccatcctattgggtatgggtatctcattgtgttttttatttgtgtctcccaaatgactaacgatgttgaacatcttttcatctgctttttggacatttgtgtattttctttgaagaaatgtctttaacattctttgcccattttaaaattaggttgtctttttattgttgagttgtcggtgtgtgtgtgtgtgtgtgtgtgtgtgtgtgtatctagaatatatgtgtatgtatatatgcagatatattctaaacactagacccttatgaaatatataatttgaggacaatttctcccatttaaaaggccatcttttcacttcttgatagtgtcatttgactcacaagtttttaatttttatgaagtccaatttattttttaattctttgtttttggcactgtatctttaaaaagttgcctgatctaaggtcaaactgattttcacctatgttttcatctaagaattatagttttagctcttacatttaggcctttgatccattttgaattaatttgtgtatatggtgtgaagtagggctctaacttattcttttgtgtaatgatacctagttgtcccagcaccatttgttgaaaagattattctttccccattgaatggtcttgataccttgttgaaatcaactgaccataaatatataggcttattcctggactcacaattctatgagtctgtatgtctaatcttatgccagtaccacactgttttgattattacatctttgtacaaagttttgaaattgggaaatgtgagtcttccaactttgttcttttttaagattactttgcctatattccgtgttcgttgcaaactcatatgaattttaaatcaactctccatttctggaagaaaaaaagaggcaattgaagttcagatagggattgcattgaacctgtagatcagtttggggaatattgccatcataacaattagtaggtcttccaacccatgaatacaagacttctttccatttctgtagatatttagtttctttcattaatattttgtagttttcaatataaaagtcttgtacttcgattaaatttattcttgaatattttgggttttgatgcttttatgaatttgttttcttaatttcactttaagattgttcattgctactgattagtaatgcaactgatttttgtgtgttgatttttgtatcctgcaacctagctgaaatcattgattagcataatagagtatttaatagatttaggatttctatatataagatcatgtcatctgcaattagagataattttacttcttccctttcaatctggacattttttacttctttttcttgcctagttgccctagctagaacctccagtgcagtgttgaatagcagtggtgagaatgagcatctttgtgttggtcttcatcttgtggggaaacctttcagtttaagtgtgttgttgtggggttttcatagttgtcctttatcagattgagaatgttcctttctgttcctagtttgttgagtgttttctttttgattgttttaatcaggaaagggcattagattttgtcaaatgctttttctgcagctattgagatttttgtgtgtttttctggtcttttatggtttatcacattaattgattttcatatgtcaaacaaaccctgtgttcttgggtttcatctcacttggttatggtttataatcctttttatatacttgtagattcagtttgccagtattttgttgaggatgcttgcatttatatttataagggatattggtctgttgtagctgaccagtaagtatagtaagctgtatagtttactaagtgttccctctgttttgggggagactttgagaagaaggattgttggtaattgttctttaaacatttggtaaaattcactagtgaagccatctggggtcttctttggaagttttttgattactaacttaatgtctttacttgtttgttataagtccattcagatttttttctccttgagtcatttttgacagttggttgaggaatttgttcatttcatgtagttatctaattggttagtgtataattattcatagtattcctttataatcttatttttttgctgtaaggtcagtcataatgttcactctttcatttcggattctggtaatttaagagtcttctctccttttttttcttggtcagtctagctaaagtaaagttttgtccgttttcaggggaacagctttttttttttttttgaggcagaatttccatcttgtcacccagtctagagtgcagtggtgcaatctcggctcattgcagcctccgcttcccgggttcaagagattctcctgcctcagcttgccaagtagctgggattacaagcgcccaccaccacgcctggctaattttttatatttttagtagagacggggtttcaccatgttgggcaggctggtctcgaactcctgacctcaggtgatctgcctgccttggcctcccaaagtgctgggattacaggtgtgagctaccgtgcccaacccagctttggttatttttgttgacctactctattgtttttctcttctctatttcacttatttctacactggtctttattattttcttccttatgcttgctttggacttagttcttctttttctagtctcttaaggtggataattaagttcctgatttgaattcttacttctttgtaaggtggtcatgtactgctatgaatttccttctcagaaatgtatatgctttcactgcatcccttaagatttggtatgttgtatttttgttttcatttgtctcaaggtatagtcttctgatttccattgtgatttcttccccctctaacccgtttattatttaggaacttgttgatttccacatacctgtgaactttccagatttccttctttgttaattctcagtgtcattccattctggtccgagaacatactttgtatgatttctatcttttaaaatttatttggcttgtcttatgacctaatacattgtctatcctggaggatgtttcatgtacacttgagaagaatgtgtattctgcttttgttgggtagagtgtttgacaggtgtgttggtacatagttctgttcaaatctgtttccttgcagatttctatctagttgttctgtctattggaagtaggatattgaaatctccaactaatattgctgaattgtttattgttttcttcagttctgtcactttttgctttatatattttgaaattctattgttaggtacaagtaagtttatgattattatatcttcttgatagattgattcttttatcattatacagtgccctataagaacaatttttatcttaagtctattggtctatattagtatagccacttcagctttcttttgtttactgtttgcatggaatattttcttcttttactttctatttgtgttcttgagtctaaggtgaatctctgtagatagcaattggatctgccaatctttgctttttatttggggagtttaaaccattgacatttaatgtaattattgatgaggaagattacttctgatattttgccatttgtttcctttattttgtgtctcttgttcttaaattcttccattactaccttctttcttttgtattacatattttctagtgtaacgattttaatttctttgtcatttcttttgttgtatgtttttagttattttcttagtggttgccacggagattttattgtcattttaacagcctaggttgggcacagtggctcatgcctgtaatcccagcactttgggagactgaggcaggaggatagcttgagtccaggagttcaagaccagcctgggcaacttactgagatactgtctctacaaaaaaatacaaaaattagccaggcatggtggtgtgtgcctgtagtcccagatgcttgagaggctgagttgggaggatagcttgagcccaggaggttgaggctgcagtgaactttgatcacaccactgcactccagcctgggtaccagggcaaaactagcccaaagaaatgaaggaaaaaaaaaatctaatttagattaatatcaactcaacttcaacagtgtataaaaactttgcctctgtatacctcttctgcttccactctgtgctgttattgtcatagattttcatctttctacactgtgtgtttatcaatgtagatttaaaaatattgcttagtagttgtctttagaatccgatacggagaaaaggagatataaacaaaagatgcatttttactgtcttgtatgtttacttatgtaattccctttcctgatgttgtatttctaaaggcaaagtagggttattgtgagtgtccttttgtttcaacctgaaagactccttttagcatgtgttggagatatgctaatgatggactctcacagtttttgttatctgggaatgtgttaatttatccttcatttttgaaggatagtgttggcaggatacagaattcttggttgacatgtaattctttcagcattatgaatatgtcatcgtactgtcttctgacctccatggtttctgataaggaatcaactgttaatcttattgaggatcacttgtttgtaatgacttgcttgtcgtgctgctttcaagattcattctttgcctttagcttttggtagtttgattgtgatgcatttaggtgtgtactttattagtctgttctacttggagtttgttgagctttgtagatgtatttcatcagatgtgtcaagttcttttgccactattttttttttaaataatctttttgcccctttccgctccttctgtcactctgattatttgtgtgttgctttgtttggtggtgtcccagaagtctctgagactctgtccagttttttcctccccattcttttttctttcacttcctcagactggatgatctcaatttgacctatcttcgagttcatggattttctcttctccaagtgacatctgtgagatgaatttttttctagagaatttttcatttcagttattctacttcaaaatttctctttggttcagttttatcattgctatctttatattattctcagtttaatgagatactgttttatactttcctttagttctttagacatagtttatgtcactgaatatatttaaaatagctgattttaagtcttttttttttatttttttggagatggagtctcgctctgtcacccaggctggagtgcagtggcacgatctcagctcactgcaagctccacctcctgggttcacgcaatgattttaagtctttgtctatgaagtctagtatctgggcttcctcaggcatagtttctgttttctttctttcttttcctgtgtacttcgtttctttgtataccttgtaattgttgttgttaactggacattttgaatattatagtgtaacaactctggcagtcagactgtctcccctccccagtatttgttgttggtgagtattgtagatgtttgtttagtgacttttcatggctaattctgtaaattttatattctttgaagattgtgggcaccctgaagtctctgtttgttagtttagtggtcacctaataattaacagagatttcattaaatgcctagaagcaaaatatcttccagtctttgcccatggcctctgtgtatgcattagggcaggccttgaactcttacccagggagtttacaaccctgccttagcctttactaccagcttctgcagagcattaaggtcaacaggtggtgagagtttggagcctactccatctttcctgagcgtatacacagccctactcatgcatgtggccctctagatttccaggaatatgttggaccctttcaaagcccttacagactccccagcttttcctctcaatctttagactagtgtgttgttttcttcaacagttatctgtcaggcagcagcaaattaagagattagcataaatgttttcaactcctccacccgtcatgtgccccagggaagcactaagccagttctaagttaggcaaaataaagacaatccttttgaggtggtcttccatggagtcaccagacaggtaaaccaaataattaattacaagtctttggctggatacagtggctcacacctgtaatcccggcactttgggaggctgaggcaggtggatcacaaggtcaggagattgagaccatcctggctaacacggtgaaaccctgtctctactaaaaaatacgaaaaaataggtggctgtggtggcgggcgcctgtagtcccagctactcgggaggctgaggcaggagaatggaatgaacccaggaggtggagcttgccgtgagccgagatcacactactgcactccagcctgggtgacagagcaagactccgtctcaacaaaaaaaaaaaaaaacaagtcttcatgaaagaggtccattctgctgtctttcataccaggaatgtggaatgtggactgttattttcatggctactgctaagctaggaatcaagggatagatggggactgggtaaaacaccacagagtttgctgttcttaccaagaataagctggggaagagggttgtttttgtttttcagtaaaaattccctgggctgcttcaagccgttgattaattttcaggttccgaaaaagttcagtttgacagtttttgccctttttatttgcttttatggatatgtagaacttgagttcttttttccaccagttttgctgacattgttttaaaagcactttttgtaaaacccaaatgttgtctctctcaaggctagccaataattaaaaatactgttactcccctttgattttggaaatgaattcgtattgaccaaaattcaatactagaggtctttcaagctgttttaccatttatctaaactttagaatctaatgattcctgtacattgtctagcatactggtggtcctcaattgtcataagttcaactttggaacaaatgaactttttgtgtgcaagtttccaattgtttggaaattacattgatgccccctccatcaaactgttattcgtgggacatctaggaatttcttacagcagctgacaaatatttcaagtcagtgcctggtagtactgtccaccaggcaacagcttcagtagtagagcgatctttatctataaggcagtgtttgagcaattgtttattagtgttttcctaactactcagaagaactatcaggggttatagaggtagctcagagagttgggtgcaagtagagaaatccacccggcttgcattacacatcttatttctagagaagctttcctttgaagaaagagttctaaggtttaaaaaattaccttgaatgccacttatattgcattttaattttattttagagaaatcaatggaaagtagaaaaattaaggcactgatactagtgttaagaatgttggttaaagcttctggcaattaattttttatttccttttttaattttattaaaatttaacaattttcagtttatgctgtaatccagaccaaggtttcaatctaatgaagttaatgccagtgttgctgctacctattttgtctttagtcattcagccatgcttcctacttatactgaataagctagcttaatctaacaatcaaaaaagaaagctgttgcctaagttaagaaaaacagtttgaactgttttcaaactaaatacccagtagactctctagttgttgacaggagaatgcttaattcagaattgtcctgcagtagatcattttatctcattcctgttcttctataggatagcttatttgtttgaaattgtatttaatatgttgtgatttttgtgtgcttgtttctatttttcactggatagactcaagataaaacctggtaccctgcagtgtagctatcagtttatagcagaggaaatttacattagaacttggctgtgtatttacatgtatctaacttggaggtcactctgcttactgttgatatatcagtcatattagatgagtccctaatgagataccagaaaccccggaaacatcattaggtggaacagtgtccttaatgctttattaagtgttataggtaagacaaagcctagtactatttgtggcatcaaggttaggtgtttaaagacctgtattcttctattgtcatgttgaaattgttcccttgatgtagcaatagaaaattttagattaggcttaagttaatcagcaaacaaagataaaagtctgatactatcctaaatattttgtgtttctaaataatttaacagtgatccaattagctactcctgtagaaatgtaattgataaacttttcactctcttttaaattgccatcttgaattttacctgttttttaaagctgtctcaagtcctctctaaaaaaaggcagtcatttataaatttagaaaagcttgatagcacagaaagtcacagaaaaatgtaaacatagtttaaaactgaattgtatacaagccactagaagtacttttattaagtttacaaatattagtagagtggaactcatgcatttaatatgtttgaaacttttgatcaaatactgtgctatgaaaaacattttagataattattctttaatcatgtgtgtgtaaaatgtggctttttttgacaaccaagtagcttttctgtgtgccaaactgtgactttaaaattttaaagtactcaacagagtaaacaaaccacaaataccacttaaactgtacacatttgcacatgcatttcctataaatagtacatgggtttcaagtcttcacttttgaaattcagaaatgggttttttctccttccagtagaaataaaaacttgatttattttatttatttatttattttatttttgagacggagtctcgttctgtggcccaggctatggtgcaggagggtgatctcagctcactgcaacctctgcctcctgggttcaagtgattctcctgcctcagcctgccgagtagctgggattacaggtgcctgccaccatgcccagctaatttttgtatttttagtagagatggggtttctccatgttgggcaggctggtctcgaactcctggcctcaggtgatctgtctgtctcagccttccaaagtgctggggattacaggtgtgagccaccgcatccagctaaaaacttgatttttaaaaatccaaatcgaagacagaattgtgtattttagtacatttattagcagccttgacgctataccatatggctgtttatcatttaaacagcttgtaaaagcaaacacttcaggattcatgagtggcagaaggactgagtactttgggaaataagagagaacttttgttgaggatggttgaggaagagtccaagacaataataggcagaataagcaaaaatctagagactcattgtaggcactcaagtatgtatttgttagaatgaatggctgaacttggtatattgaggaacactgagaaagccatactgactggaagatagttcctacaagaaactggtgagacatatgttacagtctagattttggtgagccttgttaaagtttgggctttatttttatacggggagaaagtttcacaggggtttggaaatgaggcttggagctgttaatggggacacagtgaggttttagggtagtggctttcaaactgtttaaatccaaactttgatgataaccctgacataactattgtttataacttccatttcagttgtattggttttatcaaaacatcttcattgatcttactgattgcttcctatgcagattaatattataaatttgaatgtacaaaggaagctttagcagtaaaatagcaacttttatctgtcttacgtattggaggttctgcataagatttaattttttttttttttgaaatggagttttgctcttgttcacggggctggagtgcaatggtgtgatctcggctcaccacaacctctgcctcccgggtttaagtgattctcctggctcagcctcccaagtagctgggattacaggcatgtgccaccatgcccggctaattttgaattttagtagagacggggtttctccatgttggtcaggctggtctcgaactcctgacctcaggtgatccgcctgcctcagcctcccaaagtgctgggattacaggcgtgagccaccgcgcccggccaagatttaattttttaaaagaaaatattttgctaagggtttggaaactcttgttttagcaagaatggattaagactgattaaaactaaaggcaaagaggaggctcttatgtttggaattctttgctaatatttacacaatataattctctccacaaatatttaatggtaccagatattagatggttataatggcaaaagtgttcaaaggatgctatcatattcatgattcatgatcaaaatgaacattataaggctatccctcttcagaattaaatacgttactcctgtggaaaacttgcttttaatgtagaagttgtcccagagcctttcttcctttctcatgtcctcttatgtccactgctgagctaacatgggtctcactgaatgattaagaaaaaacatcttaggtggggagttctgtatatagtaaatgtttaatttattggggtggtgaacgggaagtgctgctggcaagagaggatgggaagagaaatctacccaaatccttacccgctttacagaacataaacttcctattcagtagtacacaataacttaacgatcaaggcatcttaacttttctgttttcagatgaaagaactatcgtttggcttgatcaagtatttagtatttattcgttcactcaagtgcttacgtttttttgttatctcagggttttacgttagttattaaccaaaagaactagttttagttctggaagtctaaaatatataagagaaggtgaggagtaataagagaagatgaagggagactttcggaatggcctatgaacttctagtaactataccaccttaaaatagacaaattacaatgcagttatgaagatatgtatttttcagtgaagacaactaaaatgtttgcacagaattttcttttttattgagtgttagaaattctattttggagatactaccttgcacaacataaaaagaaaaagtgagtgtggaatctaggaatctacgtggctctaggaaattttttaagtgtggaaactgaaggagagcaagagaaagggagcatggcattcccctgtttgtagttcatgaggtgggtttaaattgccttttgccaatgcagctgcacactgaggattacagaattctttttaaatgtttgtagaattatttttcacttattaggtaaaacgtgtattttttgattttctccaatttcagctttctcatgttgctatgctcaattttgtataccatatatagttttgttaaattgacaaagtggtgttttttgttcttctttttcccattggttaaaatttaaagagaaagtggaagctagaaatttatctaaaaaatgtaactttccctgtaattattaaagtatcaaatctaaatttgaattttctttgtgcataatcttttttcaagctatttaccatgttgacaaacttgctttcctgtggcaaatacactagcaatacgttataaatatgtaactttcaacctatttacagttgatgcttttttagccctttggatttaaaatacaagcactgaagaggtgaggaagtaccactgctgcctcagcattatttcgaaattctgtttataaactatacaatttccaaggtcatgaatccagcacctttccaggtactaactattgggacaaagatagaatttgattttatttatttacctattgactgaagtctaacttaaatcttgcacctagtaagatcttagaaataacgtgtgtactctgacctgtaaactaatcctagtattctgtgtgtatattctttctcatttgggctcttaaaaggaaaagtaacgtacatctgatgatcattagcactgagctttttcagcaaaaagtatatgtttataaagaagtataggataatttagtaatttaataatgtgacaacatttgcgtgtgtttttttttttgagaaatacaaattgtgagaaacagaaaagtaaaagaagcagcagcagaaatatcactataggatcaaaagattgcaggaaccaaaactccaaaattattgggcataatgtactaaaaacagggcagtggaggaaagggacagtccagactagctctgagggtccaaagaaagtattaaatattgttactggagtgatttgctctgctatttgggcttgggaattaagtgaaattgttgatatactagacagatacttcccacccatttttctcttgataatcagggttcattttttctattttctatttctctggatgctccatttcttaatattaatattaatattaagctctcagtctttatgctaaaaattggttatttaaaacaatttaaatcaacttcagtctaattggcttaagttcaaatccattttaagatcgatattgtgtcctttaaaaattttatttaaaagatatttaaactgatgagaggatactacccattccactgataaactattactgtaagtttgtctattgagggctagttatttggtttaaaaatgctgagattatggaaagtggattggaatattttggagcaatattaaaaacagtatctgtaacaatttaataaacttataaattcctctttctctgttgatctatcttgaaaagacactctatgtctctaggcattccttctctgtggtgtgattggtagacagggagtaaacaacttactgtaaatgggcaccatgccagttggcttcaggcagcatcaagcttgtgactcacagtcagggttaggaaaatgccttttaacttgtttgtctctgcctcttttaaacattaaaggcacaactgtactaattattaagtatttcataaggtcttttagggcttataagatcttttaggaatggcctggaagttattagtactgtttcattgaatctgaatacctttaacatgataatgagaagtttttaaagggtggttttatagttaaacggaatttctcaaattggcttgctccttatgttgatttatttaggatcacatttgggagtttctctgccctactttcaatgtatttaatttactgaccatcactatttggggggaaaatgttatatgatatttagaaaccaagagttttggagtttttcccccattagatgtatttatttatttatttattattttttaaagacagggtcttgctctgtcacccaggctggagcacagtggcatgatcctagctcactgtattcttgaactcctgggctcagactgtcctcccacctcagcccaagtggctaagtatcaagtaagaatcacctggcaaattccaaggctgtataccagatttcctaaattagaattttggggttgggtatctgaattttagtaaagccctccaaatgtttctggtattgcttctaagaacaattgataacataatagctgtggccattataggggtattctgtcatatttagatataggcataccttgttttattgtacttcccaaatattgcgtgtttattttgttttgtttcacttacaaattgaaggtttgtggcaaccctatattaagcgagtctgtcagtgccatttttccaacagcttgtgctcattttgtgtctctgtgtcacattttggtaattctctcaatatatcaaactttttcatcatttttgtatctgttacgaccagtgatcagtgatctttgattttttctttttttttttttttttgagacggacttttgctctgtcacccaggctggagtgcagtggttcaatcttggctcacagcaacctctgcctcccaggttcaagcaatcctcctgcctcagcctccccagtagccgggcctacaggcgtgtgccaccacgcctggctaatttttgtatttttagtagagatggggattccccatgttggccaggctggtctcgaactcctgacctcaggtgatccgctcaccttggcctcccaaagtgctgggattaccgtgccagcctgatgttactattttaattgttttcaggcaccataaacctcacctgtataaggcaccgtacttaattggtaaatattgcgcatgatctgactgctcttccaactggccattccctgtctgtctccctcttcctgggactctcaaatccctgagagacaataatattaaaattaagctaattaataaccctacagtggcctctaagtgttgaagtgaaagagttgcatgtctctcactttaaataaaaagctagaagtggctaaacttagtgaggaaggcacatcaaaagccaagacaggccaaaagcaaggactcttgtactaaacagctaaattgtgaatgcaaaggaaaagctcttgaaggaaataactagtgctactccagcaaacatgtgaatgatcagaaagtgaaacagccttcttgctgatacgaagaaagttttagtggtctggacagaagatcaaaccattcacaacattcctttaagccaaagcttaactctcttcaattctatgaaggctgtgagaggtgagaaagctgcagaagaaaaattggaagctagcagaggtcggttgatgaggtttagggaaagaagccagcgctgtaacataaaagtgtaaggtgaagcagcaagtgctgatacagaaactgcagcaagttatgtagaagatctagctaagattactaaataatagattttccatgtagatgaaaaagccttttgttggaagaagatgccatctaggactttcatagctagaaaggagtcaatgtctggcttcagaggacaggctgacattcttgttaggggctaatgtagttggtgactttaagttgaagccaggtctcatttaccactccaaaaatccgaagacccttaagacttatgcttaatctactctgcttgtactctagaaatgaaacaacaaagcctggatgacagcacatctgtttatagtatgcttcactgaatattttaaggccactgtaaagacctgttcaactgctcagaaaaaaatgattactttcaaaatattgctgttcattgacagtgcacctgggctcacccaagagctctaatggaattgtacaacaagatggatgttgttctcatgcctgccaacacatcatccatttgtagcccatgaatcagggagtgatttcaagtttcaaatcagtacattttgtaaggctatagctgctatagacagtgattgctctggtggacctgggcaaagtaaatcaaaaaccttctgaaaaggattggccattctagatgctattaagaatttgtgattcgcaggaggaggtcaaaggatcaacattagtagcagtttgaaagaagttgattccaacagttatagatgaatttgaggggttcaacacttcagtttaggaagtcactgcagatgtggtagaaacagcaagagaactagaattagaagtggagcccgaaaatgtgacggaattgctgcaatctcatgagaaaacgtgaatggatgaggagttgcttcttatggacaaatgagcaaataaatttttttcttgagatggaatctactcctggtgaagattctgtgaaccttgttgaaataacaacaaaggatttagaatattacataaacttaattggtaaagcagcagcatggtttgagtggattcattccagttttgaaagagtttctactgtgggtaaaatgctatcaaacagcatctcgtgctacaaagaaatcttttatgaaaagaaaagtgaaacttcattgttgtctactttaagaaattgccacagccaccccaccttcagcaaccacctctctgatcagtcagcaggcatcaacactgaagcaagaccctccacaaggaaaaagattacaactcactgaaagttcaaatgattgttagcatttttaagcaatattttaagattaaggtaaatacatttttaaagacacaatgctattgcacacttaatagactacagtatagtataaatataacttttatatgtagtgggaaaccaaaaaattcgtctgacttgctttgttgcaatattcactttattgtggtctagaaccgaacctgaaatatctcagaggtatgcctgtattaatattattttgcaagtaaaaaacccagcatataaaaaaaacgtagaatatgttgagagttcagtaatatggatgaaaatgtttttctctaactgaagaacatgataaattataattagggaaggatataaaccaagaaaatatgtctgagatagccaattcttgcagttcataatatgaaaactcattataccaatctcagtaagaatacttttaatagctgttatttctttgggatatagaatttataaagtacacagtaatcttcttatgatcaatcctaggatcactttacaaccacttaccccatattacaatgtagtaccaagacaagcagaccaaattatagaaggacaaagtttttgctaagcatattttgtcatcagcataccgcattgtgtgtgcatgcatgtgtgtgtttgtgcatgtgtgtgattgtataaaatattagaaagccaccccagaaaagttaaatgactaggaatgttgtgaagggattaagctacccctaaaattatataacaaaactctcttcatctattattaggtcatctttagaacatcttctcttaaatttgttataggtctctctcatctgtttggattaaaattggtctgaaagcctaaaatggctttttacctatataattatttcccaactagcttgtagtataggtgcaaagctatcacacttgctaggttagtgaagtatgtaaaaactaccatctttcaattaggaaccatcggatagcttctacaggattgctggggagaacctttataaagaaagttatatctttataaattttttgtcattttacttagctgagaatataaaataagttagctaataatagagtagaaatgttttctgtaacagattaatattgatcaaatgtgttattaaatgctaaaacaccattttttttctctgtaagccatgtgtttcatgccacaacacaaaagggacaattgtctgtgttttatgacagttctgttctgtcagatgctgtttgttcattttggtgaataaatgaagagagccctggacacatctttttttcctcaacaaaagaggaaaattattcttgtctgtatgtctataatcctgactctttgaatggctttaatttttttaaagtcagcatttttttataaagataggtgtttggaatgtgggcgatatggctggacagttagattgggaccaaataatggaaggctttgaacatcatgctaagaggtttgggttttactctgaaggcagtagagaaccattatgtttttaagccaggattgacttgttctaagctgtaccttagaaatattactctggcagttgtacataggatgagctgtatgttgctttgttttgtttggggagacagttctcgaagagagactacatacgaaggcagttatatgagtcattactaaaggtctggcaagaagtagtaaaagcattaactggagtggtagcagtagggaaggaaataaaaggatagatgtgggagtcatttggaaagtatgaggcaattcattgaccttacagaatcactggttttctgcttccactccattcacattgacctttccaaggttatcagtgacctgcttgtccttaaattcagtgggcactttccagtaacctactgttggcaccagccctgtgctagacaccaggatcctgtttgtaaaggcatctgccagtggtttctgtgacacaattctgtttctagttttcctccttctacttctctagcctcttggcaagttcttctttcagagtttctcagagctttgtgctaggccctcttctcattttctccttctctaagtgatcccatccttttctgttgcttcagttaccatttgtccttatgcaaaggacagccatatctactgtatctccagctcagatgtatctctttgcctcctgacccatatttccaactatctaactgggtatcttttcttggatgagttataggtctctcaaacacaacatgtccagaataattcattgacttattctaaggcctgcttcctctttctcctgtagtccctatctcaggaaatatatggtgctatcaaccccaaagcagaaatctggacataatccctaactacccttttcccctctctgtgcacataatttcagtca

SEQ ID NO: 26 - DNA, 3791 bp, Artificial Sequence
  H. sapiens
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaaaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgcttttttc

SEQ ID NO: 27 - DNA, 3557 bp, Artificial Sequence
  H. sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaagccccgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggtagacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattg

SEQ ID NO: 28-29 - skipped (2 entries)

SEQ ID NO: 30 - DNA, 20 bp, Artificial Sequence
  Antisense Compound
ttgatgtaggtcattctaat

SEQ ID NO: 31 - DNA, 20 bp, Artificial Sequence
  Antisense Compound
tggcttagtaaatatgttaa

SEQ ID NO: 32 - DNA, 20 bp, Artificial Sequence
  Antisense Compound
cttcccttcccagattagtg

SEQ ID NO: 33 - DNA, 20 bp, Artificial Sequence
  Antisense Compound
aaccatcatccacagtttac

SEQ ID NO: 34 - DNA, 20 bp, Artificial Sequence
  Antisense Compound
agttggtaaggtgcacacag

SEQ ID NO: 35 - DNA, 20 bp, Artificial Sequence
  Antisense Compound
gaaacctggtattgcctttg

SEQ ID NO: 36 - DNA, 20 bp, Artificial Sequence
  Antisense Compound
accagacagtaatagctata

SEQ ID NO: 37 - DNA, 20 bp, Artificial Sequence
  Antisense Compound
tagcttgtgaacgcagaagg

SEQ ID NO: 38 - DNA, 20 bp, Artificial Sequence
  Antisense Compound
cttgcagtcctcattcgagt

SEQ ID NO: 39 - DNA, 20 bp, Artificial Sequence
  Antisense Compound
ttcactgcacacaggaccag

SEQ ID NO: 40 - DNA, 20 bp, Artificial Sequence
  Antisense Compound
acttagcttgtgaacgcaga

SEQ ID NO: 41 - DNA, 20 bp, Artificial Sequence
  Antisense Compound
tagaatccaagagttttgtc

SEQ ID NO: 42 - DNA, 20 bp, Artificial Sequence
  Antisense Compound
agattagtgaataccaatat

SEQ ID NO: 43 - DNA, 20 bp, Artificial Sequence
  Antisense Compound
tgccgccctcctaacatgtt

SEQ ID NO: 44 - DNA, 20 bp, Artificial Sequence
  Antisense Compound
tgattgagaagcgacagcca

SEQ ID NO: 45 - DNA, 20 bp, Artificial Sequence
  Antisense Compound
gaaaatttcatccagccaac

SEQ ID NO: 46 - DNA, 20 bp, Artificial Sequence
  Antisense Compound
gtgagaggaattactttgtc

SEQ ID NO: 47 - DNA, 20 bp, Artificial Sequence
  Antisense Compound
cgactcaactgcttctgttg

SEQ ID NO: 48 - DNA, 20 bp, Artificial Sequence
  Antisense Compound
ctataccagttaggactgtt

SEQ ID NO: 49 - DNA, 20 bp, Artificial Sequence
  Antisense Compound
aataattttcaacagtgaag

SEQ ID NO: 50 - DNA, 20 bp, Artificial Sequence
  Antisense Compound
taccaggattttcagaggtt

SEQ ID NO: 51 - DNA, 20 bp, Artificial Sequence
  Antisense Compound
gcacacagaaagggctacta

SEQ ID NO: 52 - DNA, 20 bp, Artificial Sequence
  Antisense Compound
tctccaccccagagcaaatg

SEQ ID NO: 53 - DNA, 20 bp, Artificial Sequence
  Antisense Compound
actattgttttgttaccagg

SEQ ID NO: 54 - DNA, 20 bp, Artificial Sequence
  Antisense Compound
agtcattctctgctcattaa

SEQ ID NO: 55 - DNA, 20 bp, Artificial Sequence
  Antisense Compound
tccaaaaatgtttggaagca

SEQ ID NO: 56 - DNA, 20 bp, Artificial Sequence
  Antisense Compound
tggcccttcaaatgttgctg

SEQ ID NO: 57 - DNA, 20 bp, Artificial Sequence
  Antisense Compound
agaaaatttcatccagccaa

SEQ ID NO: 58 - DNA, 20 bp, Artificial Sequence
  Antisense Compound
tcagctgtgttacagctggt

SEQ ID NO: 59 - DNA, 20 bp, Artificial Sequence
  Antisense Compound
tggacagatctggctgctgc

SEQ ID NO: 60 - DNA, 20 bp, Artificial Sequence
  Antisense Compound
attctccactgaagcagata

SEQ ID NO: 61 - DNA, 20 bp, Artificial Sequence
  Antisense Compound
cccctagagcaaactgtttg

SEQ ID NO: 62 - DNA, 20 bp, Artificial Sequence
  Antisense Compound
attgctggtacctctatgca

SEQ ID NO: 63 - DNA, 20 bp, Artificial Sequence
  Antisense Compound
tcagtgaatatcaactctgg

SEQ ID NO: 64 - DNA, 20 bp, Artificial Sequence
  Antisense Compound
cacatattaaggtttctaat

SEQ ID NO: 65 - DNA, 20 bp, Artificial Sequence
  Antisense Compound
atatataacatgtcatgata

SEQ ID NO: 66 - DNA, 20 bp, Artificial Sequence
  Antisense Compound
aacacttcaggttcaataac

SEQ ID NO: 67 - DNA, 20 bp, Artificial Sequence
  Antisense Compound
gggtgaagacgcagaaacct

SEQ ID NO: 68 - DNA, 20 bp, Artificial Sequence
  Antisense Compound
tctcccatatacagtcccat

SEQ ID NO: 69 - DNA, 20 bp, Artificial Sequence
  Antisense Compound
gtttgcaatgctttcttcca

SEQ ID NO: 70 - DNA, 20 bp, Artificial Sequence
  Antisense Compound
acctattgaggtttgcaatg

SEQ ID NO: 71 - DNA, 20 bp, Artificial Sequence
  Antisense Compound
ctggtcgacctattgaggtt

SEQ ID NO: 72 - DNA, 20 bp, Artificial Sequence
  Antisense Compound
ctgtggtatacaatttcaca

SEQ ID NO: 73 - DNA, 20 bp, Artificial Sequence
  Antisense Compound
ctttggtctgtggtatacaa

SEQ ID NO: 74 - DNA, 20 bp, Artificial Sequence
  Antisense Compound
caaaggtgctttggtctgtg

SEQ ID NO: 75 - DNA, 20 bp, Artificial Sequence
  Antisense Compound
gaaaactccaaatcctgcaa

SEQ ID NO: 76 - DNA, 20 bp, Artificial Sequence
  Antisense Compound
ggtttagtgtccggtaaaat

SEQ ID NO: 77 - DNA, 20 bp, Artificial Sequence
  Antisense Compound
ttctcttgcttaattacccc

SEQ ID NO: 78 - DNA, 20 bp, Artificial Sequence
  Antisense Compound
gcccagtttctcttgcttaa

SEQ ID NO: 79 - DNA, 20 bp, Artificial Sequence
  Antisense Compound
gaaatggcagacattttatt

SEQ ID NO: 80 - DNA, 20 bp, Artificial Sequence
  Antisense Compound
ccatgaacagaaatggcaga

SEQ ID NO: 81 - DNA, 20 bp, Artificial Sequence
  Antisense Compound
tgtcctccagaggtactcac

SEQ ID NO: 82 - DNA, 20 bp, Artificial Sequence
  Antisense Compound
gtggtacatctgtcctccag

SEQ ID NO: 83 - DNA, 20 bp, Artificial Sequence
  Antisense Compound
tcatgtcatagtggtacatc

SEQ ID NO: 84 - DNA, 20 bp, Artificial Sequence
  Antisense Compound
gatgctgtattcatgtcata

SEQ ID NO: 85 - DNA, 20 bp, Artificial Sequence
  Antisense Compound
tgagaaagggatgctgtatt

SEQ ID NO: 86 - DNA, 20 bp, Artificial Sequence
  Antisense Compound
tggtggaatgacattaaaaa

SEQ ID NO: 87 - DNA, 20 bp, Artificial Sequence
  Antisense Compound
ggaattggtggaatgacatt

SEQ ID NO: 88 - DNA, 20 bp, Artificial Sequence
  Antisense Compound
gagcacaccaggcagagttt

SEQ ID NO: 89 - DNA, 20 bp, Artificial Sequence
  Antisense Compound
ctgtccttccactgctcttt

SEQ ID NO: 90 - DNA, 20 bp, Artificial Sequence
  Antisense Compound
ggtaattgtgctgtccttcc

SEQ ID NO: 91 - DNA, 20 bp, Artificial Sequence
  Antisense Compound
tttcgatagcggcatgctgg

SEQ ID NO: 92 - DNA, 20 bp, Artificial Sequence
  Antisense Compound
tgaagacattttcgatagcg

SEQ ID NO: 93 - DNA, 20 bp, Artificial Sequence
  Antisense Compound
gtttttcgagcttccaggtt

SEQ ID NO: 94 - DNA, 20 bp, Artificial Sequence
  Antisense Compound
caggaactattgttttgtta

SEQ ID NO: 95 - DNA, 20 bp, Artificial Sequence
  Antisense Compound
gcctttgcccatttcactgc

SEQ ID NO: 96 - DNA, 20 bp, Artificial Sequence
  Antisense Compound
taataatcagatcaggagca

SEQ ID NO: 97 - DNA, 20 bp, Artificial Sequence
  Antisense Compound
tgctcattaataatcagatc

SEQ ID NO: 98 - DNA, 20 bp, Artificial Sequence
  Antisense Compound
cattctctgctcattaataa

SEQ ID NO: 99 - DNA, 20 bp, Artificial Sequence
  Antisense Compound
cagggtagagtcattctctg

SEQ ID NO: 100 - DNA, 20 bp, Artificial Sequence
  Antisense Compound
agcatgtgtttacattggtc

SEQ ID NO: 101 - DNA, 20 bp, Artificial Sequence
  Antisense Compound
atacagagatactcttcata

SEQ ID NO: 102 - DNA, 20 bp, Artificial Sequence
  Antisense Compound
taaggttttcatacagagat

SEQ ID NO: 103 - DNA, 20 bp, Artificial Sequence
  Antisense Compound
gagagaagcagtaaggtttt

SEQ ID NO: 104 - DNA, 20 bp, Artificial Sequence
  Antisense Compound
ggcttttcctagctctttga

SEQ ID NO: 105 - DNA, 20 bp, Artificial Sequence
  Antisense Compound
ttgacaatggcttttcctag

SEQ ID NO: 106 - DNA, 20 bp, Artificial Sequence
  Antisense Compound
gtgatgatttcagctaacat

SEQ ID NO: 107 - DNA, 20 bp, Artificial Sequence
  Antisense Compound
tgccaagtcttggccctcta

SEQ ID NO: 108 - DNA, 20 bp, Artificial Sequence
  Antisense Compound
ctgcttctgttgccaagtct

SEQ ID NO: 109 - DNA, 20 bp, Artificial Sequence
  Antisense Compound
caggagggaaatatattttt

SEQ ID NO: 110 - DNA, 20 bp, Artificial Sequence
  Antisense Compound
acaacttagcttgtgaacgc

SEQ ID NO: 111 - DNA, 20 bp, Artificial Sequence
  Antisense Compound
ctctggcagaggagccgctc

SEQ ID NO: 112 - DNA, 20 bp, Artificial Sequence
  Antisense Compound
tccatcagtgaatatcaact

SEQ ID NO: 113 - DNA, 20 bp, Artificial Sequence
  Antisense Compound
tttggagtccatcagtgaat

SEQ ID NO: 114 - DNA, 20 bp, Artificial Sequence
  Antisense Compound
atgattctttggagtccatc

SEQ ID NO: 115 - DNA, 20 bp, Artificial Sequence
  Antisense Compound
ttatagaagtccatcacatc

SEQ ID NO: 116 - DNA, 20 bp, Artificial Sequence
  Antisense Compound
acgcagaaaccttcacagta

SEQ ID NO: 117 - DNA, 20 bp, Artificial Sequence
  Antisense Compound
actgctttggacagatctgg

SEQ ID NO: 118 - DNA, 20 bp, Artificial Sequence
  Antisense Compound
gtatacaatttcacattgcc

SEQ ID NO: 119 - DNA, 20 bp, Artificial Sequence
  Antisense Compound
caaaatgtcaaaggtgcttt

SEQ ID NO: 120 - DNA, 20 bp, Artificial Sequence
  Antisense Compound
aggtctgatctccaaggact

SEQ ID NO: 121 - DNA, 20 bp, Artificial Sequence
  Antisense Compound
tccaaaaggaatgaatcgtc

SEQ ID NO: 122 - DNA, 20 bp, Artificial Sequence
  Antisense Compound
ggcagacattttattaccaa

SEQ ID NO: 123 - DNA, 20 bp, Artificial Sequence
  Antisense Compound
tcctgctgttgagaaaggga

SEQ ID NO: 124 - DNA, 20 bp, Artificial Sequence
  Antisense Compound
cagatccttggcacctattc

SEQ ID NO: 125 - DNA, 20 bp, Artificial Sequence
  Antisense Compound
ccccagagaagtcaagttgt

SEQ ID NO: 126 - DNA, 20 bp, Artificial Sequence
  Antisense Compound
ctgttgttgctgttgaggag

SEQ ID NO: 127 - DNA, 20 bp, Artificial Sequence
  Antisense Compound
caggttcaataacctccaac

SEQ ID NO: 128 - DNA, 20 bp, Artificial Sequence
  Antisense Compound
cgccctcctaacatgttgag

SEQ ID NO: 129 - DNA, 20 bp, Artificial Sequence
  Antisense Compound
ttcctgaaacctggtattgc

SEQ ID NO: 130 - DNA, 20 bp, Artificial Sequence
  Antisense Compound
aacacagcaggtttgcactt

SEQ ID NO: 131 - DNA, 20 bp, Artificial Sequence
  Antisense Compound
tccttaggaactgaagagag

SEQ ID NO: 132 - DNA, 20 bp, Artificial Sequence
  Antisense Compound
catcaaatagctcttggctc

SEQ ID NO: 133 - DNA, 20 bp, Artificial Sequence
  Antisense Compound
ctctttgatgtaggtcattc

SEQ ID NO: 134 - DNA, 20 bp, Artificial Sequence
  Antisense Compound
atccaagagttttgtcagtt

SEQ ID NO: 135 - DNA, 20 bp, Artificial Sequence
  Antisense Compound
tttcaaccacttcatgcata

SEQ ID NO: 136 - DNA, 20 bp, Artificial Sequence
  Antisense Compound
gtatctgattggtgatgatt

SEQ ID NO: 137 - DNA, 20 bp, Artificial Sequence
  Antisense Compound
taaggcagtcacttttgatg

SEQ ID NO: 138 - DNA, 20 bp, Artificial Sequence
  Antisense Compound
taattcgactttctttaagg

SEQ ID NO: 139 - DNA, 20 bp, Artificial Sequence
  Antisense Compound
acaaactgatagtttataca

SEQ ID NO: 140 - DNA, 20 bp, Artificial Sequence
  Antisense Compound
gtgcgtatttaaaacaaaac

SEQ ID NO: 141 - DNA, 20 bp, Artificial Sequence
  Antisense Compound
taatttctccaaaatactga

SEQ ID NO: 142 - DNA, 20 bp, Artificial Sequence
  Antisense Compound
aaaagtgatgacgactcaac

SEQ ID NO: 143 - DNA, 20 bp, Artificial Sequence
  Antisense Compound
ttacgtccacatattaaggt

SEQ ID NO: 144 - DNA, 20 bp, Artificial Sequence
  Antisense Compound
ttaggtgccatccttctttg

SEQ ID NO: 145 - DNA, 20 bp, Artificial Sequence
  Antisense Compound
gcactggtggtttaggtgcc

SEQ ID NO: 146 - DNA, 20 bp, Artificial Sequence
  Antisense Compound
tttgggcactggtggtttag

SEQ ID NO: 147 - DNA, 20 bp, Artificial Sequence
  Antisense Compound
atttcatccagccaactgtg

SEQ ID NO: 148 - DNA, 20 bp, Artificial Sequence
  Antisense Compound
ggatacaccaacagaaagtc

SEQ ID NO: 149 - DNA, 20 bp, Artificial Sequence
  Antisense Compound
acaacttcccttttctgata

SEQ ID NO: 150 - DNA, 20 bp, Artificial Sequence
  Antisense Compound
cagtaatagctataaaaggc

SEQ ID NO: 151 - DNA, 20 bp, Artificial Sequence
  Antisense Compound
agcaagcgtagttcactaaa

SEQ ID NO: 152 - DNA, 20 bp, Artificial Sequence
  Antisense Compound
gctgcccatcttaaacagct

SEQ ID NO: 153 - DNA, 20 bp, Artificial Sequence
  Antisense Compound
aagcaccaacccattttcac

SEQ ID NO: 154 - DNA, 20 bp, Artificial Sequence
  Antisense Compound
ccatcaggttagaagcacca

SEQ ID NO: 155 - DNA, 20 bp, Artificial Sequence
  Antisense Compound
ttctgatagctaagtgccat

SEQ ID NO: 156 - DNA, 20 bp, Artificial Sequence
  Antisense Compound
aagaatactggagatttgag

SEQ ID NO: 157 - DNA, 20 bp, Artificial Sequence
  Antisense Compound
gctctataccagttaggact

SEQ ID NO: 158 - DNA, 20 bp, Artificial Sequence
  Antisense Compound
ttacccagcaggtcactgga

SEQ ID NO: 159 - DNA, 20 bp, Artificial Sequence
  Antisense Compound
catccacagtttacccagca

SEQ ID NO: 160 - DNA, 20 bp, Artificial Sequence
  Antisense Compound
tagtcttttgcaaccatcat

SEQ ID NO: 161 - DNA, 20 bp, Artificial Sequence
  Antisense Compound
agggcctcttggtagttatt

SEQ ID NO: 162 - DNA, 20 bp, Artificial Sequence
  Antisense Compound
tagccattgcaaaaataggg

SEQ ID NO: 163 - DNA, 20 bp, Artificial Sequence
  Antisense Compound
tgccatatagccattgcaaa

SEQ ID NO: 164 - DNA, 20 bp, Artificial Sequence
  Antisense Compound
ctgaaagacaaatagtttac

SEQ ID NO: 165 - DNA, 20 bp, Artificial Sequence
  Antisense Compound
acaacttttaagaagttata

SEQ ID NO: 166 - DNA, 20 bp, Artificial Sequence
  Antisense Compound
tggttatctggaatcacaac

SEQ ID NO: 167 - DNA, 20 bp, Artificial Sequence
  Antisense Compound
acagctggttatctggaatc

SEQ ID NO: 168 - DNA, 20 bp, Artificial Sequence
  Antisense Compound
agtctctcagctgtgttaca

SEQ ID NO: 169 - DNA, 20 bp, Artificial Sequence
  Antisense Compound
gtgaaaatgggtgtctagcc

SEQ ID NO: 170 - DNA, 20 bp, Artificial Sequence
  Antisense Compound
tgacagatgggaatgtgaaa

SEQ ID NO: 171 - DNA, 20 bp, Artificial Sequence
  Antisense Compound
aaagattaaccaattggtga

SEQ ID NO: 172 - DNA, 20 bp, Artificial Sequence
  Antisense Compound
tttcctgtaccatcaggaaa

SEQ ID NO: 173 - DNA, 20 bp, Artificial Sequence
  Antisense Compound
tctatggcacacattaggga

SEQ ID NO: 174 - DNA, 20 bp, Artificial Sequence
  Antisense Compound
ttgtgttaaactctatggca

SEQ ID NO: 175 - DNA, 20 bp, Artificial Sequence
  Antisense Compound
aagaaattcacaggacttgt

SEQ ID NO: 176 - DNA, 20 bp, Artificial Sequence
  Antisense Compound
gaaagttggtaaggtgcaca

SEQ ID NO: 177 - DNA, 20 bp, Artificial Sequence
  Antisense Compound
caaatttcttgtggcttagt

SEQ ID NO: 178 - DNA, 20 bp, Artificial Sequence
  Antisense Compound
ttgaatagaaatcaaatttc

SEQ ID NO: 179 - DNA, 20 bp, Artificial Sequence
  Antisense Compound
acacaaataatttggccacc

SEQ ID NO: 180 - DNA, 20 bp, Artificial Sequence
  Antisense Compound
ctattacacaaataatttgg

SEQ ID NO: 181 - DNA, 20 bp, Artificial Sequence
  Antisense Compound
agtagcccttcccttcccag

SEQ ID NO: 182 - DNA, 20 bp, Artificial Sequence
  Antisense Compound
aaagctgcagtagcccttcc

SEQ ID NO: 183 - DNA, 20 bp, Artificial Sequence
  Antisense Compound
tgcatgtaaagctgcagtag

SEQ ID NO: 184 - DNA, 20 bp, Artificial Sequence
  Antisense Compound
attttaataaattgcatgta

SEQ ID NO: 185 - DNA, 20 bp, Artificial Sequence
  Antisense Compound
caagctattttacaatcatt

SEQ ID NO: 186 - DNA, 20 bp, Artificial Sequence
  Antisense Compound
atccatcagcatttctttga

SEQ ID NO: 187 - DNA, 20 bp, Artificial Sequence
  Antisense Compound
tataaatcatataggttatc

SEQ ID NO: 188 - DNA, 20 bp, Artificial Sequence
  Antisense Compound
caaactgtttggtttctgag

SEQ ID NO: 189 - DNA, 20 bp, Artificial Sequence
  Antisense Compound
ctgggtcagagcctcagcaa

SEQ ID NO: 190 - DNA, 20 bp, Artificial Sequence
  Antisense Compound
taatctcactgggtcagagc

SEQ ID NO: 191 - DNA, 20 bp, Artificial Sequence
  Antisense Compound
aatgagaagggtggtcagaa

SEQ ID NO: 192 - DNA, 20 bp, Artificial Sequence
  Antisense Compound
ctcactgttggaatgagaag

SEQ ID NO: 193 - DNA, 20 bp, Artificial Sequence
  Antisense Compound
agtaaactaaacctgcgctg

SEQ ID NO: 194 - DNA, 20 bp, Artificial Sequence
  Antisense Compound
ctgtttacatactttacata

SEQ ID NO: 195 - DNA, 20 bp, Artificial Sequence
  Antisense Compound
agatggtgcctttaaggatg

SEQ ID NO: 196 - DNA, 20 bp, Artificial Sequence
  Antisense Compound
atgtgaaagtaacccgctat

SEQ ID NO: 197 - DNA, 20 bp, Artificial Sequence
  Antisense Compound
ttctgaagcttctgttgtca

SEQ ID NO: 198 - DNA, 20 bp, Artificial Sequence
  Antisense Compound
ctggtacctctatgcaaact

SEQ ID NO: 199 - DNA, 20 bp, Artificial Sequence
  Antisense Compound
gagattctgcactatttaca

SEQ ID NO: 200 - DNA, 20 bp, Artificial Sequence
  Antisense Compound
tagtgtattattggcaacct

SEQ ID NO: 201 - DNA, 20 bp, Artificial Sequence
  Antisense Compound
ttatttggaaataaactctt

SEQ ID NO: 202 - DNA, 20 bp, Artificial Sequence
  Antisense Compound
aaaacatgtcctcattttat

SEQ ID NO: 203 - DNA, 20 bp, Artificial Sequence
  Antisense Compound
gaagctctttttgaaactta

SEQ ID NO: 204 - DNA, 20 bp, Artificial Sequence
  Antisense Compound
tggttttaaccacataacat

SEQ ID NO: 205 - DNA, 20 bp, Artificial Sequence
  Antisense Compound
acataacattttcatgcata

SEQ ID NO: 206 - DNA, 20 bp, Artificial Sequence
  Antisense Compound
tttgttgtgagtaaccaact

SEQ ID NO: 207 - DNA, 20 bp, Artificial Sequence
  Antisense Compound
acactaaaaatacttttcag

SEQ ID NO: 208 - DNA, 20 bp, Artificial Sequence
  Antisense Compound
aactccacccaaagggttta

SEQ ID NO: 209 - DNA, 20 bp, Artificial Sequence
  Antisense Compound
ttcctgaaaacctggtcact

SEQ ID NO: 210 - DNA, 20 bp, Artificial Sequence
  Antisense Compound
attaatctgcataggaagca

SEQ ID NO: 211 - DNA, 20 bp, Artificial Sequence
  Antisense Compound
ttctaccaacctgaagagag

SEQ ID NO: 212 - DNA, 20 bp, Artificial Sequence
  Antisense Compound
agaagaactcgtgatattat

SEQ ID NO: 213 - DNA, 20 bp, Artificial Sequence
  Antisense Compound
tccttaggaactaaaaggtt

SEQ ID NO: 214 - DNA, 20 bp, Artificial Sequence
  Antisense Compound
tttcaaccacctgcaagaga

SEQ ID NO: 215 - DNA, 20 bp, Artificial Sequence
  Antisense Compound
ggtcccagctgcttcggccg

SEQ ID NO: 216 - DNA, 20 bp, Artificial Sequence
  Antisense Compound
ggagagcccctatttaagaa

SEQ ID NO: 217 - DNA, 4734 bp, Artificial Sequence
  M. musculus
gcactgggatgggttttccttcttgaggtgtcaagcttcggctcctttgccaagatggcggccctggctcccatggaggtagcgattgtgcagcacctctggcccaggggccgtcttacagccacggcctacccgcgcaaagcggaagacacattgggcagcctttacattttccatccaagaaagggcgcctcggttttgaagctaaagagcacctctgccaaaatggtgaccgtgtggcgtcactgctctttaccaagatggcggcgagagacttccggcacgcgcttccccaatcagagatctccaagaggtcaggcagaggagaccgccctcggagtccaagtgctgcgcgatcggctgggggagagaatggggtggagccacggcagcctcagctccgatcagaagtgccaagcgctggcacctgtgggggagcaaaagttacttcctcgcaccccaaagcaacaccataacaccttactccccaacccccaggcccctaaaccgtagcgtgcggcgcgaggtagggggcacggtcccagagtcgccccagtctgccaaaggggcgggcagcttaggggcaggtccgcgcggcggctgcactgtcacccccacgcccctctcctgtcctagggggaccggccacgtgtttctcttggagacccggggctcgccctgggaacagctggagggagctaaacgctgacgttgtaaagatgcgtgttttgttttatttggaggggcagaggggtccctggaactcagaaagaaggcagagcgaggcactgagcctggagcagcaaatgtcaagatttgggggaggggcctccgcggggagcttggatgctggccccgaagggggtggaaggagaggtcaggagtttggggtaagaggagggcggacttcgccagcaacttactattccgtctgcaacttgcttctaggcctgcacacaccccctcccggccccgcaaggcttccttaatcacaatttttttcttttttctttttttttcttttttttttttaagtgcaaagaaacccagctcgctaagagggttttgcattcgccgtgcaacttcctccgaatgtgagcgcgctggcaggcagggagggagcggtgggggggggggggggaggtttgaacttggcaggcggcgcctcctgctgccgccgccgccgctgctgccgcctcctctcagactcggggaagagggtgggggacgatcggggcgcgggggagggtgggttctgctttgcaacttctcccggtggcgagcgagcgcgcgcgcagcggcggcggcggcagcggcggcggctgcagacggggccgcccagacgatgcgggggtgggggacctggcagcacgcgagtccccccccgggctcacagtatgtatgcgctgaccctctgctctgccctcccgtcccctccccaagcctccccccaccccgagggcgtgtctgccgtcctgtcccgagagcggccagggctctgcggcaccgtttccgtgccatcccgtagcccctctgctagagtgacacacttcgcgcaactcggcagttggcggacgcggaccacccctgcggctctgccggctggctgtcaccccctcgggggctctggcggcagacccacggggcgggctcccgagcggttccaagccgcggagctgggcgggggcgggaaggagccagggagaagagaaactaaagaaactcgctttccctcccaggccaggtcggcagccgctgccgcactttttttttttctcgtccctcgggtggggaaaaaaggccaagaagccgcgcggcccccggcggtggcgatgcccggagccgcgcgggcttgcaggcgccgtcggggccgggctggcggggcccgcgcgctgggcgagaagagggcgagggccgagggcgcccttgcagttgccgacagtcgccaacaggttgcaccgttccccgcggccgccgcgcggcccctcgggcggggagcggccggggggtggagtgggagcgcgtgtgtgcgagtgtgtgcgcgccgtggcgccgcctccgcccgcccctcgctcggtcccgctcgcctgccgcggccgggcggccctttcgcgtgtccgcgctccccccccttcccctccgcctcctccattttgcgagctcggagtcagtgcctggagcccgagtcgccgcccgcccgtcggggacggattctaagtgggtggaacaagacgccgcagccgggcggcgcggcgccgggacgggagaacgcgcgcggggagacgggagcggcgcgggggcccggcttgtcagccgggaaggggtgactttccgcgctaggggctctcccctcccccatggaaaagagggggcgactgttgacttccttctccgtgacacgcgcgcctcccgcgtccgcacgccgacttgtttatctggctgcggtgggagcgcaggcgggcgggcgagcgcgcgggtgctgaggtgagcgggggctgggcgagcgggcgcccgcgctgaggtgagccggactgggcgcgctccccgaggggctcggcagccggggcggcgggacttggcaaacttttgccagcccgggcttgggggcgggggagggggggtggaggctggcgagggcagggtgacggtgacgaaagggcctcggcggtgacagcgctggcgcttcctctccccgcaccgccatccctggcccagcgcgctgccccgccgcggagcctcgagcgcctggcgggagtctggcgtccttttcggttttgcttttttttttttttttccttgtcgcaagccctccggtctccgctgtcctcggggccgccaggacagccagctttttccccctgggggaaggcagaggggcggctgcgacccgcggtctcagggcgcgcggctgcgggctgcgggcttgtggggtggattcggggctcgctgcctgcagcccagacttcgcccgcccggccttatctgctagaagtgggcgtgccgcagagaactcaacaggtctggacacattctcccttcacctcgcacctttctccctccttctcccccaaccccacccccggacaacttgggcactagctttggggcatgatttcgaacctgacttttctgagtggtccccttttagaaagagaccctccctagccacagttttttgatgcagcgattttttttttttttttttttttaacaagtgctactttgacgtttgaggttgcaggctcaggaattgcggccttaccacttaagaccctgggcatggtttcctgtgactaatgtcacagggttatttacagttttaaatgggggataaatgtcgcttaagggagcatcttgttttatgaagtgttgttatggtttcaggctggaaggggcagctgtcaaaaaagcgggtgtgaaaattcattaaggtcatttgataactaatgtttagagaccgttctcagaggcataggaccccggttctgagagacaagtcctgtacagggaagaagcctacggcaggtcccagccctctctccaccttcctcaaattccccttccagagaagaaggtcatacttagtgtcttggtacaggaaaaatttaccattttatgggaggggattccaaatatatttgtcgtagtctttctagcctctcaaaacattttgattattaataatatctagcagtctggaggaataaataaaggtttaaaactgcagagaaaattatctgagcatttcttgaatgggttttatttgactttatatgtgattctacagtccagtttttctgtttttcctggtatttacatatttggaaagagaaatcttcaacttatagataaaatattttgtattgagcatattaaacaaatttttaaaaaaggaatataactcagtaaatcttcgtgttaggaattttgataagctctgttctattaaactatttaaatacgttaaaattgtagggaagaagccagtacacactctactctgcttttaaggatgcacattttagtctgtatataatatatattatttcttatatcccaagtttgaaggtaggtgatactagacaggcatatttagttgaaaatggagtttcagagtaagagcttttccctagccctgcaagacaagcagcccagcactgctttttttttttttccaataaaatgtgcataattcttcaaggttgtaccttgaaaggtttagatgtgtggggattggcacaggtgaaattgtgaatcatgaggtgtgtaaacaattatagtatagcatttatcaaacggtttatgtagtggtttccagagaggaatctctcaattgaaaggggttggaaatttaaggatcacgcctttaaaaaaaaaaaaaaaaagttaaatactttgacgtaaatttgaacctttgcaataatcacaaataacaaattacaacctccatgcttaccaaatgtgtacatttagcgagtggcaggataaacagccaagtttaaatcggtcgaccgggcagatgagctgctttgtggatgggagcagggaggatatagctttattttagaagataggaactattcttctgataatgaagactttggtttgggagttacctaaagggtttatttaataggctgtctcttactaatcggatcagagataatgtggttttatagcttattatgtccttttttttttttttgtagttaatatttgccaatggactccaaa

SEQ ID NO: 218 - DNA, 836 bp, Artificial Sequence
  M. musculus
ggacgcgtggggcggcggcggcggcagcggcggcggctgcagacggggccgcccagacgatgcgggggtgggggacctggcagcacgcgagtccccccccgggctcacattaatatttgccaatggactccaaagaatccttagctccccctggtagagacgaagtccccagcagtttgcttggccgggggaggggaagcgtgatggactgtataaaaccctgaggggtggagctacagtcaaggtttctgcgtcttcaccctcagtggctgctgcttctcaggcagattccaagcagcagaggattctccttgatttttcaaaaggctcagcaagcaatgcacagcagcagcagcagcagcagctgccgcagccagatttatccaaagccgtttcactgtccatgggactgtatatgggagagaccgaaacaaaagtgatggggaatgacttgggctacccacagcagggccagcttggcctctcctctggggaaacagactttcggcttctggaagaaagcattgaaacctcaataggtcgaccagccgtccagagaatcccaagagttcaacacctgcagctgggtgtgctaccccgacagagaaggagtttccccagactcactctgatccatcttcagaacagcaaaatagaaaaagcagctggaccaaggtggcaggtgtgaaaattgtgtaccaagacaaagcacttttgacatctggagggatgggggttctggcgggtcccagggtaagagaaaagagagtcttggggggccacctgtggttggatgcaaactggttccttgggggggaaaaggtccccctgggggg

SEQ ID NO: 219 - DNA, 571 bp, Artificial Sequence
  M. musculus
gcccgtcggggacggattctaagtgggtggaacaagacgccgcagccgggcggcgcggcgccgggacgggagaacgcgcgcggggagacgggagcggcgcgggggcccggcttgtcagccgggaacgggtgactttccgcgctaggggctctcccctcccccatggaaaagagggggcgactgttgacttccttctccgtgacacgcgcgcctcccgcgtccgcacgccgacttgtttatctggctgcggtgggagccgcaggcgggcgggcgagcgcgccggtgctgagttaatatttgccaatggactccaaagaatccttagctccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcgcagcagcagcagcagcagcagcagcagcagcagcagcagcagcagc

SEQ ID NO: 220 - DNA, 19000 bp, Artificial Sequence
  M. musculus
aggattagagggataagtaacaactcccagctacagttactattttttaatttttgttcctaaagaagcaattgaaaaacattgttatttctaactagagaaagaacaatttttagttgtgaatgtattaatctttttttttttttttgcatttgaggcttagcgcttcgctaggctgaaataaaagcatgcataaaggatgaagagacaagcaacatccagtctagcttattcctaagtaccatctcttagcaaagttcatagaaagagagtgctgatgtgtgtgaactttcctatataagtgataaacaacatgatttaccatgaggagttccacacccgacgagttgatactattcatagtttttaaagtctgtttgagaggctagaacaagacgaggaattggcaatataatcagaagcaaaaggctagattatttttagcaacacatatgcttttgttccagagtcagcatgtatggcaaaagttccttaggacaattcaatattgaaaatcattatcacttaatataagatgtaatagctaatttatcaatttttcataaaccttcgttgattcaggtaaaggataaatatccattgttcttccaaattctctgaaaaattaaagactttgaataagaactttaaactaataatatatcttgcttattcccgtgctggtaagaagtttgcagaatgaaagtgagccctggtcagagcagcagactcggtttttgttgttttctcaggatattctgattgagagagagcaggacggttgtactcagaatagaaaactagagggtttccaatatacatggtaatgtttctaaactaatctttcccaagtcacaagaaactatcgtttcagctaggatcaaactctaaggtgctatgtggttttttttccctatattagagagcatggatagaatgataatgagattaatgtaagaacttaaaaggaaaagaagattgatgtttaagggttagaaggaatttaggggtgtggtagagatatcatcctttcttggtttttatggtaattatgggtctaatccatttttttaatcttgccaatcacatacttagaaaataatgcatttttaaaaaatagtaattatatgttaaatgaaccacaacatcattctgtttagaagccagagaactaattggctcttgcactgtgtattttatttactgttgtgtgtatgtttcacatgcatgggaatatgcaaatatgtagagaccagagcagcacatagggcatcttctatagcgctccgcctttctgccttgagacaggcatgaatggtaacctcactgtctgtttgtgagctcttcatatccacctgactctcttcctcaatgctgggattataggcatgcacaattacggccttctttttaagaaagtggtaaagatttgaacttatatcctcatgcctgctaggcaaatgatcttaaccactgtaccatcttgtccatttaatgcatttcatatttcatacagaaaaatagtatattccaatatattgaaatcaggtattggtgcttgttagcacttaaaagacagaattcagtgtgaacatgctgacatggaatgagaagggggaagaaatatattctgacattcctttcataatcatgccaagcataaccctgtttatgcttcctatagccctggaatgagaccagatgtgagttctcctccgtccagctcctccacagcaacgggaccacctcccaaactctgcctggtgtgctccgatgaagcttcgggatgccattatggggtgctgacgtgtggaagctgtaaagtcttctttaaaagagcagtggaaggtagtgtgtgttttgaagagtttaatttttgttcctatttctcacagtaaatgtttaactgttcattgtatacaaagtccactaatgatagacattagttcaggtgcaagcatggtggctcttaaatcctccattgggaagggacagaagtaaggagaagaactagaataagtaattaagttcagcctgagaaagaactaaagcaagcattgtaaggaacgctgctgcacggactggcagtacgctgagtctgggcagtccgagaatggtggcttttcattccctgtaagcaagctggcttcaaagctaccagctataccagactgacaaaatcagtgaccctggggaggaacaataagggagggacagcattctttgctttacttttttcctgttaaataaataaagaggtcatgttaagtaccacaatggctgaggccaaattaacaaagtacagagattatcagataggtgaagtctagaaatatggtgtttgaaagaagatgactgcaaggtgatcatgtgttgacgggttcacattccagagcagccggttaatattagagatagtttatataccctacccaaaagtatcttaatggaattaaaccagaaaataaagctacattgatctttcaatatactacaattcacagcccattgttcttatttaaaatgaactcattcccttatttttatgtaaaaataagtattagctatgctatttataaaatgtttaccatttagttatttcagcaaagcgtggtggctagattacattagtaaggaagcctttagctgtgtacggtgttaatgagtaagcctcttggctcagcttcctccctaacttgagtttcagtgttctcaaaggcactttccagctctaacattctgtaaacccttgatactctaattcaaggaaaaaaatttatctagacttgtaaaatccataacatagtttgtagcatttcatttttaaaattattaaattttatttgatattacatacattacatatataacataacattttaacacatactagaaattatacttaaaccctgtattggcattataaatacccaaacataaatcagagttaagttttacacatttgttatttatataagacaaatctattttttaatccaagatcctatgagttctccatgtccttatgcccagctggtcctttttacttgtattcattttacctacaagtatctataactgtattggtagaccataagtggatagttaccaatgactcttcacttgccgtgtgtgaagtacatgcatccaaaatcaattagtggccctgtgaacacttagatcctgttaggatctgtgtctgccagactagcaaacaggttcagaaagttgaagtagccaagctgggattgcaacaaagtggtctccaagtaaggtcttattctcaacaaagatcctcaaagatgcatctgacaagcccttgatacctgctagattataattcaggtagatgctgaaataaaagtaatcacaagagatttaactattagctactatatactgagtagttatggtgtgttgagcacagtatcgtctgcataattacttccaagcttggcaaattttctaacagttcataagagaacaaactccaaaaagttaatgtttccaagatcagtttcttggcatgccactataccactgctactgagaactgctatagaagcgtaagctgatagtattagtttatacttaatctccaggtactcacagtacagtctgtagcctgcatcttttacatgtgttgtttcccatttcctataccatgttgttctatgcaacattttattggtcataggatccagctttggtatgattctagaaaggattacactacaccttattaggagacagatatcactggctatcctgtaagctggctgctacaccaatcataaatagcaaggcactgtcaagtgctaaaagtagatttagccaaaagcaccaacaaccaacagcattaaagaggctttggatgagctgaaggctcttcccttgacattaagaagttagaaaggaaatttatcaggtggcagaaaacaaaggaattccagacgactgtcttaaaaagacatgtaattggtagtacatggccattctcattctttgtgaaataatgaacactagctcaaatcccctgttttgttacaaggaatgaatagactagtggatcattctattatactaacctgagccctatagctaagattctcagttatactacacactggataaagaaataaatatcagcataatacacagtcaatcttttagttagacataaataaacactgaattttttctactctagaagtaatttttccccagtttttccagcaacactgatataaggattctctgaaagacaatggatgtaggggtgtttttatggggtaaaattttagtgctttaactgtggatacaatctgttgtttctccttttgctgcatgatggaagtttttaaaaagaatttatttatccttatttatatgtataaatgtttacctgtataaacactgtgtgcctggtacctgcagagctaaagggcatctggttccctagaattggagttatagacagttgtgacctgctgccatgtgggtgctaggaactgaaccaaagtcctttgcaagagcagcaagtgttcttaactactgagccatctctccaatcccatataataggattttattgagttagttcttctattttgtgtttaagaaaacaggaggcttataaaatacagaaagtatttgaaagagctagcatagatattaggcatgggaaagtaaccctgaaataaagccattatataacttaactatatagctaagtgtatatatatatatatatatatatatatatatatatatatatatatatatatatatacacacacatacatacatatgactattagtcaacttcgtaactgtatcttcacaaaagattagactaatacagttaactatgtactccaaactcatatttaaacaacatcatgattttacatatcaggaaaaactaaatgttttttttttaatccaagtcttagattgccctaataaagaaaaaagtctcttcttagaaatcaggtctatatatgttccaactggtgaactgactgttacaattctttctcatttctttgaatatgatttattctaatataagtaacacagattgttaattactggtgtatctatatagacagttaaatcttgtgtaggaagcttctataccccaccccagtcaaatagatccttcagttgttttcatcaacatttaatactcagattaaagaagacagtgcatgtgggaaacagaaacacttttagccttttcctttttaacagtcttagaattttttgccaggagagaactgaagttcataatatgttcttcagtagtggataccagaaaatggagccactagctagcattaagtcagtatttgaggccaaaggtgactccatgcatctttgtattctaaaagtatattaaataatgatctttgttagtttgtttagtttgccacatttgtgatttacctaaccagtggctagaagttggtaacattttacttcatttaaaattgggaagaacttcttttacctacacatgataatgttgagtgatgaattaaagtcatctttttccttagggattccaaaagaaaatttaagcctgcttccttttagtcacttcaacaatagatcagaattcataataaaagtaataattgcaaaccccatttataactgttaaataactatagaaagcaggttttaaagtttgatcttagagtattcttatgatttgaatacatatatatatgaaatactgtagttgggttgttttcattgattttaaattcgttaagcagaacaagtcctctcaacattgtaattcatgcagataaacagacttaacatccttagtcagtgactttgtgtcttgatgatagtctgctcaaaacaaaaacagaaaggggataggctttttcttttaaaatatccttacagatctgtggaattttaacaatgcccttttaatctcttataggacagcacaattacctttgtgctggaagaaatgattgcatcattgataaaattcgaagaaaaaactgtccagcatgccgctatcgaaaatgtcttcaagctggaatgaacctggaaggtaatggaagcttaaggaactgtctgcctgtcacatgagaactgatcctttactttataaaagctagcaagtgaaccatgtaatgaactctatatatgcttaattagcaaaatgttatattaaaatggttttttccacgatcatatcattttgtgtgttaattcaactacttttgttatatacattgaatttgggacagccatggtgtatattaagtaaagagattacataatattgaaaattacataacttggagcaattttgttttcatccttcttactccttttttaaaactcaaaaccatttcctaagattggaaaagtattgtacatgagatctagttcatattcctctcttactttgcacttcaaaaaccaccacaccttttacttgaaaacatatgttttattacaactctgccaacaacattggatgggttgttttatggtttaagtataaaggaaaagtaaaaactacttgtaagttataatttgatattcctgcttatataaaaagttatataattaacacttatttaaaaacttccctctggaaacgtcagggttttgctgtagtggaaatagcaagctctgtgaaagtagcattcttcctaactcatgttgttagtattgactgttactgtggtacatcatagcaaacgctcagcaaatataaagagaatacaaatttagtacctgagtctgagcagcttactttctgtttatgctacagtgtcaatcacaaaatagtatggcactctacaaaggcaatactattacaggaccaagacggaaggccatctgagcctgtcctgaaagtcagactcatcctggaagaagtagcactgagctgcaaatacagaccttgaggaggaattagatagccatgacagagacgtgctcatagttgtactttataaagagcagtctttgggatagacaaatggatcggtggcaggggatccaacttcacttctcagcacccttatttgctgagaactcacagctgcctctaactgggaattgaactcagcatctctagaagagcggtcagtgctcttaactgctgagccatttctccagcctgagactcaaaagtttaaggtgaacagtaaactcttggttccctgttagaggaaacaagtcctgggagggaattcctatagagaatcctggaattgggagttccatgggtaaaatcacttgctaggcacgcatgaaagacccaagttaggatcccactacccacttaaaaagccaggtctagctatgctcttgcctattaaccacagaattttgaaggataaggtggagacaggagtgtttctagggcttactggcctacctccaagtccaacgagagagcctgcctcaaaagagtgatagacttggatacccaacttcttcccctggccttgagtcatgcacagatgtgtgcatatgcacgtgtacatacaccacactcacacatgtacacacagattcacatagacatgccaaaagttttaaatagtttacttctttcaataatagtgtcagaaattaaagacctcttataacttgaaagtataggctacaaagttgatttcgtatatttttttcttttttcaatattcacagctcgaaaaacgaagaaaaaaattaaaggaattcagcaagccactgcaggagtctcacaagacacttctgaaaacgctaacaaaacaatagttcctgccgcgctgccacagcttacccctaccctggtgtcactgctggaggtgatcgagcctgaggtgttatatgcaggatatgacagctctgttccagactcagcatggagaattatgaccacgctcaacatgttaggtgggcgccaagtgattgccgcagtgaaatgggcaaaggcgataccaggtaaggcatgaaggatgagccacggcataaatctttgtgttttattgaataaatgcactctgagttgggcttggtgaataacttcgtgacatgccatctgcttatcatacacaatccttggggtcagtctgcagcaccctcggaatagagctgctctgagactctaggcggtctgtcctcctgacggagctcacggcagagctggtttcttttactcacaaggacaaaagagagatccaaagtatactggatactgttccctgactttcaggttacacatagtgtctcattttacataagatcttatattatccgaactaaaattccattcacagcgacacctggaagacagcatttagaaattccatttattttcattcagattgtttttgaagttgataaacagcaggttttgttgactttgtcaaaatttactaacacaaagttattttaatttttcattaaatgaaatggcatctactgactataaactttattctagctttaaaggtatttttaaacaagtttcatgttggaacttcgggtgcagcagaggccaaggttaagcatccgcccttactttacagatagcaaagtggttggcacgtttatggctgcatacacctaagctgccatcaccccacctttttagcaaaagaagatggatgtcaggaaattttaaaaatctaaatctgttaagaaataggaatcctaagcaaagactctcggctttctcttttactatatattctactttgtgtgcacttaaatggacaaaattggccaaaaaatagctattcataagtgtttttaaatcattaaagagggttgagtgcccaggcgttgatctggtcaatggctgtctactaaagatcacatccaaacaaggccaacttgtcaactcgagtgttccttatacctttatttcccatctaggattcagaaacttacacctggatgaccaaatgacccttctacagtactcatggatgtttctcatggcatttgccctgggttggagatcatacagacaagcaagtggaaacctgctatgctttgctcctgatctgattattaatgagtaagttacatggccttaaccctccacaaagaactatagcaactaagcagtgctgacaacaggagaaatggttttccccagagactaatacatcagttggttatccagtaccattggtcatccctagaagcatgcatacaagtaacatcatgcatactgagcaggtttcacttatgtatttaggaatatatatatgaatatacatgcatgaatgtaacagcaattcatgcacaaagtggccatcaatttgaaggagagcaaaaagaggaatgtgaaaaggtttagagagaggaaagggaagggaaggggcaaatgactttgtaatctgagaaaaatgaaagaattttaaaataaataagcagactaaaagacctgtaaaaaataactaatccctaatgtttccataaaagtatgctgtaaagattccagaattagatagcttattgagttctgagtatataaaggattcacaattttgttgtttcaaaacagcaaaaacctagtgtcacatgtaaagggttattattgaatagtctctccatgttctccaacattctagttctataataggttattgggaatagcagtaggtatgtttgcagccatgacttcataaatttaaaaaccaacccaaaattcagctaatattttagactagcactatttctttctaactcacatctgtatttgtgtctcccaggcagagaatgactctaccctgcatgtatgaccaatgtaaacacatgctgtttatctccactgaattacaaagattgcaggtatcctatgaagagtatctctgtatgaaaaccttactgcttctctcctcaggttggtggaccacctactcacttttaactttcattctaaatatggcaaatgcatgtgaaagttgtataactattgtgcatatatgtgtatatatatgtatgtgtgtgtgtatatatatatatatatatatatatatatatatatatatatgccaatttcattgtgttttcctagtaagatgatttttagaaaggcaaactgtaaaaagggtatatatatatattaagtgttatcaatattagtaaaataaataggtattgtagatctaggtgtgggatctgcccagggatctctggtgatgaggctccatatagagtcttccttcctgctacccattcatctatatctatataagatgtggagaaagaatgccaacagtaatgagtctgtctctctactgtacgagtgtttctagataaacagtgacagctaccatgaatggggaatagctgagaaaataatcacagtttgtttttcagaacaaccaaatgtttaaactttagactttagcaagactactttatattaaaagtaaaaaagttcaaaaaattaaagaatcttgtctatattagttaagtaataaaaggaaagtcatacctgatattctcttaaaatatacaaagatctcttcaagtcagcaattgaaacaacccagtttttaaggactgggaatatgcaaagatgacttattaaataagatagaataaagacagtaagtctgtcacattccataaggacatgtctagaatgagccaatccagagtagtgagtacaccaaatactggttgaggacacagagtacaggaattctttttcactgctgaaagaatagcaaagtggaatgaccatttcagaagacagtttggtagtttcctataaaggcgaacatagtctcaccatctggtgtagcaattgcttgatcagtatacaaaggaaatgaaaacttatgcagcatggatatttatagccaagctgtttgtatctggctcaacttaggcgcaatgagatatatcatccagtagacaaatcaataaactgacatatgcaaatgatggtattactcaacactgaaaagaaatgggctgttgtgccaataaaaaaatattattgaatcttaaatatatactattaaaaaaagtaatttcttaaaaagaatctcatcaactctaagaaacagatggaggcagatgagatgacttgtgtaaaatggaagctcaacatgcccagcaagccaactttcctcagaaattttacaggtgcttgtggtgaagataagaggaaaactctcccgtttttatgtcagttagaaagggggataaactgttggaggtacatgtagtatttccaaagcaaaagcaaggcctgccctataagagaacctcatcaagaccttatctgacatggagcaaaggcaaataaaggattctagacccttataatagccctcctgacttcacagggaagaaaaggggaaaatgggtttccaggaagcatttgggtgcagtaaaagtgaaatgtaatcttaagagaaaacatttcattggagccttgcttacagttcccagagcttagcttagttcattatcgtgatggcaggcaagcatgattctggaaaagtagctgagtgctacactctgatcggcaagcagagagagaaagagagtagcactgggcctgttgtggacttttgagacctcaaagcttatcctacagtgtcacacagtaacagggccacacctactcaaataagagcacacctcccaatccttctaatcctttcatagctcccctccctggtaactaagcctatgggaccattcttattcaaaccaccacagtgcagataaccaacattaaacacagtccagagcctagccagattaccatactatcttccaccaaatgtcttaattccatcagttctgatactccttgtctagctttcaataaaaaacaaagccagcattaaatctgccccaaaccactggcattatcacattggaatttaaaataactgcaatatttttgctaaggacactacaggaaaaataaagaccacatgagaattatgaatgaagagtcatagaagctaaggaggttcctctaagggagcactagagataacagatgctgaaatgataatgcacagtgctcctagcaggcagaccatcagttgtcatataaccaagaaagagtgagcatagggcaacaaaaacctcctagactcaaataaaaagagggaaaagaataaaaaaaaggtgtagagagatcataaaaaatattttaagtaataaaagttcaaaaatttctaaaattagttatatgtaccaaaatgtggatgcaggatgctcaaggataaccaagcaaagaaaaacaataacaacaaaaaaatctatattttggcatatcctcttcaaattataaaaaaaaaaaagattttttaccctgagttatggttttaaaacagcaatgacaacaaccagacttcttatcagaaatcatgcaagtaagaatgtgaagtaaatagttagaaatgaaagcaaacaacccaccagtcctgagacaatagatccaacagaattacctctcagaagtgaggaagatggtgccatgcacagtagtagacacctagaattctagcactccagagactaaggactgaaattcaagccagccatggctacctatcaagactgtctcaagaaataaagtaatgggtaaaataaaaacattctcaaacaaatgtgtctttttaaagggattttgtggtcatctttacatctgtcctacatgagctattttaaaaattgttcagaagagaggaaaatatgtcagaattatgtgctgcataagaaagagattatagctgggcagtggtggcgcacgcctttaatcccggcacttgggaggcagaggcaggtggatttctgagttcgagaccagcctggtctacagagttccaggacagccagggctacacagagaaaccctgtctcggaaaaagaaagaaagaaaaggaaggaaagaaggaagggagggagggagggagggattttaatagaaaagggatgcctgagagtaaaatgtcaattttcttcttaatgttacataactgttatagtgataataatcaggtatcaggggatctggagaggtggctcagcagttcccagcacccacataaccactcagaactgtctaactcaggtcccagtgatccattgcctacttctaacttctggggtacatacttgtggtgtacaaacatacatcaggcaaaacactcatgcacacagatacacacacacacacacacacacacacacacttttatttatcttacaaattaacaatcgcagactatgtgattgaaatacaggcatatgtacaatgaatgacagaggtattgtagttaagttaggagggaaatttcagatacactctggcattgactttgcctgttagagtagttctagtgtatttcggaagggagaacaggttagttgtaaatgtgtactgcagatgacagagcagtcactaagagttcaaactgtcaaagggaacacgcaatcatgagcatgtctgggacactgaaggctgatgggagtctccagagtctagtggttgaaagtgtgcaccacaggcagggtccccagcttccccaaacctgatgtgcttggtcagaacagaagcacctctcggggccaagctaagggggaagctccttcccctcctcggggcaaagttggaaaaactgctaagtgtgagcaaaggcaatccccagaaacaaaaaagcaaaatggattcttggtgcaaaactagtagcaagagcacaggtctgcttcagtgagctccttggggctgtttctaaaatgtgttcatgggaggcacatctgctaaggaatgaaaaactgaagacaacaagaaagaggaatggaaaccagcatttcctcctagggagattaagttcagtataatggagaaagaaggaaggagaagaaagcctggaaggagaatgacagacttaattccctggtgacactgcagacgctggggagatgtgcggcagcaatgatacaatctagggaaagcctgctgaacggaagaagaaggacagcgagagttgtgcccacagcgataagggagacaggttcagtaaaaaggacagctgtgaagctttcaaatcagaggaagatgattaataatgaagtggatctccttcagtgtgttcttgtcaatgaagtatttaaacaactcccttcaaagaaaaagactgaaatcaaaggctctatagaagtgcttctaaactctctagccgtttgctttgtaatagttaacacacagtacagacaccctttcctggaggtactgacctcacctggtacagtatgaaggttgtaaaatggaagattgtttaaagcgggttctcactggtacacatcacaaattagctatacacaggatgacacggtttgcatcctcgggtgtcgtcaccgctcccgttataagatgggagacatttggatctcccatggttatgaaccccaattctcagttagcctatactttcacttagctatagtcccaccatcctttgagaatgcatgaattttagagtgtattgaatgtacattgtaacgatgaaatacgcatgaaatgtgctacttagtacaagacaagttaaaaaaaggaaaatttaactgacttcatcttaccttttagttcctaaggaaggtctgaagagccaagagttatttgatgagattcgaatgacttatatcaaagagctaggaaaagccattgtcaaaagggaaggaaactccagtcagaattggcagcggttttatcaactgacaaaacttttggactccatgcatgatgtaagtaccaaacataaatccaaaacaacagcaaaaaagaaaaagtaggttggggtggtggcagatgcctttaatcacagcagacacagaggtagagcaagtagatgtctatgagtctgaggctgctctacatagggaatgccaggacagtcagagatacgtaagagatcctatctcaaaagcccaaagacagagacagagagagacagagacagaagaacattgtaggtcctaggttatggtttaattagtgtatgtgtatagttatataccaatcagtcattatagtgattattcaagataaataattttttacacatttctcttaagtcttactagtctggtcatttgcgtaaagatgactctgttagtacagagttcagtagctcctgattttaagtatccctgaagtacatgaatgagctatcagattacactagtttcacctccctgtgtatcagtactggtgcagtctcctttgagcctgctgcctcgtctcctgactgctgggatcacaggcatgccaggcttcattctgcacacagtttaagatgccttttgttcttatgtgcttattttatttaaattattttcttatctgaggaattccaacaaagattggtatatttgtgatcaaattgttaaaatggccagtagggcaggaatgcaggatctatataccaggtcttctgtgtgtatattacgactttcagtttagtgtgtctgggattctggagtgtgggaaccagtaggtccctgactcttgttcctgttcttgggttctcctttttctctttgtcttttccaacaatatgatggtttttattttattttgctatgttttattttattatcacctcttagaagcacaatctttttaataagaggagaagttgagaggaactgggagggtaagccacaatcaagatacattacatgagaaaagaatttattttcagtaaacaaaaaaatatgtcaagacttggtgtggggagccgagagatatgagccattcgcagttatgctttgaagcctcaccattaccgtatcttctcttgcaggtggttgaaaatctccttagctactgcttccaaacatttttggataagtccatgagtattgaattcccagagatgttagctgaaatcatcactaatcagataccaaaatactcaaatggaaatatcaaaaagcttctgtttcatcagaaatgactgccttactaagaaaggctgccttaaagaaagttgaatttatagcttttactgtacaaacttatcaacttgtcttgtagatgttttgtcgttctttttgtttgtcttgtttgttttctatacgcactacatgtggtctctagagggccaagacttggcaacagaagcagatgagccatcacttttcagtgacaggaaagcagacagtgatgtgcattggctggtgtatcacagaaactagaacagttagtggagacatgtccactatcagagaaggaccgcacctgaaccaccagtgcccaaagtccatgtgatcaactttctgctcaactttcagttggctggataacactttctagacttttctgttggtgtatttttcccatgtatagttaggatagcattttgatttatgcatggaaacctgaaaaaagtttacacgtgtatatcagaaaagggaagttgtgccttttatagctattactgtctggttttaacaatttcctttatattcagtgaactatgcttgctcgtttttcttaaataatttttgtattctagttattgtatagctgtttaagatgggcagctgcctcacagctctcctagacgctaacattaatttccgtgtgaaaatgggtcggtgctcctaccctgatggcactcagctatcagaagaccacagaaattgactcagatctccagtattcttgtcaaaagctcttactctgtatatatctgcttccatggggaattatataggttgtgcagattaaccgtcctaactggtatagagcacctagtccagtgacctgctgggtaaactgtggatgatggttacaaaagactaattgtaaaacagtgcccaccaacaggccccgtttgcacccaatgcaccatctcttcagtggtgcgatagcaacaaagtttgtaactcagctctttcaggaccttcgggagtagtttgtgtaacattttaaaatgtattattccagataaccagctgtgataaagccgagagattgttttaatcagaccaagtaacttctctcattaaacgttaccctcaactaagtctctaatatggcaagaatggctagacacccattttcacatcccacctgtcaccaattggtctagctttcctggtggtacaggaaaatcagctactgattttttgttatttagaactgaatgtcaggcatccatgtttgtccaactatacatccctacatgtgccatagaatctaacacaagtcttgtgaacttcttcacactgagagttatcattttaaacaaaacagaagctgtagtagccctttctgtgtgcaccttaccaactttctgtgactcaaagcttaacacacttactaagccacaagaaatctgatttctacttaaggtggccaaattatttgtgtaatagaaaactgaaaatctaatattaaaaatatgaaacttctaatatatttttatatttagttctagtttcagatatatatcatattggtattcactaatctgggaagggaagggctactgcagctgtacatgcaatttattaacatgattgtaaaatagctgtatagtgtaaaataagaatgatttttagatgagattgttttatcatgacatgttatatattttttgtaggggtcaaagaaatgttgatggatatcctataagatttatagtatataagagcatccatacaggcctcagtggtcttggaaattaaaacaggtttgctctaagctagggagagggagctgggactggccctgtgtgcagtgcaggtcctgagggtttgacccgatcagatcacaggggaactaattccctcccatctaaccatcctcatccgaccatggccctgtcagtgcaggctggctttattaaatccaggacagaaaggtggcgcttatgtacttagaggcaccgtccagtaacagggttgttcccacatgcagcctccgcacgggttaacagaaacagaggctttagaagtttggcaataatgtgcatagaggttccagcaatatgtaaatactaaagaatcgcataggaagccaataatacactaatcctctccatcctacaagagtccatttccaagtaagatgaggacatgtttatgttttctttgaatgctttttgaatgttgttattttcagtattttgcagaaattatttaataaaaaaaagtataatcatttgctttttgaattctctctaaaagggaatgttcagtttgtaatggtttaaattggtctcaaagtactttaaaataattgtaacccagctggatgtgaaatttatggtgcctaagaaataccacttgaagattatcaatgacagtgttaagtttcaaaatgagcttctcaaaaatagattattgtacatttatggaatgttatatggttaaacccaaaaagcacatcacacataaatctgctttcagttccaaccagcttggctttcaaaaatagagctccaaaaaaaaaaaaggaaaaaaaagatatatatgctttgttattaacagaaggcagcagacattcataaaactactatcggaagttttccattagatgtataaagagctatcctttggtatgtgggaaagaagaaagctgtcataattctgattgagtataagtgagagagatacggtactgtttgagagcagctccttttctgcgtgtggcttcataccgttccaaactatgtagattttataatagcttcagtgagaattggtaacatgcctgtatgactcacaacagatcttgaaaactatctttaattactggtaggacaaaaagggacattctggttattttaggcactggcttggaacactgtatatgcagaagaaagaagacaggcaatctggggaaaggaaggggacctgggaagcactgccttctttaaggaaagacacaccaatagatgagatcatcccaaaggcacagggaccacagagtgtgagtccttagtgacgagtcaggtgagctctg

SEQ ID NO: 221 - DNA, 20 bp, Artificial Sequence
  Antisense Compound
cattggcaaatattaacttc

SEQ ID NO: 222 - DNA, 20 bp, Artificial Sequence
  Antisense Compound
tttggagtccattggcaaat

SEQ ID NO: 223 - DNA, 20 bp, Artificial Sequence
  Antisense Compound
tttctgaatcctggtatcgc

SEQ ID NO: 224 - DNA, 20 bp, Artificial Sequence
  Antisense Compound
tccttaggaactgaggagag

SEQ ID NO: 225 - DNA, 20 bp, Artificial Sequence
  Antisense Compound
taaggcagtcatttctgatg

SEQ ID NO: 226 - DNA, 20 bp, Artificial Sequence
  Antisense Compound
aaggcagcctttcttagtaa

SEQ ID NO: 227 - DNA, 20 bp, Artificial Sequence
  Antisense Compound
aagtttgtacagtaaaagct

SEQ ID NO: 228 - DNA, 20 bp, Artificial Sequence
  Antisense Compound
gctcatctgcttctgttgcc

SEQ ID NO: 229 - DNA, 20 bp, Artificial Sequence
  Antisense Compound
gcatacatactgtgagcccg

SEQ ID NO: 230 - DNA, 20 bp, Artificial Sequence
  Antisense Compound
ctgggcggccccgtctgcag

SEQ ID NO: 231 - DNA, 20 bp, Artificial Sequence
  Antisense Compound
ttggcaaatattaatgtgag

SEQ ID NO: 232 - DNA, 20 bp, Artificial Sequence
  Antisense Compound
agccagataaacaagtcggc

SEQ ID NO: 233 - DNA, 20 bp, Artificial Sequence
  Antisense Compound
atattaactcagcaccggcg

SEQ ID NO: 234 - DNA, 20 bp, Artificial Sequence
  Antisense Compound
agaatcttagctatagggct

SEQ ID NO: 235 - DNA, 20 bp, Artificial Sequence
  Antisense Compound
catgccttacctggtatcgc

SEQ ID NO: 236 - DNA, 20 bp, Artificial Sequence
  Antisense Compound
tgtaacttactcattaataa

SEQ ID NO: 237 - DNA, 20 bp, Artificial Sequence
  Antisense Compound
tcacatagtctgcgattgtt

SEQ ID NO: 238 - DNA, 20 bp, Artificial Sequence
  Antisense Compound
tccttaggaactaaaaggta

SEQ ID NO: 239 - DNA, 20 bp, Artificial Sequence
  Antisense Compound
tatctctgactgtcctggca

SEQ ID NO: 240 - DNA, 20 bp, Artificial Sequence
  Antisense Compound
agcctttcttagtaaggcag

SEQ ID NO: 241 - DNA, 20 bp, Artificial Sequence
  Antisense Compound
acatcactgtctgctttcct

SEQ ID NO: 242 - DNA, 20 bp, Artificial Sequence
  Antisense Compound
ggacatgtctccactaactg

SEQ ID NO: 243 - DNA, 20 bp, Artificial Sequence
  Antisense Compound
tttgggcactggtggttcag

SEQ ID NO: 244 - DNA, 20 bp, Artificial Sequence
  Antisense Compound
catcttaaacagctatacaa

SEQ ID NO: 245 - DNA, 20 bp, Artificial Sequence
  Antisense Compound
ctgatagctgagtgccatca

SEQ ID NO: 246 - DNA, 20 bp, Artificial Sequence
  Antisense Compound
agagatggtgcattgggtgc

SEQ ID NO: 247 - DNA, 20 bp, Artificial Sequence
  Antisense Compound
cctgacattcagttctaaat

SEQ ID NO: 248 - DNA, 20 bp, Artificial Sequence
  Antisense Compound
caaacatggatgcctgacat

SEQ ID NO: 249 - DNA, 20 bp, Artificial Sequence
  Antisense Compound
ttagattctatggcacatgt

SEQ ID NO: 250 - DNA, 20 bp, Artificial Sequence
  Antisense Compound
gttaagctttgagtcacaga

SEQ ID NO: 251 - DNA, 20 bp, Artificial Sequence
  Antisense Compound
ctctccctagcttagagcaa

SEQ ID NO: 252 - DNA, 20 bp, Artificial Sequence
  Antisense Compound
tggacggtgcctctaagtac

SEQ ID NO: 253 - DNA, 20 bp, Artificial Sequence
  Antisense Compound
gaaatggactcttgtaggat

SEQ ID NO: 254 - DNA, 20 bp, Artificial Sequence
  Antisense Compound
ataaatttcacatccagctg

SEQ ID NO: 255 - DNA, 20 bp, Artificial Sequence
  Antisense Compound
taaatgtacaataatctatt

SEQ ID NO: 256 - DNA, 1552 bp, Artificial Sequence
  R. norvegicus
atggactccaaagaatccttagctccccctggtagagacgaagtccctggcagtttgcttggccaggggagggggagcgtaatggacttttataaaagcctgaggggaggagctacagtcaaggtttctgcatcttcgccctcagtggctgctgcttctcaggcagattccaagcagcagaggattctccttgatttctcgaaaggctccacaagcaatgtgcagcagcgacagcagcagcagcagcagcagcagcagcagcagcagcagcagcagcagcagcagcagccagacttatccaaagccgtttcactgtccatggggctgtatatgggagagacagaaacaaaagtgatggggaatgacttgggctacccacagcagggccaacttggcctttcctctggggaaacagactttcggcttctggaagaaagcattgcaaacctcaataggtcgaccagcgttccagagaaccccaagagttcaacgtctgcaactgggtgtgctaccccgacagagaaggagtttcccaaaactcactcggatgcatcttcagaacagcaaaatcgaaaaagccagaccggcaccaacggaggcagtgtgaaattgtatcccacagaccaaagcacctttgacctcttgaaggatttggagttttccgctgggtccccaggtaaagacacaaacgagagtccctggagatcagacctgttgatagatgaaaacttgctttctcctttggcgggagaagatgatccattccttctcgaaggggacacgaatgaggattgtaagcctcttattttaccggacactaaacctaaaattaaggatactggagatacaatcttatcaagtcccagcagtgtggcactgccccaagtgaaaacagaaaaagatgatttcattgaactttgcacccccggggtaattaagcaagagaaactgggcccagtttattgtcaggcaagcttttctgggacaaatataattggtaataaaatgtctgccatttctgttcatggtgtgactacctctggaggacagatgtaccactatgacatgaatacagcatccctttctcagcagcaggatcagaagcctgtttttaatgtcattccaccaattcctgttggttctgaaaactggaataggtgccaaggctccggagaggacagcctgacttccttgggggctctgaacttcccaggccggtcagtgttttctaatgggtactcaagccctggaatgagaccagatgtaagctctcctccatccagctcgtcagcagccacgggaccacctcccaagctctgcctggtgtgctccgatgaagcttcaggatgtcattacggggtgctgacatgtggaagctgcaaagtattctttaaaagagcagtggaaggacagcacaattacctttgtgctggaagaaacgattgcatcattgataaaattcgaaggaaaaactgcccagcatgccgctatcggaaatgtcttcaggctggaatgaaccttgaagctcgaaaaacaaagaaaaaaaaaaaaa

SEQ ID NO: 257 - DNA, 20 bp, Artificial Sequence
  Antisense Compound
taagtctggctgctgctgct

SEQ ID NO: 258 - DNA, 20 bp, Artificial Sequence
  Antisense Compound
ctttggataagtctggctgc

SEQ ID NO: 259 - DNA, 20 bp, Artificial Sequence
  Antisense Compound
aggcttttataaaagtccat

SEQ ID NO: 260 - DNA, 20 bp, Artificial Sequence
  Antisense Compound
atcaaggagaatcctctgct

SEQ ID NO: 261 - DNA, 20 bp, Artificial Sequence
  Antisense Compound
gcccccaaggaagtcaggct

SEQ ID NO: 262 - DNA, 20 bp, Artificial Sequence
  Antisense Compound
ccgtaatgacatcctgaagc

SEQ ID NO: 263 - DNA, 20 bp, Artificial Sequence
  Antisense Compound
actcttggctcttcagacct

SEQ ID NO: 264 - DNA, 20 bp, Artificial Sequence
  Antisense Compound
taaggcagtcatttttgatg

SEQ ID NO: 265 - DNA, 20 bp, Artificial Sequence
  Antisense Compound
aactttctttaaggcaacct

SEQ ID NO: 266 - DNA, 20 bp, Artificial Sequence
  Antisense Compound
cctctataaaccacatgtac

SEQ ID NO: 267 - DNA, 20 bp, Artificial Sequence
  Antisense Compound
tgtcatcacttcagagtgtt

SEQ ID NO: 268 - DNA, 20 bp, Artificial Sequence
  Antisense Compound
aactgttagtttctgtgata

SEQ ID NO: 269 - DNA, 20 bp, Artificial Sequence
  Antisense Compound
tagaaagttttacccagcca

SEQ ID NO: 270 - DNA, 20 bp, Artificial Sequence
  Antisense Compound
ctatgtaattctccatggaa

SEQ ID NO: 271 - DNA, 20 bp, Artificial Sequence
  Antisense Compound
ctggactaggtgctctacac

SEQ ID NO: 272 - DNA, 20 bp, Artificial Sequence
  Antisense Compound
attgaagagatggtgcatta

SEQ ID NO: 273 - DNA, 20 bp, Artificial Sequence
  Antisense Compound
ggctttatcagagctggcta

SEQ ID NO: 274 - DNA, 20 bp, Artificial Sequence
  Antisense Compound
ctgtattagcgatttagttg

SEQ ID NO: 275 - DNA, 20 bp, Artificial Sequence
  Antisense Compound
accatgagagctagaccaat

SEQ ID NO: 276 - DNA, 20 bp, Artificial Sequence
  Antisense Compound
gcacatgtagggatgtgtag

SEQ ID NO: 277 - DNA, 20 bp, Artificial Sequence
  Antisense Compound
agtttttctattacacaaat

SEQ ID NO: 278 - DNA, 20 bp, Artificial Sequence
  Antisense Compound
cagtagccctttccctttcc

SEQ ID NO: 279 - DNA, 20 bp, Artificial Sequence
  Antisense Compound
catcaatatttctttgaccc

SEQ ID NO: 280 - DNA, 20 bp, Artificial Sequence
  Antisense Compound
aatggactattgaagggtgg

SEQ ID NO: 281 - DNA, 20 bp, Artificial Sequence
  Antisense Compound
agaaaacataagcatgtcct

SEQ ID NO: 282 - DNA, 20 bp, Artificial Sequence
  Antisense Compound
gaacaatcccttttagagag

SEQ ID NO: 283 - DNA, 20 bp, Artificial Sequence
  Antisense Compound
taatctatttttgagaagct

SEQ ID NO: 284 - DNA, 20 bp, Artificial Sequence
  Antisense Compound
tacgcttcaaggaaagcttc

SEQ ID NO: 285 - DNA, 20 bp, Artificial Sequence
  Antisense Compound
ccgagtctcactgaagttat

SEQ ID NO: 286 - DNA, 20 bp, Artificial Sequence
  Antisense Compound
tctttcaagatcggtcatga

SEQ ID NO: 287 - DNA, 20 bp, Artificial Sequence
  Antisense Compound
ccaaggcctaaaataaccag

SEQ ID NO: 288 - DNA, 20 bp, Artificial Sequence
  Antisense Compound
ctttgggtactctcacttat

SEQ ID NO: 289 - DNA, 20 bp, Artificial Sequence
  Antisense Compound
cctgactcatccttagaccc

SEQ ID NO: 290 - DNA, 20 bp, Artificial Sequence
  Antisense Compound
tctcaagctccatgatcctt

SEQ ID NO: 291 - DNA, 20 bp, Artificial Sequence
  Antisense Compound
cgccttctaacactgaaacc

SEQ ID NO: 292 - DNA, 20 bp, Artificial Sequence
  Antisense Compound
ctgtttcgccttctaacact

SEQ ID NO: 293 - DNA, 20 bp, Artificial Sequence
  Antisense Compound
gtttgggaatgagaagactt

SEQ ID NO: 294 - DNA, 20 bp, Artificial Sequence
  Antisense Compound
tagcagctggtcaccagtcc

SEQ ID NO: 295 - DNA, 20 bp, Artificial Sequence
  Antisense Compound
attttcatacagccatttat

SEQ ID NO: 296 - DNA, 20 bp, Artificial Sequence
  Antisense Compound
tattgacacactgaaatctc

SEQ ID NO: 297 - DNA, 20 bp, Artificial Sequence
  Antisense Compound
tagaaagacggatttttaaa

SEQ ID NO: 298 - DNA, 20 bp, Artificial Sequence
  Antisense Compound
tgtggtttggtaataccaag

SEQ ID NO: 299 - DNA, 20 bp, Artificial Sequence
  Antisense Compound
actaacatttactgccaatt

SEQ ID NO: 300 - DNA, 20 bp, Artificial Sequence
  Antisense Compound
gttcattctaaagtggtcac

SEQ ID NO: 301 - DNA, 20 bp, Artificial Sequence
  Antisense Compound
aatattcgcaccccactggt

SEQ ID NO: 302 - DNA, 20 bp, Artificial Sequence
  Antisense Compound
cgttattaacctccgttgaa

SEQ ID NO: 303 - DNA, 20 bp, Artificial Sequence
  Antisense Compound
ttctacctcgcgcgatttac

SEQ ID NO: 304 - DNA, 20 bp, Artificial Sequence
  Antisense Compound
ctgctagcctctggatttga

SEQ ID NO: 305 - DNA, 20 bp, Artificial Sequence
  Antisense Compound
ccttccctgaaggttcctcc

SEQ ID NO: 306 - DNA, 20 bp, Artificial Sequence
  Antisense Compound
cgacctattgaggtttgcaa

SEQ ID NO: 307 - DNA, 20 bp, Artificial Sequence
  Antisense Compound
gtcaaaggtgctttggtctg

SEQ ID NO: 308 - DNA, 20 bp, Artificial Sequence
  Antisense Compound
gcagacattttattaccaat

SEQ ID NO: 309 - DNA, 20 bp, Artificial Sequence
  Antisense Compound
gtacatctgtcctccagagg

SEQ ID NO: 310 - DNA, 20 bp, Artificial Sequence
  Antisense Compound
gctgtattcatgtcatagtg

SEQ ID NO: 311 - DNA, 19 bp, Artificial Sequence
  Synthetic oligonucleotide
cgagaggcggacgggaccg

SEQ ID NO: 312 - DNA, 21 bp, Artificial Sequence
  Synthetic oligonucleotide
cgagaggcggacgggaccgtt

SEQ ID NO: 313 - DNA, 21 bp, Artificial Sequence
  Synthetic oligonucleotide
ttgctctccgcctgccctggc

SEQ ID NO: 314 - DNA, 19 bp, Artificial Sequence
  Synthetic oligonucleotide
gctctccgcctgccctggc

(19) United States
(12) Patent Application Publication
(10) Pub. No.: US 2013/0143943 A1
(43) Pub. Date: Jun. 6, 2013

(54) MODULATION OF GLUCOCORTICOID RECEPTOR EXPRESSION

(71) Applicant: Isis Pharmaceuticals, Inc., Carlsbad, CA (US)

(72) Inventors: Sanjay Bhanot, Carlsabd, CA (US); Susan M. Freier, Carlsbad, CA (US); Kenneth W. Dobie, Del Mar, CA (US)

(73) Assignee: Isis Pharmaceuticals, Inc., Carlsbad, CA (US)

(21) Appl. No.: 13/650,801

(22) Filed: Oct. 12, 2012

Related U.S. Application Data
(63) Continuation of application No. 11/039,629, filed on Jan. 20, 2005, now abandoned.
(60) Provisional application No. 60/538,173, filed on Jan. 20, 2004.
(60) Provisional application No. 60/550,191, filed on Mar. 3, 2004.

(51) Int. Cl. C07H 21/04

(52) U.S. Cl. CPC C07H 21/04
USPC 514/44 A; 536/24.5; 435/375

(57) ABSTRACT

Compounds, compositions and methods are provided for modulating the expression of glucocorticoid receptor. The compositions comprise oligonucleotides, targeted to nucleic acid encoding glucocorticoid receptor. Methods of using these compounds for modulation of glucocorticoid receptor expression and for diagnosis and treatment of diseases and conditions associated with expression of glucocorticoid receptor are provided.

### SEQUENCE LISTING

The present application is being filed along with a Sequence Listing in electronic format. The Sequence Listing is provided as a file entitled GCCRSEQ.txt created Oct. 12, 2012, which is 152 Kb in size. The information in the electronic format of the sequence listing is incorporated herein by reference in its entirety.

### FIELD OF THE INVENTION

The present invention provides compositions and methods for modulating the expression of glucocorticoid receptor. In particular, this invention relates to antisense compounds, particularly oligonucleotide compounds, which, in preferred embodiments, hybridize with nucleic acid molecules encoding glucocorticoid receptor. Such compounds are shown herein to modulate the expression of glucocorticoid receptor.

### BACKGROUND OF THE INVENTION

Glucocorticoids were among the first steroid hormones to be identified and are responsible for a multitude of physiological functions, including the stimulation of gluconeogenesis, decreased glucose uptake and utilization in peripheral tissues, increased glycogen deposition, suppression of immune and inflammatory responses, inhibition of cytokine synthesis and acceleration of various developmental events. Glucocorticoids are also especially important for combating stress. Stress-induced elevation of glucocorticoid synthesis and release leads to, among other responses, increased ventricular workload, inhibition of inflammatory mediators, inhibition of cytokine synthesis and increased glucose production (Karin, Cell, 1998, 93, 487-490).

Both natural glucocorticoids and their synthetic derivatives exert their action through the glucocorticoid receptor, a ubiquitously expressed cytoplasmic member of the nuclear hormone superfamily of receptors. Complementary DNA clones encoding the human glucocorticoid receptor (also known as nuclear receptor subfamily 3, group C, member 1; NR3C1; GCCR; GCR; GRL; Glucocorticoid receptor, lymphocyte) were first isolated in 1985 (Hollenberg et al., Nature, 1985, 318, 635-641; Weinberger et al., Science, 1985, 228, 740-742). The gene is located on human chromosome 5q11-q13 and consists of 9 exons (Encio and Detera-Wadleigh, J Biol Chem, 1991, 266, 7182-7188; Gehring et al., Proc Natl Acad Sci USA, 1985, 82, 3751-3755). Multiple forms of human glucocorticoid receptor mRNA exist: a 5.5 kb human glucocorticoid receptor a cDNA containing exons 1-8 and exon 9α; a 4.3 kb human glucocorticoid receptor β cDNA containing exons 1-8 and exon 9β; and a 7.0 kb human glucocorticoid receptor α cDNA containing exons 1-8 and the entire exon 9, which includes exon 9α, exon 9β and the ‘J region’, which is flanked by exons 9α and 9β (Hollenberg et al., Nature, 1985, 318, 635-641; Oakley et al., J Biol Chem, 1996, 271, 9550-9559). Human glucocorticoid receptor α is the predominant isoform of the receptor and the one that exhibits steroid binding activity (Hollenberg et al., Nature, 1985, 318, 635-641). Additionally, through usage of three different promoters three different exons 1 can be transcribed, and alternative splicing of one exon 1 variant can result in three different versions of this exon. Thus, human glucocorticoid receptor mRNA may contain 5 different versions of exon 1 (Breslin et al., Mol Endocrinol, 2001, 15, 1381-1395).

Examination of the expression patterns of the α and β isoforms of human glucocorticoid receptor mRNA reveals that the α isoform is more abundantly expressed. Both isoforms are expressed in similar tissues and cell types, including lung, kidney, heart, liver, skeletal muscle, macrophages, neutrophils and peripheral blood mononuclear cells. Only human glucocorticoid receptor α is expressed in colon. At the level of protein, while the α isoform is detected in all tissues examined, the β isoform is undetectable, suggesting that under physiological conditions, the default splicing pathway is the one that produces the α isoform (Pujols et al., Am J Physiol Cell Physiol, 2002, 283, C1324-1331). The β isoform of glucocorticoid receptor binds neither a glucocorticoid agonist nor an antagonist. Furthermore, the β isoform is localized primarily in the nucleus in transfected cells, independent of hormone stimulation. When both isoforms are expressed in the same cell, the glucocorticoid receptor β inhibits the hormone-induced, glucocorticoid receptor α-mediated stimulation of gene expression, suggesting that the β isoform functions as an inhibitor of glucocorticoid receptor a activity (Oakley et al., J Biol Chem, 1996, 271, 9550-9559). Unless otherwise noted, the human glucocorticoid receptor described herein is defined as the ubiquitous product(s) of the gene located on chromosome 5q11-q13.

The human glucocorticoid receptor is comprised of three major domains, the N-terminal activation domain, the central DNA-binding domain and the C-terminal ligand-binding domain (Giguere et al., Cell, 1986, 46, 645-652). In the absence of ligand, the glucocorticoid receptor forms a large heteromeric complex with several other proteins, from which it dissociates upon ligand binding. The heat shock protein 90 (hsp90) performs a key role in this complex, keeping the receptor in a conformation capable of binding to steroid by incapable of activating transcription (Cadepond et al., J Biol Chem, 1991, 266, 5834-5841). The glucocorticoid receptor is phosphorylated in the absence of ligand, and becomes hyperphosphorylated after the binding of an agonist, such as a steroid, but not an antagonist, such as the antiglucocorticoid compound RU-486 (Orti et al., J Biol Chem, 1989, 264, 9728-9731).

The phosphorylated glucocorticoid receptor subsequently translocates to the nucleus through the action of two domains which participate in nuclear localization, NL1, localized in the region bridging the DNA-binding and ligand-binding domains and NL2, localized completely within the ligand-binding domain. The function of NL1 is inhibited by the ligand-binding domain, and this inhibition can be abrogated by ligand binding (Picard and Yamamoto, Embo J, 1987, 6, 3333-3340). Nuclear translocation occurs in a hormone-dependent manner.

Once activated, the glucocorticoid receptor forms a homodimer. Studies of the purified activated glucocorticoid receptor demonstrate that it exists as a homodimer in the presence and absence of DNA, suggesting that dimerization occurs before DNA binding (Wrange et al., J Biol Chem, 1989, 264, 5253-5259). The dimerized glucocorticoid receptor binds to specific palindromic DNA sequences named glucocorticoid-responsive elements (GREs) in its target genes, and consequently affects transcription (Schaaf and Cidlowski, J Steroid Biochem Mol Biol, 2002, 83, 37-48). The regulatory regions of the tyrosine aminotransferase, alanine aminotransferase and phosphoenolpyruvate carboxykinase (PEPCK) genes, among others, contain positive GREs, which serve to enhance transcription. In addition to activating transcription following binding to positive GREs, the glucocorticoid receptor can also repress transcription through binding to negative GREs, which represses transcription, or through transcription interference via interactions of the glucocorticoid receptor with other transcription factors (Karin, Cell, 1998, 93, 487-490). The latter is a DNA-binding independent activity. Thus, the glucocorticoid receptor can influence transcription through both DNA-independent and DNA-dependent mechanisms. While the glucocorticoid receptor gene is itself essential for survival, as demonstrated by the lack of viability in glucocorticoid receptor-deficient mice, the DNA binding activity of the glucocorticoid receptor is not essential for survival. Mice bearing a point mutation in the glucocorticoid receptor that impairs dimerization and consequently GRE-dependent transactivation are viable, revealing the in vivo relevance of the DNA-binding-independent activities of the glucocorticoid receptor (Reichardt et al., Cell, 1998, 93, 531-541).

Owing to the ubiquitous expression of the glucocorticoid receptor, and to its ability to both activate and repress transcription, the glucocorticoid receptor often requires cofactors to confer transcriptional specificity. Certain cofactors facilitate transcription through the recruitment of the basal transcription machinery or the remodeling of chromatin. The CREB-binding protein (CBP) and its homolog p300 function as coactivators for the glucocorticoid receptor, enhancing transcription of glucocorticoid receptor responsive genes (Chakravarti et al., Nature, 1996, 383, 99-103). Another class of coactivators include the vitamin D receptor-interacting proteins (DRIP) DRIP150 and DRIP205, both of which facilitate glucocorticoid receptor transcriptional activation (Hittelman et al., Embo J, 1999, 18, 5380-5388). Human glucocorticoid receptor also associates with the chromatin remodeling complex BRG, which removes histone H1 from chromatin and allows general transcription factors to access their binding sites (Fryer and Archer, Nature, 1998, 393, 88-91). In this case, the glucocorticoid receptor appears to recruit the BRG complex to promoters via interactions with the BRG-associated factor BAF250, a subunit of the BRG complex. Once escorted to the promoter, BRG induces chromatin remodeling and transcription proceeds (Deroo and Archer, Oncogene, 2001, 20, 3039-3046; Nie et al., Mol Cell Biol, 2000, 20, 8879-8888). A transcription factor whose activity is negatively regulated by the glucocorticoid receptor is NF-kB. Dexamethasone, a ligand of the glucocorticoid receptor, promotes the binding of the glucocorticoid receptor to the p65 subunit of NFkB, which inhibits the activation of the interleukin-6 promoter (Ray and Prefontaine, Proc Natl Acad Sci USA, 1994, 91, 752-756).

Cell lines transfected with a complementary glucocorticoid receptor antisense RNA strand exhibited a reduction in glucocorticoid receptor mRNA levels and a decreased response to the glucocorticoid receptor agonist dexamethasone (Pepin and Barden, Mol Cell Biol, 1991, 11, 1647-1653). Transgenic mice bearing an antisense glucocorticoid receptor gene construct were used to study the glucocorticoid feedback effect on the hypothalamus-pituitary-adrenal axis (Pepin et al., Nature, 1992, 355, 725-728). In another study of similarly genetically engineered mice, energy intake and expenditure, heart and vastus lateralis muscle lipoprotein lipase activity, and heart and brown adipose tissue norepinephrine were lower than in control animals. Conversely, fat content and total body energy were significantly higher than in control animals. These results suggest that a defective glucocorticoid receptor system may affect energy balance through increasing energetic efficiency, and they emphasize the modulatory effects of hypothalamic-pituitary-adrenal axis changes on muscle lipoprotein lipase activity (Richard et al., Am J Physiol, 1993, 265, R146-150).

Behavorial effects of glucocorticoid receptor antagonists have been measured in animal models designed to assess anxiety, learning and memory. Reduced expression of glucocorticoid receptor in rats long-term intracerebroventricularly infused with antisense oligodeoxynucleotides targeting glucocorticoid receptor mRNA did not interfere with spatial navigation in the Morris water maze test (Engelmann et al., Eur Pharmacol, 1998, 361, 17-26). Bilateral infusion of an antisense oligodeoxynucleotide targeting the glucocorticoid receptor mRNA into the dentate gyrus of the rat hippocampus reduced the immobility of rats in the Porsolt forced swim test (Korte et al., Eur J Pharmacol, 1996, 301, 19-25).

Glucocorticoids are frequently used for their immunosuppressive, anti-inflammatory effects in the treatment of diseases such as allergies, athsma, rheumatoid arthritis, AIDS, systemic lupus erythematosus and degenerative osteoarthritis. Negative regulation of gene expression, such as that caused by the interaction of glucocorticoid receptor with NF-kB, is proposed to be at least partly responsible for the anti-inflammatory action of glucocorticoids in vivo. Interleukin-6, tumor necrosis factor α and interleukin-1 are the three cytokines that account for most of the hypothalamic-pituitary-adrenal (HPA) axis stimulation during the stress of inflammation. The HPA axis and the systemic sympathetic and adrenomedullary system are the peripheral components of the stress system, responsible for maintaining basal and stress-related homeostasis. Glucocorticoids, the end products of the HPA axis, inhibit the production of all three inflammatory cytokines and also inhibit their effects on target tissues, with the exception of interleukin-6, which acts synergistically with glucocorticoids to stimulate the production of acute-phase reactants. Glucocorticoid treatment decreases the activity of the HPA axis (Chrousos, N Engl J Med, 1995, 332, 1351-1362).

In some cases, patients are refractory to glucocorticoid treatment. One reason for this resistance to steroids lies with mutations or polymorphisms present in the glucocorticoid receptor gene. A total of 15 missense, three nonsense, three frameshift, one splice site, and two alternative spliced mutations, as well as 16 polymorphisms, have been reported in the NR3C1 gene in association with glucocorticoid resistance (Bray and Cotton, Hum Mutat, 2003, 21, 557-568). Additional studies in humans have suggested a positive association between metabolic syndrome incidence and progression, with alleles at the glucocorticoid receptor (GR) gene (Rosmond, Obes Res, 2002, 10, 1078-1086).

Other cases of glucocorticoid insensitivity are associated with altered expression of glucocorticoid receptor isoforms. A study of human glucocorticoid receptor β isoform mRNA expression in glucocorticoid-resistant ulcerative colitis patients revealed the presence of this mRNA was significantly higher than in the glucocorticoid-sensitive patients, suggesting that the expression of human glucocorticoid receptor β mRNA in the peripheral blood mononuclear cells may serve as a predictor of glucocorticoid response in ulcerative colitis (Honda et al., Gastroenterology, 2000, 118, 859-866). Increased expression of glucocorticoid receptor β is also observed in a significantly high number of glucocorticoid-insensitive asthmatics. Additionally, cytokine-induced abnormalities in the DNA binding capacity of the glucocorticoid receptor were found in peripheral blood mononuclear cells from glucocorticoid-insensitive patients transfection, and HepG2 cells with the glucocorticoid receptor gene resulted in a significant reduction of glucocorticoid receptor a DNA-binding capacity (Leung et al., J Exp Med, 1997, 186, 1567-1574). Dexamethasone binding studies demonstrate that human glucocorticoid receptor β does not alter the affinity of glucocorticoid receptor a for hormonal ligands, but rather its ability to bind to the GRE (Bamberger et al., J Clin Invest, 1995, 95, 2435-2441). Taken together, these results illustrate that glucocorticoid receptor β, through competition with glucocorticoid receptor a for GRE target sites, may function as a physiologically and pathophysiologically relevant endogenous inhibitor of glucocorticoid action.

In the liver, glucocorticoid agonists increase hepatic glucose production by activating the glucocorticoid receptor, which subsequently leads to increased expression of the gluconeogenic enzymes phosphoenolpyruvate carboxykinase (PEPCK) and glucose-6-phosphatase. Through gluconeogenesis, glucose is formed through non-hexose precursors, such as lactate, pyruvate and alanine (Link, Curr Opin Investig Drugs, 2003, 4, 421-429). Steroidal glucocorticoid receptor antagonists such as RU 486 have been tested in rodent models of diabetes. Mice deficient in the leptin receptor gene, termed db/db mice, are genetically obese, diabetic and hyperinsulinemic. Treatment of hyperglycemic db/db mice with RU 486 decreased blood glucose levels by approximately 49%, without affecting plasma insulin levels. Additionally, RU 486 treatment reduced the expression of glucocorticoid receptor responsive genes PEPCK, glucose-6-phosphatase, glucose transporter type 2 and tyrosine aminotransferase in db/db mice as compared to untreated animals (Friedman et al., J Biol Chem, 1997, 272, 31475-31481). RU 486 also ameliorates diabetes in the ob/ob mouse model of diabetes, obesity and hyperinsulinemia, through a reduction in serum insulin and blood glucose levels (Gettys et al., Int J Obes Relat Metab Disord, 1997, 21, 865-873).

As increased gluconeogenesis is considered to be the major source of increased glucose production in diabetes, a number of therapeutic targets for the inhibition of hepatic glucose production have been investigated. Due to the ability of antagonists of the glucocorticoid receptor to ameliorate diabetes in animal models, such compounds are among the potential therapies being explored. However, there are detrimental systemic effects of glucocorticoid receptor antagonists, including activation of the HPA axis (Link, Curr Opin Investig Drugs, 2003, 4, 421-429). Increased HPA axis activity is associated with suppression of immune-related inflammatory action, which can increase susceptibility to infectious agents and neoplasms. Conditions associated with suppression of immune-mediated inflammation through defects in the HPA axis, or its target tissues, include Cushing's syndrome, chronic stress, chronic alcoholism and melancholic depression (Chrousos, N Engl J Med, 1995, 332, 1351-1362). Thus, it is of great value to develop liver-specific glucocorticoid receptor antagonists. Steroidal glucocorticoid receptor antagonists have been conjugated to bile acids for the purpose of targeting them to the liver (Apelqvist et al., 2000). Currently, there are no known therapeutic agents that target the glucocorticoid receptor without undesired peripheral effects (Link, Curr Opin Investig Drugs, 2003, 4, 421-429). Consequently, there remains a long felt need for agents capable of effectively inhibiting hepatic glucocorticoid receptor.

The U.S. Pat. No. 6,649,341 discloses antisense primers targeted to a nucleotide sequence comprising human glucocorticoid receptor 1 Ap/e transcript, as well as a method of preventing apoptosis in neurons by expressing an antisense transgene to the human glucocorticoid receptor exon 1A transcripts (Vedeckis and Breslin, 2003).

The US Pre-grant publication 20030092616 and the PCT publication WO 02/096943 disclose a nucleotide sequence encoding human glucocorticoid receptor, as well as an antisense oligonucleotide complementary to said polynucleotide; a ribozyme which inhibits STAT6 activation by cleavage of an RNA comprising said polynucleotide; and a method for treating a disease, which comprises administering to a subject an amount of an antisense oligonucleotide or a ribozyme effective to treat a disease selected from the group consisting of allergic disease, inflammation, autoimmune diseases, diabetes, hyperlipidemia, infectious disease and cancers (Honda et al., 2002).

The PCT publication WO 88/00975 discloses an antisense oligonucleotide targeted to a nucleic acid sequence encoding human glucocorticoid receptor.

The PCT publication WO 01/42307 discloses antisense oligonucleotides targeted to a nucleic acid sequence encoding human glucocorticoid receptor.

The PCT publication WO 01/77344 discloses an antisense oligonucleotide targeted to a nucleic acid sequence encoding human glucocorticoid receptor.

The PCT publication WO 03/008583 discloses a carcinoma cancer inhibitor which is an antisense molecule including antisense or sense oligonucleotides comprising a single-stranded nucleic acid sequence (either RNA or DNA) capable of binding to target mRNA (sense) or DNA (antisense) sequences for carcinoma cancer molecules, including a nucleic acid sequence encoding human glucocorticoid receptor (Morris and Engelhard, 2003).

Antisense technology is an effective means of reducing the expression of specific gene products and therefore is uniquely useful in a number of therapeutic, diagnostic and research applications for the modulation of glucocorticoid receptor expression. Furthermore, liver is one of the tissues in which the highest concentrations of antisense oligonucleotides are found following administration (Geary et al., Curr. Opin. Investig. Drugs, 2001, 2, 562-573). Therefore, antisense technology represents an attractive method for the liver-specific inhibition of glucocorticoid receptor. In addition to diabetes, particularly type 2 diabetes, glucocorticoid receptor modulators are useful to treat diseases such as obesity, Metabolic syndrome X, Cushing's Syndrome, Addison's disease, inflammatory diseases such as asthma, rhinitis and arthritis, allergy, autoimmune disease, immunodeficiency, anorexia, cachexia, bone loss or bone frailty, and wound healing. Metabolic syndrome, metabolic syndrome X or simply Syndrome X refers to a cluster of risk factors that include obesity, dyslipidemia, particularly high blood triglycerides, glucose intolerance, high blood sugar and high blood pressure. Scott, C. L., Am J. Cardiol. 2003 Jul. 3; 92(1A):35i-42i. Glucocorticoid receptor inhibitors such as the compounds described herein are also believed to be useful for amelioration of hyperglycemia induced by systemic steroid therapy.

Moreover, antisense technology provides a means of inhibiting the expression of the glucocorticoid receptor β isoform, demonstrated to be overexpressed in patients refractory to glucocorticoid treatment.

The present invention provides compositions and methods for inhibiting glucocorticoid receptor expression.

### SUMMARY OF THE INVENTION

The present invention is directed to antisense compounds, especially nucleic acid and nucleic acid-like oligomers, which are targeted to a nucleic acid encoding glucocorticoid receptor, and which modulate the expression of glucocorticoid receptor. Pharmaceutical and other compositions comprising the compounds of the invention are also provided. Further provided are methods of screening for modulators of glucocorticoid receptor and methods of modulating the expression of glucocorticoid receptor in cells, tissues or animals comprising contacting said cells, tissues or animals with one or more of the compounds or compositions of the invention. Methods of treating an animal, particularly a human, suspected of having or being prone to a disease or condition associated with expression of glucocorticoid receptor are also set forth herein. Such methods comprise administering a therapeutically or prophylactically effective amount of one or more of the compounds or compositions of the invention to the person in need of treatment.

### DETAILED DESCRIPTION OF THE INVENTION

### A. Overview of the Invention

The present invention employs antisense compounds, preferably oligonucleotides and similar species for use in modulating the function or effect of nucleic acid molecules encoding glucocorticoid receptor. This is accomplished by providing oligonucleotides which specifically hybridize with one or more nucleic acid molecules encoding glucocorticoid receptor. As used herein, the terms “target nucleic acid” and “nucleic acid molecule encoding glucocorticoid receptor” have been used for convenience to encompass DNA encoding glucocorticoid receptor, RNA (including pre-mRNA and mRNA or portions thereof) transcribed from such DNA, and also cDNA derived from such RNA. The hybridization of a compound of this invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of glucocorticoid receptor. In the context of the present invention, “modulation” and “modulation of expression” mean either an increase (stimulation) or a decrease (inhibition) in the amount or levels of a nucleic acid molecule encoding the gene, e.g., DNA or RNA. mRNA is often a preferred target nucleic acid. Inhibition is often the preferred form of modulation of expression; it is understood that “inhibition” does not have to be absolute inhibition, but is intended to mean a decrease or reduction in target expression.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present invention comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some preferred embodiments, homology, sequence identity or complementarity, between the oligomeric and target is between about 50% to about 60%. In some embodiments, homology, sequence identity or complementarity, is between about 60% to about 70%. In preferred embodiments, homology, sequence identity or complementarity, is between about 70% and about 80%. In more preferred embodiments, homology, sequence identity or complementarity, is between about 80% and about 90%. In some preferred embodiments, homology, sequence identity or complementarity, is about 90%, about 92%, about 94%, about 95%, about 96%, about 97%, about 98%, about 99% or about 100%.

### B. Compounds of the Invention

According to the present invention, antisense compounds include antisense oligomeric compounds, antisense oligonucleotides, ribozymes, external guide sequence (EGS) oligonucleotides, alternate splicers and other oligomeric compounds which hybridize to at least a portion of the target nucleic acid and modulate its expression. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

The antisense compounds of the present invention also include modified compounds in which a different base is present at one or more of the nucleotide positions in the compound. For example, if the first nucleotide is an adenosine, modified compounds may be produced which contain thymidine, guanosine or cytidine at this position. This may be done at any of the positions of the antisense compound. These compounds are then tested using the methods described herein to determine their ability to inhibit expression of glucocorticoid receptor mRNA.

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the antisense compounds of this invention, the present invention comprehends other families of antisense compounds as well, including but not limited to oligonucleotide analogs and mimetics such as those described herein.

The antisense compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the antisense compounds of the invention are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the antisense compounds of the invention are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

Particularly preferred compounds are oligonucleotides from about 12 to about 50 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well. Exemplary preferred antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). It is also understood that preferred antisense compounds may be represented by oligonucleotide sequences that comprise at least 8 consecutive nucleobases from an internal portion of the sequence of an illustrative preferred antisense compound, and may extend in either or both directions until the oligonucleotide contains about 8 to about 80 nucleobases. One having skill in the art armed with the preferred antisense compounds illustrated herein will be able, without undue experimentation, to identify further preferred antisense compounds.

### C. Targets of the Invention

“Targeting” an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the present invention, the target nucleic acid encodes glucocorticoid receptor.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding glucocorticoid receptor, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which the preferred antisense compounds hybridize are hereinbelow referred to as “preferred target segments.” As used herein the term “preferred target segment” is defined as at least an 8-nucleobase portion of a target region to which an active antisense compound is targeted. While not wishing to be bound by theory, it is presently believed that these target segments represent portions of the target nucleic acid which are accessible for hybridization.

One of skill in the art will recognize that the active antisense compound sequences and their target segments (“preferred target segments”) serve to illustrate and describe particular embodiments within the scope of the present invention. Additional active antisense compounds and preferred target segments may be identified by one having ordinary skill.

Once one or more target regions, segments or sites have been identified, antisense compounds are chosen which are sufficiently complementary to the target, i.e., hybridize sufficiently well and with sufficient specificity, to give the desired effect.

The oligomeric antisense compounds may also be targeted to regions of the target nucleobase sequence (e.g., such as those disclosed in Example 13 and other examples herein) comprising nucleobases 1-80, 81-160, 161-240, 241-320, 321-400, 401-480, 481-560, 561-640, 641-720, 721-800, 801-880, 881-960, 961-1040, 1041-1120, 1121-1200, 1201-1280, 1281-1360, 1361-1440, 1441-1520, 1521-1600, 1601-1680, 1681-1760, 1761-1840, 1841-1920, 1921-2000, 2001-2080, 2081-2160, 2161-2240, 2241-2320, 2321-2400, 2401-2480, 2481-2560, 2561-2640, 2641-2720, 2721-2800, 2801-2880, 2881-2960, 2961-3040, 3041-3120, 3121-3200, 3201-3280, 3281-3360, 3361-3440, 3441-3520, 3521-3600, 3601-3680, 3681-3760, 3761-3840, 3841-3920, 3921-4000, 4001-4080, 4081-4160, 4161-4240, 4241-4320, 4321-4400, 4401-4480, 4481-4560, 4561-4640, 4641-4720 or 4721-4788 of SEQ ID NO: 4, or any combination thereof.

In one embodiment of the present invention, antisense compounds are targeted to nucleotides 13-119 in the 5′ UTR, nucleotides 114-151 in the start codon region, nucleotides 351-533, 667-845, 877-1243, 1356-1488, 1552-1756, 1819-1999, 2008-2139, 2146-2194, 2201-2301, or 2386-2416 in the coding region or nucleotides 2488-2685, 2723-3435, 3499-3789, 3826-3860, 3886-3905, 3918-3937, 4031-4072, 4082-4193 or 4244-4758 in the 3′ UTR, all of SEQ ID NO: 4; or nucleotides 104562-104648 in the 3′ UTR of SEQ ID NO: 25.

In another embodiment of the present invention, antisense compounds are targeted to nucleotides 2-20 in the start codon region, 301-1405, 1459-2043 or 2050-2309 in the coding region, nucleotides 2376-2433 or 2521-2546 in the 3′ UTR, all of SEQ ID NO: 11; nucleotides 227-297 in the 5′ UTR of SEQ ID NO: 219; or nucleotides 14909-18389 in the 3′ UTR of SEQ ID NO: 220.

In a further embodiment of the present invention, antisense compounds are targeted to nucleotides 150-2129 or 2136-2395 in the coding region, or nucleotides 2472-3705, 4576-4867, 5039-5293, 5680-5877 or 6214-6263 in the 3′ UTR, all of SEQ ID NO: 18; or nucleotides 278-304 in the coding region of SEQ ID NO: 256.

### D. Screening and Target Validation

In a further embodiment, the “preferred target segments” identified herein may be employed in a screen for additional compounds that modulate the expression of glucocorticoid receptor. “Modulators” are those compounds that decrease or increase the expression of a nucleic acid molecule encoding glucocorticoid receptor and which comprise at least an 8-nucleobase portion which is complementary to a preferred target segment. The screening method comprises the steps of contacting a preferred target segment of a nucleic acid molecule encoding glucocorticoid receptor with one or more candidate modulators, and selecting for one or more candidate modulators which decrease or increase the expression of a nucleic acid molecule encoding glucocorticoid receptor. Once it is shown that the candidate modulator or modulators are capable of modulating (e.g. either decreasing or increasing) the expression of a nucleic acid molecule encoding glucocorticoid receptor, the modulator may then be employed in further investigative studies of the function of glucocorticoid receptor, or for use as a research, diagnostic, or therapeutic agent in accordance with the present invention.

The preferred target segments of the present invention may be also be combined with their respective complementary antisense compounds of the present invention to form stabilized double-stranded (duplexed) oligonucleotides. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The antisense compounds of the present invention can also be applied in the areas of drug discovery and target validation. The present invention comprehends the use of the compounds and preferred target segments identified herein in drug discovery efforts to elucidate relationships that exist between glucocorticoid receptor and a disease state, phenotype, or condition. These methods include detecting or modulating glucocorticoid receptor comprising contacting a sample, tissue, cell, or organism with the compounds of the present invention, measuring the nucleic acid or protein level of glucocorticoid receptor and/or a related phenotypic or chemical endpoint at some time after treatment, and optionally comparing the measured value to a non-treated sample or sample treated with a further compound of the invention. These methods can also be performed in parallel or in combination with other experiments to determine the function of unknown genes for the process of target validation or to determine the validity of a particular gene product as a target for treatment or prevention of a particular disease, condition, or phenotype.

### E. Kits, Research Reagents, Diagnostics, and Therapeutics

The antisense compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The antisense compounds of the invention are useful for research and diagnostics, because these compounds hybridize to nucleic acids encoding glucocorticoid receptor and modulate the expression of glucocorticoid receptor. The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of glucocorticoid receptor is treated by administering antisense compounds in accordance with this invention. For example, in one non-limiting embodiment, the methods comprise the step of administering to the animal in need of treatment, a therapeutically effective amount of a glucocorticoid receptor inhibitor. The glucocorticoid receptor inhibitors of the present invention effectively inhibit the activity of the glucocorticoid receptor protein or inhibit the expression of the glucocorticoid receptor protein. In one embodiment, the activity or expression of glucocorticoid receptor in an animal is inhibited by about 10%. Preferably, the activity or expression of glucocorticoid receptor in an animal is inhibited by about 30%. More preferably, the activity or expression of glucocorticoid receptor in an animal is inhibited by 50% or more. Thus, the oligomeric antisense compounds modulate expression of glucocorticoid receptor mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of glucocorticoid receptor may be measured in serum, adipose tissue, liver or any other body fluid, tissue or organ of the animal. Preferably, the cells contained within said fluids, tissues or organs being analyzed contain a nucleic acid molecule encoding glucocorticoid receptor protein and/or the glucocorticoid receptor protein itself.

The antisense compounds of the invention can be utilized in pharmaceutical compositions by adding an effective amount of a compound to a suitable pharmaceutically acceptable diluent or carrier. Use of the compounds and methods of the invention may also be useful prophylactically.

The compounds of the present inventions are inhibitors of glucocorticoid receptor expression. Thus, the compounds of the present invention are believed to be useful for treating metabolic diseases and conditions, particularly diabetes, hyperglycemia induced by systemic steroid therapy, obesity, hyperlipidemia or metabolic syndrome X. The compounds of the present invention may also be useful for treating Cushing's Syndrome, Addison's disease, allergy, autoimmune disease, immunodeficiency, anorexia, cachexia, bone loss or bone frailty, for treating inflammatory diseases such as asthma, rhinitis and arthritis, and for promoting wound healing. The compounds of the invention are also believed to be useful for preventing or delaying the onset of metabolic diseases and conditions, particularly diabetes, obesity, hyperlipidemia or metabolic syndrome X, and for preventing or delaying the onset of Cushing's Syndrome, Addison's disease, allergy, autoimmune disease, immunodeficiency, anorexia, cachexia, bone loss, or inflammatory diseases such as asthma, rhinitis and arthritis.

The compounds of the invention have been found to be effective for lowering blood glucose, including plasma glucose, and for lowering blood lipids, including serum lipids, particularly serum cholesterol and serum triglycerides. The compounds of the invention are therefore particularly useful for the treatment, prevention and delay of onset of type 2 diabetes, high blood glucose and hyperlipidemia. Surprisingly, the compounds of the invention have been found to have these therapeutic effects in the absence of certain side effects such as, for example, elevated corticosterone levels or lymphopenia which are associated with systemic inhibition of glucocorticoid receptor signaling.

### F. Modifications

As is known in the art, a nucleoside is a base-sugar combination. The base portion of the nucleoside is normally a heterocyclic base sometimes referred to as a “nucleobase” or simply a “base”. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Modified Internucleoside Linkages (Backbones)

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkyl-phosphotriaminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphos-phonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

### Modified Sugar and Internucleoside Linkages-Mimetics

In other preferred antisense compounds, e.g., oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Preferred embodiments of the invention are oligonucleotides with phosphorothioate backbones and oligonucleosides with heteroatom backbones, and in particular —CH₂—NH—O—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

### Modified Sugars

Modified antisense compounds may also contain one or more substituted sugar moieties. Preferred are antisense compounds, preferably antisense oligonucleotides, comprising one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage can be a methylene (—CH₂—) group bridging the 2′ oxygen atom and the 4′ carbon atom, for which the term LNA is used for the bicyclic moiety. LNAs and preparation thereof are described in WO 98/39352 and WO 99/14226. In the case of an ethylene group in this position, the term ENA is used (Singh et al., Chem. Commun., 1998, 4, 455-456; ENA™: Morita et al., Bioorganic Medicinal Chemistry, 2003, 11, 2211-2226).

### Natural and Modified Nucleobases

Antisense compounds may also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

### Conjugates

Another modification of the antisense compounds of the invention involves chemically linking to the antisense compound one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include intercalators, reporter molecules, polyamines, polyamides, polyethylene glycols, poly-ethers, groups that enhance the pharmacodynamic properties of oligomers, and groups that enhance the pharmacokinetic properties of oligomers. Typical conjugate groups include cholesterols, lipids, phospho-lipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Antisense compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

### Chimeric Compounds

It is not necessary for all positions in a given compound to be uniformly modified, and in fact more than one of the aforementioned modifications may be incorporated in a single compound or even at a single nucleoside within an oligonucleotide.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. Chimeric antisense oligonucleotides are thus a form of antisense compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

### G. Formulations

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety. For oligonucleotides, presently preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Sodium salts are presently believed to be more preferred.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. application Ser. Nos. 09/108,673 (filed Jul. 1, 1998), 09/315,298 (filed May 20, 1999) and 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of antisense compounds and other non-antisense drugs are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Alternatively, compositions of the invention may contain two or more antisense compounds targeted to different regions of the same nucleic acid target. Numerous examples of antisense compounds are known in the art. Two or more combined compounds may be used together or sequentially.

### H. Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.0001 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.0001 ug to 100 g per kg of body weight, once or more daily, to once every 20 years.

While the present invention has been described with specificity in accordance with certain of its preferred embodiments, the following examples serve only to illustrate the invention and are not intended to limit the same. Each of the references, GenBank accession numbers, and the like recited in the present application is incorporated herein by reference in its entirety.

### EXAMPLES

### Example 1

### Synthesis of [2′-O-(2-Methoxyethyl)]-[2′-deoxy]-[2′-O-(Methoxyethyl)] Chimeric Phosphorothioate Oligonucleotides

[2′-O-(2-methoxyethyl)]-[2′-deoxy]-[2′-O-(methoxyethyl)]chimeric phosphorothioate oligonucleotides were prepared as per the procedure described in U.S. Patent Application Ser. Nos. 60/538,173 and 60/550,191, the contents of which are herein incorporated by referenece in their entirety, for the 2′-O-methyl chimeric oligonucleotide, with the substitution of 2′-O-(methoxyethyl) amidites for the 2′-O-methyl amidites.

### [2′-O-(2-Methoxyethyl)Phosphodiester]-[2′-deoxy Phosphorothioate]—[2′-O-(2-Methoxyethyl) Phosphodiester]Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per as per the procedure described in U.S. Patent Application Ser. Nos. 60/538,173 and 60/550,191, the contents of which are herein incorporated by referenece in their entirety, for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,1′-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 2

### Design and Screening of Duplexed Antisense Compounds Targeting Glucocorticoid Receptor

In accordance with the present invention, a series of nucleic acid duplexes comprising the antisense compounds of the present invention and their complements can be designed to target glucocorticoid receptor. The nucleobase sequence of the antisense strand of the duplex comprises at least an 8-nucleobase portion of an oligonucleotide in Table 1. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO: 311) and having a two-nucleobase overhang of deoxythymidine(dT) would have the following structure:

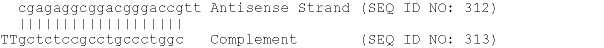

In another embodiment, a duplex comprising an antisense strand having the same sequence CGAGAGGCGGACGGGACCG (SEQ ID NO: 311) may be prepared with blunt ends (no single stranded overhang) as shown:

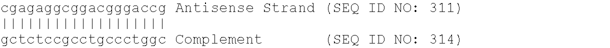

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate glucocorticoid receptor expression. When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 3

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 4

### Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 5

### Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 6

### Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

### T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

### A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

### NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

### HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

### HepG2 Cells:

The human hepatoblastoma cell line HepG2 was obtained from the American Type Culture Collection (Manassas, Va.). HepG2 cells were routinely cultured in Eagle's MEM supplemented with 10% fetal calf serum, non-essential amino acids, and 1 mM sodium pyruvate (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

### b.END Cells:

The mouse brain endothelial cell line b.END was obtained from Dr. Werner Risau at the Max Plank Instititute (Bad Nauheim, Germany). b.END cells were routinely cultured in DMEM, high glucose (Gibco/Life Technologies, Gaithersburg, Md.) supplemented with 10% fetal calf serum (Gibco/Life Technologies, Gaithersburg, Md.). Cells were routinely passaged by trypsinization and dilution when they reached 90% confluence. Cells were seeded into 96-well plates (Falcon-Primaria #3872) at a density of 3000 cells/well for use in RT-PCR analysis.

For Northern blotting or other analyses, cells may be seeded onto 100 mm or other standard tissue culture plates and treated similarly, using appropriate volumes of medium and oligonucleotide.

### NRK Cells:

Normal rat kidney (NRK) cells were obtained from American Type Culture Collection (Manassus, Va.). They were grown in serial monolayer culture in Minimum Essential Media (Invitrogen Life Technologies, Carlsbad, Calif.) supplemented with 10% fetal bovine serum, (Invitrogen Life Technologies, Carlsbad, Calif.), 100 ug/ml penicillin and 100 ug/ml streptomycin and 0.1 mM non-essential amino acids (all supplements from Invitrogen Life Technologies, Carlsbad, Calif.) in a humidified atmosphere of 90% air-10% CO² at 37° C. Cells were routinely passaged by trypsinization and dilution when they reached 85-90% confluencey. Cells were seeded into 96-well plates (Falcon-Primaria #353872, BD Biosciences, Bedford, Mass.) at a density of 6000 cells/well for use in antisense oligonucleotide transfection.

### Primary Mouse Hepatocytes:

Primary mouse hepatocytes are prepared from CD-1 mice purchased from Charles River Labs. Primary mouse hepatocytes are routinely cultured in Hepatocyte Attachment Media (Invitrogen Life Technologies, Carlsbad, Calif.) supplemented with 10% Fetal Bovine Serum (Invitrogen Life Technologies, Carlsbad, Calif.), 250 nM dexamethasone (Sigma-Aldrich Corporation, St. Louis, Mo.), 10 nM bovine insulin (Sigma-Aldrich Corporation, St. Louis, Mo.). Cells are seeded into 96-well plates (Falcon-Primaria #353872, BD Biosciences, Bedford, Mass.) at a density of 4000-6000 cells/well for treatment with the oligomeric compounds of the invention.

Treatment with Antisense Compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTINT™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 1) which is targeted to human H-ras, or ISIS 18078, (GTGCGCGCGAGCCCGAAATC, SEQ ID NO: 2) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 3, a 2′-O-methoxyethyl gapmer (2′-β-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 7

### Analysis of Oligonucleotide Inhibition of Glucocorticoid Receptor Expression

Antisense modulation of glucocorticoid receptor expression can be assayed in a variety of ways known in the art. For example, glucocorticoid receptor mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of glucocorticoid receptor can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to glucocorticoid receptor can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 8

### Design Of Phenotypic Assays for the Use of Glucocorticoid Receptor Inhibitors

### Phenotypic Assays

Once Glucocorticoid Receptor inhibitors have been identified by the methods disclosed herein, the compounds are further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of glucocorticoid receptor in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with glucocorticoid receptor inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the genotype of the cell (measurement of the expression of one or more of the genes of the cell) after treatment is also used as an indicator of the efficacy or potency of the glucocorticoid receptor inhibitors. Hallmark genes, or those genes suspected to be associated with a specific disease state, condition, or phenotype, are measured in both treated and untreated cells.

### Example 9

### RNA Isolation

Poly(A)+mRNA Isolation

Poly(A)+mRNA was isolated according to Miura et al., (Clin. Chem., 1996, 42, 1758-1764). Other methods for poly(A)+mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Total RNA Isolation

Total RNA was isolated using an RNEASY96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY96™ well plate attached to a QIAVAC™ manifold fitted with a waste collection tray and attached to a vacuum source. Vacuum was applied for 1 minute. 500 μL of Buffer RW1 was added to each well of the RNEASY96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 10

### Real-Time Quantitative PCR Analysis of Glucocorticoid Receptor mRNA Levels

Quantitation of glucocorticoid receptor mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and primers to human glucocorticoid receptor were designed to hybridize to a human glucocorticoid receptor sequence, using published sequence information (GenBank accession number NM_{—}000176.1, incorporated herein as SEQ ID NO: 4). For human glucocorticoid receptor the PCR primers were:

forward primer: AGGTTGTGCAAATTAACAGTCCTAACT (SEQ ID NO: 5)
reverse primer: TAGTC=GCAACCATCATCCA (SEQ ID NO: 6) and the PCR probe was: FAM-AGCACCTAGTCCAGTGACCTGCTGGGTAAA-TAMRA
(SEQ ID NO: 7) where FAM is the fluorescent dye and TAMRA is the quencher dye. For human GAPDH the PCR primers were:
forward primer: GAAGGTGAAGGTCGGAGTC(SEQ ID NO: 8)
reverse primer: GAAGATGGTGATGGGATTTC (SEQ ID NO: 9) and the PCR probe was: 5′ JOE-CAAGCTTCCCGTTCTCAGCC-TAMRA 3′ (SEQ ID NO: 10) where JOE is the fluorescent reporter dye and TAMRA is the quencher dye.

Probes and primers to mouse glucocorticoid receptor were designed to hybridize to a mouse glucocorticoid receptor sequence, using published sequence information (GenBank accession number NM_{—}008173.1, incorporated herein as SEQ ID NO: 11). For mouse glucocorticoid receptor the PCR primers were:

forward primer: GACATCTTGCAGGATTTGGAGTT (SEQ ID NO: 12)
reverse primer: AACAGGTCTGACCTCCAAGGACT (SEQ ID NO: 13) and the PCR probe was: FAM-CGGGTCCCCAGGTAAAGAGACAAACGA-TAMRA
(SEQ ID NO: 14) where FAM is the fluorescent reporter dye and TAMRA is the quencher dye. For mouse GAPDH the PCR primers were:
forward primer: GGCAAATTCAACGGCACAGT(SEQ ID NO: 15)
reverse primer: GGGTCTCGCTCCTGGAAGAT(SEQ ID NO: 16) and the PCR probe was: 5′ JOE-AAGGCCGAGAATGGGAAGCTTGTCATC-TAMRA 3′ (SEQ ID NO: 17) where JOE is the fluorescent reporter dye and TAMRA is the quencher dye.

Probes and primers to rat glucocorticoid receptor were designed to hybridize to a rat glucocorticoid receptor sequence, using published sequence information (GenBank accession number NM_{—}012576.1, incorporated herein as SEQ ID NO: 18). For rat glucocorticoid receptor the PCR primers were:

forward primer: AAACAATAGTTCCTGCAGCATTACC (SEQ ID NO: 19)
reverse primer: CATACAACACCTCGGGTTCAATC (SEQ ID NO: 20) and the PCR probe was: FAM-ACCCCTACCTTGGTGTCACTGCT-TAMRA
(SEQ ID NO: 21) where FAM is the fluorescent reporter dye and TAMRA is the quencher dye. For rat GAPDH the PCR primers were:
forward primer: TGTTCTAGAGACAGCCGCATCTT(SEQ ID NO: 22)
reverse primer: CACCGACCTTCACCATCTTGT(SEQ ID NO: 23) and the PCR probe was: 5′ JOE-TTGTGCAGTGCCAGCCTCGTCTCA-TAMRA 3′ (SEQ ID NO: 24) where JOE is the fluorescent reporter dye and TAMRA is the quencher dye.

### Example 11

### Northern Blot Analysis of Glucocorticoid Receptor mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBONDT™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human glucocorticoid receptor, a human glucocorticoid receptor specific probe was prepared by PCR using the forward primer AGGTTGTGCAAATTAACAGTCCTAACT (SEQ ID NO: 5) and the reverse primer TAGTCTTTTGCAACCATCATCCA (SEQ ID NO: 6). To normalize for variations in loading and transfer efficiency membranes were stripped and probed for human glyceraldehyde-3-phosphate dehydrogenase (GAPDH) RNA (Clontech, Palo Alto, Calif.).

To detect mouse glucocorticoid receptor, a mouse glucocorticoid receptor specific probe was prepared by PCR using the forward primer GACATCTTGCAGGATTTGGAGTT (SEQ ID NO: 12) and the reverse primer AACAGGTCTGACCTCCAAGGACT (SEQ ID NO: 13). To normalize for variations in loading and transfer efficiency membranes were stripped and probed for mouse glyceraldehyde-3-phosphate dehydrogenase (GAPDH) RNA (Clontech, Palo Alto, Calif.).

To detect rat glucocorticoid receptor, a rat glucocorticoid receptor specific probe was prepared by PCR using the forward primer AAACAATAGTTCCTGCAGCATTACC (SEQ ID NO: 19) and the reverse primer CATACAACACCTCGGGTTCAATC (SEQ ID NO: 20). To normalize for variations in loading and transfer efficiency membranes were stripped and probed for rat glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 12

### Antisense Inhibition of Human Glucocorticoid Receptor Expression by Chimeric Phosphorothioate Oligonucleotides Having 2′-MOE Wings and a Deoxy Gap

In accordance with the present invention, a series of antisense compounds was designed to target different regions of the human glucocorticoid receptor RNA, using published sequences (GenBank accession number NM_{—000176.1}, incorporated herein as SEQ ID NO: 4, nucleotides 1 to 106000 of the sequence with GenBank accession number AC012634, incorporated herein as SEQ ID NO: 25, GenBank accession number X03348.1, incorporated herein as SEQ ID NO: 26 and GenBank accession number U01351.1, incorporated herein as SEQ ID NO: 27). The compounds are shown in Tables 1 and 2. “Target site” indicates the first (5′-most) nucleotide number on the particular target sequence to which the compound binds. All compounds in Tables 1 and 2 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytosine residues are 5-methylcytosines.

The compounds in Table 1 were analyzed for their effect on human glucocorticoid receptor mRNA levels in T-24 cells by quantitative real-time PCR as described in other examples herein. Data, shown in Table 1, are averages from two experiments in which T-24 cells were treated with 100 nM of the antisense oligonucleotides of the present invention. The positive control for each datapoint is identified in the table by sequence ID number. If present, “N.D.” indicates “no data”.

TABLE 1
Inhibition of human glucocorticoid receptor
mRNA levels by chimeric phosphorothioate
oligonucleotides having 2′-MOE wings and a deoxy gap

| | | TARGET | | | | SEQ | CONTROL |
|---|---|---|---|---|---|---|---|
| | | SEQ ID | TARGET | | % | ID | SEQ |
| ISIS # | REGION | NO | SITE | SEQUENCE | INHIB | NO | ID NO |
| 153080 | Coding | 4 | 2197 | ttgatgtaggtcattctaat | 28 | 30 | 2 |
| 153081 | 3′UTR | 4 | 3875 | tggcttagtaaatatgttaa | 21 | 31 | 2 |
| 153082 | 3′UTR | 4 | 4031 | cttcccttcccagattagtg | 39 | 32 | 2 |
| 153083 | 3′UTR | 4 | 3336 | aaccatcatccacagtttac | 57 | 33 | 2 |
| 153084 | 3′UTR | 4 | 3838 | agttggtaaggtgcacacag | 54 | 34 | 2 |
| 153085 | Coding | 4 | 1865 | gaaacctggtattgcctttg | 24 | 35 | 2 |
| 153086 | 3′UTR | 4 | 2965 | accagacagtaatagctata | 78 | 36 | 2 |
| 153087 | 5′UTR | 4 | 35 | tagcttgtgaacgcagaagg | 61 | 37 | 2 |
| 153088 | Coding | 4 | 851 | cttgcagtcctcattcgagt | 31 | 38 | 2 |
| 153089 | 3′UTR | 4 | 4289 | ttcactgcacacaggaccag | 58 | 39 | 2 |
| 153090 | 5′UTR | 4 | 38 | acttagcttgtgaacgcaga | 68 | 40 | 2 |
| 153091 | Coding | 4 | 2286 | tagaatccaagagttttgtc | 19 | 41 | 2 |
| 153092 | 3′UTR | 4 | 4020 | agattagtgaataccaatat | 32 | 42 | 2 |
| 153093 | Coding | 4 | 1822 | tgccgccctcctaacatgtt | 66 | 43 | 2 |
| 153094 | Coding | 4 | 275 | tgattgagaagcgacagcca | 65 | 44 | 2 |
| 153095 | 3′UTR | 4 | 2828 | gaaaatttcatccagccaac | 19 | 45 | 2 |
| 153096 | 3′UTR | 4 | 3549 | gtgagaggaattactttgtc | 67 | 46 | 2 |
| 153097 | 3′UTR | 4 | 2635 | cgactcaactgcttctgttg | 7 | 47 | 2 |
| 153098 | 3′UTR | 4 | 3291 | ctataccagttaggactgtt | 90 | 48 | 2 |
| 153099 | 3′UTR | 4 | 3787 | aataattttcaacagtgaag | 19 | 49 | 2 |
| 153100 | Coding | 4 | 1662 | taccaggattttcagaggtt | 76 | 50 | 2 |
| 153101 | 3′UTR | 4 | 3826 | gcacacagaaagggctacta | 66 | 51 | 2 |
| 153102 | Coding | 4 | 1946 | tctccaccccagagcaaatg | 44 | 52 | 2 |
| 153103 | Coding | 4 | 1675 | actattgttttgttaccagg | 66 | 53 | 2 |
| 153104 | Coding | 4 | 2018 | agtcattctctgctcattaa | 51 | 54 | 2 |
| 153105 | Coding | 4 | 2338 | tccaaaaatgtttggaagca | 48 | 55 | 2 |
| 153106 | Coding | 4 | 631 | tggcccttcaaatgttgctg | 1 | 56 | 2 |
| 153107 | 3′UTR | 4 | 2829 | agaaaatttcatccagccaa | 70 | 57 | 2 |
| 153108 | 3′UTR | 4 | 3515 | tcagctgtgttacagctggt | 46 | 58 | 2 |
| 153109 | Coding | 4 | 351 | tggacagatctggctgctgc | 73 | 59 | 2 |
| 153110 | 3′UTR | 4 | 3252 | attctccactgaagcagata | 81 | 60 | 2 |
| 153111 | 3′UTR | 4 | 4253 | cccctagagcaaactgtttg | 70 | 61 | 2 |
| 153112 | 3′UTR | 4 | 4581 | attgctggtacctctatgca | 72 | 62 | 2 |
| 153113 | Start Codon | 4 | 114 | tcagtgaatatcaactctgg | 54 | 63 | 2 |
| 153114 | 3′UTR | 4 | 2716 | cacatattaaggtttctaat | 34 | 64 | 2 |
| 153115 | 3′UTR | 4 | 4142 | atatataacatgtcatgata | 38 | 65 | 2 |
| 153116 | Coding | 4 | 1744 | aacacttcaggttcaataac | 3 | 66 | 2 |

As shown in Table 1, SEQ ID NOs 32, 33, 34, 36, 37, 39, 40, 43, 44, 46, 48, 50, 51, 52, 53, 54, 55, 57, 58, 59, 60, 61, 62, 63 and 65 demonstrated at least 38% inhibition of human glucocorticoid receptor expression in this assay and are therefore preferred. The target regions to which these preferred sequences are complementary are herein referred to as “preferred target segments” and are therefore preferred for targeting by compounds of the present invention. SEQ ID NO: 55 is a cross species oligonucleotide which is also complementary to the mouse glucocorticoid nucleic acid target.

The compounds in Table 2 were analyzed for their effect on human glucocorticoid receptor mRNA levels by quantitative real-time PCR as described in other examples herein. Data, shown in Table 2, are averages from two experiments in which HepG2 cells were treated with 150 nM the antisense oligonucleotides of the present invention. The positive control for each datapoint is identified in the table by sequence ID number. If present, “N.D.” indicates “no data”.

TABLE 2
Inhibition of human glucocorticoid receptor
mRNA levels by chimeric phosphorothioate
oligonucleotides having 2′-MOE wings and a deoxy gap

| | | TARGET | | | | SEQ | CONTROL |
|---|---|---|---|---|---|---|---|
| | | SEQ ID | TARGET | | % | ID | SEQ |
| ISIS # | REGION | NO | SITE | SEQUENCE | INHIB | NO | ID NO |
| 180270 | Coding | 4 | 251 | gggtgaagacgcagaaacct | 47 | 67 | 2 |
| 180271 | Coding | 4 | 388 | tctcccatatacagtcccat | 70 | 68 | 2 |
| 180272 | Coding | 4 | 497 | gtttgcaatgctttcttcca | 45 | 69 | 2 |
| 180273 | Coding | 4 | 507 | acctattgaggtttgcaatg | 69 | 70 | 2 |
| 180274 | Coding | 4 | 514 | ctggtcgacctattgaggtt | 67 | 71 | 2 |
| 180275 | Coding | 4 | 672 | ctgtggtatacaatttcaca | 94 | 72 | 2 |
| 180276 | Coding | 4 | 679 | ctttggtctgtggtatacaa | 90 | 73 | 2 |
| 180277 | Coding | 4 | 687 | caaaggtgctttggtctgtg | 89 | 74 | 2 |
| 180278 | Coding | 4 | 712 | gaaaactccaaatcctgcaa | 64 | 75 | 2 |
| 180279 | Coding | 4 | 877 | ggtttagtgtccggtaaaat | 45 | 76 | 2 |
| 180280 | Coding | 4 | 1000 | ttctcttgcttaattacccc | 69 | 77 | 2 |
| 180281 | Coding | 4 | 1007 | gcccagtttctcttgcttaa | 76 | 78 | 2 |
| 180282 | Coding | 4 | 1072 | gaaatggcagacattttatt | 67 | 79 | 2 |
| 180283 | Coding | 4 | 1081 | ccatgaacagaaatggcaga | 92 | 80 | 2 |
| 180284 | Coding | 4 | 1102 | tgtcctccagaggtactcac | 87 | 81 | 2 |
| 180285 | Coding | 4 | 1112 | gtggtacatctgtcctccag | 56 | 82 | 2 |
| 180286 | Coding | 4 | 1122 | tcatgtcatagtggtacatc | 82 | 83 | 2 |
| 180287 | Coding | 4 | 1132 | gatgctgtattcatgtcata | 21 | 84 | 2 |
| 180288 | Coding | 4 | 1141 | tgagaaagggatgctgtatt | 78 | 85 | 2 |
| 180289 | Coding | 4 | 1181 | tggtggaatgacattaaaaa | 81 | 86 | 2 |
| 180290 | Coding | 4 | 1186 | ggaattggtggaatgacatt | 50 | 87 | 2 |
| 180291 | Coding | 4 | 1387 | gagcacaccaggcagagttt | 47 | 88 | 2 |
| 180292 | Coding | 4 | 1469 | ctgtccttccactgctcttt | 61 | 89 | 2 |
| 180293 | Coding | 4 | 1479 | ggtaattgtgctgtccttcc | 21 | 90 | 2 |
| 180294 | Coding | 4 | 1552 | tttcgatagcggcatgctgg | 78 | 91 | 2 |
| 180295 | Coding | 4 | 1561 | tgaagacattttcgatagcg | 63 | 92 | 2 |
| 180296 | Coding | 4 | 1591 | gtttttcgagcttccaggtt | 55 | 93 | 2 |
| 180297 | Coding | 4 | 1680 | caggaactattgttttgtta | 73 | 94 | 2 |
| 180298 | Coding | 4 | 1852 | gcctttgcccatttcactgc | 53 | 95 | 2 |
| 180300 | Coding | 4 | 2001 | taataatcagatcaggagca | 34 | 96 | 2 |
| 180301 | Coding | 4 | 2008 | tgctcattaataatcagatc | 73 | 97 | 2 |
| 180302 | Coding | 4 | 2015 | cattctctgctcattaataa | 42 | 98 | 2 |
| 180303 | Coding | 4 | 2026 | cagggtagagtcattctctg | 82 | 99 | 2 |
| 180304 | Coding | 4 | 2053 | agcatgtgtttacattggtc | 63 | 100 | 2 |
| 180305 | Coding | 4 | 2110 | atacagagatactcttcata | 59 | 101 | 2 |
| 180306 | Coding | 4 | 2120 | taaggttttcatacagagat | 68 | 102 | 2 |
| 180307 | Coding | 4 | 2131 | gagagaagcagtaaggtttt | 22 | 103 | 2 |
| 180309 | Coding | 4 | 2213 | ggcttttcctagctctttga | 41 | 104 | 2 |
| 180310 | Coding | 4 | 2221 | ttgacaatggcttttcctag | 76 | 105 | 2 |
| 180311 | Coding | 4 | 2386 | gtgatgatttcagctaacat | 57 | 106 | 2 |
| 180315 | 3′UTR | 4 | 2617 | tgccaagtcttggccctcta | 80 | 107 | 2 |
| 180316 | 3′UTR | 4 | 2627 | ctgcttctgttgccaagtct | 75 | 108 | 2 |
| 305186 | 5′UTR | 4 | 13 | caggagggaaatatattttt | 48 | 109 | 2 |
| 305187 | 5′UTR | 4 | 41 | acaacttagcttgtgaacgc | 54 | 110 | 2 |
| 305188 | 5′UTR | 4 | 100 | ctctggcagaggagccgctc | 77 | 111 | 2 |
| 305189 | Start Codon | 4 | 118 | tccatcagtgaatatcaact | 58 | 112 | 2 |
| 305190 | Start Codon | 4 | 125 | tttggagtccatcagtgaat | 72 | 113 | 2 |
| 305191 | Start Codon | 4 | 132 | atgattctttggagtccatc | 76 | 114 | 2 |
| 305192 | Coding | 4 | 205 | ttatagaagtccatcacatc | 55 | 115 | 2 |
| 305193 | Coding | 4 | 243 | acgcagaaaccttcacagta | 67 | 116 | 2 |
| 305194 | Coding | 4 | 358 | actgctttggacagatctgg | 60 | 117 | 2 |
| 305195 | Coding | 4 | 667 | gtatacaatttcacattgcc | 79 | 118 | 2 |
| 305196 | Coding | 4 | 695 | caaaatgtcaaaggtgcttt | 81 | 119 | 2 |
| 305197 | Coding | 4 | 763 | aggtctgatctccaaggact | 77 | 120 | 2 |
| 305198 | Coding | 4 | 826 | tccaaaaggaatgaatcgtc | 85 | 121 | 2 |
| 305199 | Coding | 4 | 1067 | ggcagacattttattaccaa | 68 | 122 | 2 |
| 305200 | Coding | 4 | 1150 | tcctgctgttgagaaaggga | 85 | 123 | 2 |
| 305201 | Coding | 4 | 1224 | cagatccttggcacctattc | 89 | 124 | 2 |
| 305202 | Coding | 4 | 1250 | ccccagagaagtcaagttgt | 0 | 125 | 2 |
| 305203 | Coding | 4 | 1356 | ctgttgttgctgttgaggag | 72 | 126 | 2 |
| 305204 | Coding | 4 | 1737 | caggttcaataacctccaac | 71 | 127 | 2 |
| 305205 | Coding | 4 | 1819 | cgccctcctaacatgttgag | 60 | 128 | 2 |
| 305206 | Coding | 4 | 1870 | ttcctgaaacctggtattgc | 45 | 129 | 2 |
| 305207 | Coding | 4 | 1980 | aacacagcaggtttgcactt | 69 | 130 | 2 |
| 305208 | Coding | 4 | 2146 | tccttaggaactgaagagag | 67 | 131 | 2 |
| 305209 | Coding | 4 | 2175 | catcaaatagctcttggctc | 56 | 132 | 2 |
| 305210 | Coding | 4 | 2201 | ctctttgatgtaggtcattc | 62 | 133 | 2 |
| 305211 | Coding | 4 | 2282 | atccaagagttttgtcagtt | 39 | 134 | 2 |
| 305212 | Coding | 4 | 2304 | tttcaaccacttcatgcata | 71 | 135 | 2 |
| 305213 | Coding | 4 | 2397 | gtatctgattggtgatgatt | 75 | 136 | 2 |
| 305214 | Stop Codon | 4 | 2455 | taaggcagtcacttttgatg | 74 | 137 | 2 |
| 305215 | 3′UTR | 4 | 2488 | taattcgactttctttaagg | 64 | 138 | 2 |
| 305216 | 3′UTR | 4 | 2519 | acaaactgatagtttataca | 41 | 139 | 2 |
| 305217 | 3′UTR | 4 | 2584 | gtgcgtatttaaaacaaaac | 56 | 140 | 2 |
| 305218 | 3′UTR | 4 | 4739 | taatttctccaaaatactga | 53 | 141 | 2 |
| 305219 | 3′UTR | 4 | 2646 | aaaagtgatgacgactcaac | 55 | 142 | 2 |
| 305220 | 3′UTR | 4 | 2723 | ttacgtccacatattaaggt | 67 | 143 | 2 |
| 305221 | 3′UTR | 4 | 2753 | ttaggtgccatccttctttg | 87 | 144 | 2 |
| 305222 | 3′UTR | 4 | 2764 | gcactggtggtttaggtgcc | 77 | 145 | 2 |
| 305223 | 3′UTR | 4 | 2769 | tttgggcactggtggtttag | 82 | 146 | 2 |
| 305224 | 3′UTR | 4 | 2824 | atttcatccagccaactgtg | 82 | 147 | 2 |
| 305225 | 3′UTR | 4 | 2850 | ggatacaccaacagaaagtc | 62 | 148 | 2 |
| 305226 | 3′UTR | 4 | 2939 | acaacttcccttttctgata | 62 | 149 | 2 |
| 305227 | 3′UTR | 4 | 2959 | cagtaatagctataaaaggc | 77 | 150 | 2 |
| 305228 | 3′UTR | 4 | 3004 | agcaagcgtagttcactaaa | 88 | 151 | 2 |
| 305229 | 3′UTR | 4 | 3063 | gctgcccatcttaaacagct | 61 | 152 | 2 |
| 305230 | 3′UTR | 4 | 3132 | aagcaccaacccattttcac | 63 | 153 | 2 |
| 305231 | 3′UTR | 4 | 3144 | ccatcaggttagaagcacca | 72 | 154 | 2 |
| 305232 | 3′UTR | 4 | 3160 | ttctgatagctaagtgccat | 80 | 155 | 2 |
| 305233 | 3′UTR | 4 | 3195 | aagaatactggagatttgag | 75 | 156 | 2 |
| 305234 | 3′UTR | 4 | 3294 | gctctataccagttaggact | 94 | 157 | 2 |
| 305235 | 3′UTR | 4 | 3320 | ttacccagcaggtcactgga | 92 | 158 | 2 |
| 305236 | 3′UTR | 4 | 3330 | catccacagtttacccagca | 86 | 159 | 2 |
| 305237 | 3′UTR | 4 | 3347 | tagtcttttgcaaccatcat | 89 | 160 | 2 |
| 305238 | 3′UTR | 4 | 3375 | agggcctcttggtagttatt | 83 | 161 | 2 |
| 305239 | 3′UTR | 4 | 3409 | tagccattgcaaaaataggg | 71 | 162 | 2 |
| 305240 | 3′UTR | 4 | 3416 | tgccatatagccattgcaaa | 89 | 163 | 2 |
| 305241 | 3′UTR | 4 | 3445 | ctgaaagacaaatagtttac | 29 | 164 | 2 |
| 305242 | 3′UTR | 4 | 3484 | acaacttttaagaagttata | 32 | 165 | 2 |
| 305243 | 3′UTR | 4 | 3499 | tggttatctggaatcacaac | 82 | 166 | 2 |
| 305244 | 3′UTR | 4 | 3504 | acagctggttatctggaatc | 81 | 167 | 2 |
| 305245 | 3′UTR | 4 | 3521 | agtctctcagctgtgttaca | 81 | 168 | 2 |
| 305246 | 3′UTR | 4 | 3610 | gtgaaaatgggtgtctagcc | 51 | 169 | 2 |
| 305247 | 3′UTR | 4 | 3624 | tgacagatgggaatgtgaaa | 67 | 170 | 2 |
| 305248 | 3′UTR | 4 | 3641 | aaagattaaccaattggtga | 80 | 171 | 2 |
| 305249 | 3′UTR | 4 | 3658 | tttcctgtaccatcaggaaa | 83 | 172 | 2 |
| 305250 | 3′UTR | 4 | 3743 | tctatggcacacattaggga | 67 | 173 | 2 |
| 305251 | 3′UTR | 4 | 3754 | ttgtgttaaactctatggca | 74 | 174 | 2 |
| 305252 | 3′UTR | 4 | 3770 | aagaaattcacaggacttgt | 76 | 175 | 2 |
| 305253 | 3′UTR | 4 | 3841 | gaaagttggtaaggtgcaca | 84 | 176 | 2 |
| 305254 | 3′UTR | 4 | 3886 | caaatttcttgtggcttagt | 69 | 177 | 2 |
| 305255 | 3′UTR | 4 | 3898 | ttgaatagaaatcaaatttc | 0 | 178 | 2 |
| 305256 | 3′UTR | 4 | 3918 | acacaaataatttggccacc | 58 | 179 | 2 |
| 305257 | 3′UTR | 4 | 3923 | ctattacacaaataatttgg | 22 | 180 | 2 |
| 305258 | 3′UTR | 4 | 4038 | agtagcccttcccttcccag | 33 | 181 | 2 |
| 305259 | 3′UTR | 4 | 4046 | aaagctgcagtagcccttcc | 56 | 182 | 2 |
| 305260 | 3′UTR | 4 | 4053 | tgcatgtaaagctgcagtag | 77 | 183 | 2 |
| 305261 | 3′UTR | 4 | 4065 | attttaataaattgcatgta | 24 | 184 | 2 |
| 305262 | 3′UTR | 4 | 4082 | caagctattttacaatcatt | 57 | 185 | 2 |
| 305263 | 3′UTR | 4 | 4174 | atccatcagcatttctttga | 74 | 186 | 2 |
| 305264 | 3′UTR | 4 | 4191 | tataaatcatataggttatc | 26 | 187 | 2 |
| 305265 | 3′UTR | 4 | 4244 | caaactgtttggtttctgag | 77 | 188 | 2 |
| 305266 | 3′UTR | 4 | 4311 | ctgggtcagagcctcagcaa | 84 | 189 | 2 |
| 305267 | 3′UTR | 4 | 4319 | taatctcactgggtcagagc | 79 | 190 | 2 |
| 305268 | 3′UTR | 4 | 4365 | aatgagaagggtggtcagaa | 77 | 191 | 2 |
| 305269 | 3′UTR | 4 | 4376 | ctcactgttggaatgagaag | 82 | 192 | 2 |
| 305270 | 3′UTR | 4 | 4401 | agtaaactaaacctgcgctg | 74 | 193 | 2 |
| 305271 | 3′UTR | 4 | 4442 | ctgtttacatactttacata | 68 | 194 | 2 |
| 305272 | 3′UTR | 4 | 4483 | agatggtgcctttaaggatg | 70 | 195 | 2 |
| 305273 | 3′UTR | 4 | 4504 | atgtgaaagtaacccgctat | 74 | 196 | 2 |
| 305274 | 3′UTR | 4 | 4547 | ttctgaagcttctgttgtca | 93 | 197 | 2 |
| 305275 | 3′UTR | 4 | 4577 | ctggtacctctatgcaaact | 90 | 198 | 2 |
| 305276 | 3′UTR | 4 | 4602 | gagattctgcactatttaca | 81 | 199 | 2 |
| 305277 | 3′UTR | 4 | 4624 | tagtgtattattggcaacct | 79 | 200 | 2 |
| 305278 | 3′UTR | 4 | 4664 | ttatttggaaataaactctt | 27 | 201 | 2 |
| 305279 | 3′UTR | 4 | 4680 | aaaacatgtcctcattttat | 62 | 202 | 2 |
| 305280 | Intron | 25 | 103636 | gaagctctttttgaaactta | 83 | 203 | 2 |
| 305281 | Coding | 26 | 2315 | tggttttaaccacataacat | 79 | 204 | 2 |
| 305282 | Coding | 26 | 2304 | acataacattttcatgcata | 33 | 205 | 2 |
| 305283 | 3′UTR | 25 | 104039 | tttgttgtgagtaaccaact | 78 | 206 | 2 |
| 305284 | 3′UTR | 25 | 104061 | acactaaaaatacttttcag | 24 | 207 | 2 |
| 305285 | 3′UTR | 25 | 104562 | aactccacccaaagggttta | 71 | 208 | 2 |
| 305286 | 3′UTR | 25 | 104629 | ttcctgaaaacctggtcact | 54 | 209 | 2 |
| 305287 | Intron | 25 | 24125 | attaatctgcataggaagca | 73 | 210 | 2 |
| 305288 | Exon 7: | 25 | 87671 | ttctaccaacctgaagagag | 54 | 211 | 2 |
| | intron 8 | | | | | | |
| | junction | | | | | | |
| 305289 | Intron | 25 | 89336 | agaagaactcgtgatattat | 77 | 212 | 2 |
| 305290 | Intron 7: | 25 | 100360 | tccttaggaactaaaaggtt | 43 | 213 | 2 |
| | exon8 | | | | | | |
| | junction | | | | | | |
| 305291 | Intron 8: | 25 | 101044 | tttcaaccacctgcaagaga | 68 | 214 | 2 |
| | exon 9 | | | | | | |
| | junction | | | | | | |
| 305292 | Intron | 27 | 196 | ggtcccagctgcttcggccg | 36 | 215 | 2 |
| 305293 | Intron | 27 | 304 | ggagagcccctatttaagaa | 47 | 216 | 2 |

As shown in Table 2, SEQ ID NOs 67, 68, 69, 70, 71, 72, 73, 74, 75, 76, 77, 78, 79, 80, 81, 82, 83, 85, 86, 87, 88, 89, 91, 92, 93, 94, 95, 97, 98, 99, 100, 101, 102, 104, 105, 106, 107, 108, 109, 110, 111, 112, 113, 114, 115, 116, 117, 118, 119, 120, 121, 122, 123, 124, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 166, 167, 168, 169, 170, 171, 172, 173, 174, 175, 176, 177, 179, 182, 183, 185, 186, 188, 189, 190, 191, 192, 193, 194, 195, 196, 197, 198, 199, 200, 202, 203, 204, 206, 208, 209, 210, 211, 212, 213, 214 and 216 demonstrated at least 39% inhibition of human glucocorticoid receptor expression in this assay and are therefore preferred. The target regions to which these preferred sequences are complementary are herein referred to as “preferred target segments” and are therefore preferred for targeting by compounds of the present invention.

SEQ ID NOs 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107 and 108 are cross species oligonucleotides which are also complementary to the mouse glucocorticoid receptor nucleic acid target.

### Example 12

### Antisense Inhibition of Mouse Glucocorticoid Receptor Expression by Chimeric Phosphorothioate Oligonucleotides Having 2′-MOE Wings and a Deoxy Gap

In accordance with the present invention, a second series of antisense compounds was designed to target different regions of the mouse glucocorticoid receptor RNA, using published sequences (GenBank accession number NM_{—}008173.1, incorporated herein as SEQ ID NO: 11, GenBank accession number X66367.1, incorporated herein as SEQ ID NO: 217, GenBank accession number BF181849.1, incorporated herein as SEQ ID NO: 218, GenBank accession number BE373661.1, incorporated herein as SEQ ID NO: 219, and the complement of nucleotides 145001 to 164000 of the sequence with GenBank accession number AC007995.19, incorporated herein as SEQ ID NO: 220). The compounds are shown in Table 3. “Target site” indicates the first (5′-most) nucleotide number on the particular target nucleic acid to which the compound binds. All compounds in Table 3 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytosine residues are 5-methylcytosines. The compounds were analyzed for their effect on mouse glucocorticoid receptor mRNA levels by quantitative real-time PCR as described in other examples herein. Data are averages from two experiments in which b.END cells were treated with 150 nM of the antisense oligonucleotides of the present invention. The positive control for each datapoint is identified in the table by sequence ID number. If present, “N.D.” indicates “no data”.

TABLE 3
Inhibition of mouse glucocorticoid receptor
mRNA levels by chimeric phosphorothioate
oligonucleotides having 2′-MOE wings and a deoxy gap

| | | TARGET | | | | SEQ | CONTROL |
|---|---|---|---|---|---|---|---|
| | | SEQ ID | TARGET | | % | ID | SEQ |
| ISIS # | REGION | NO | SITE | SEQUENCE | INHIB | NO | ID NO |
| 153105 | Coding | 11 | 2242 | tccaaaaatgtttggaagca | 64 | 55 | 2 |
| 180268 | Start Codon | 11 | 2 | cattggcaaatattaacttc | 53 | 221 | 2 |
| 180269 | Start Codon | 11 | 11 | tttggagtccattggcaaat | 70 | 222 | 2 |
| 180270 | Coding | 11 | 140 | gggtgaagacgcagaaacct | 58 | 67 | 2 |
| 180271 | Coding | 11 | 301 | tctcccatatacagtcccat | 86 | 68 | 2 |
| 180272 | Coding | 11 | 410 | gtttgcaatgctttcttcca | 86 | 69 | 2 |
| 180273 | Coding | 11 | 420 | acctattgaggtttgcaatg | 65 | 70 | 2 |
| 180274 | Coding | 11 | 427 | ctggtcgacctattgaggtt | 69 | 71 | 2 |
| 180275 | Coding | 11 | 585 | ctgtggtatacaatttcaca | 84 | 72 | 2 |
| 180276 | Coding | 11 | 592 | ctttggtctgtggtatacaa | 84 | 73 | 2 |
| 180277 | Coding | 11 | 600 | caaaggtgctttggtctgtg | 82 | 74 | 2 |
| 180278 | Coding | 11 | 625 | gaaaactccaaatcctgcaa | 93 | 75 | 2 |
| 180279 | Coding | 11 | 787 | ggtttagtgtccggtaaaat | 75 | 76 | 2 |
| 180280 | Coding | 11 | 910 | ttctcttgcttaattacccc | 87 | 77 | 2 |
| 180281 | Coding | 11 | 917 | gcccagtttctcttgcttaa | 84 | 78 | 2 |
| 180282 | Coding | 11 | 982 | gaaatggcagacattttatt | 72 | 79 | 2 |
| 180283 | Coding | 11 | 991 | ccatgaacagaaatggcaga | 74 | 80 | 2 |
| 180284 | Coding | 11 | 1012 | tgtcctccagaggtactcac | 82 | 81 | 2 |
| 180285 | Coding | 11 | 1022 | gtggtacatctgtcctccag | 66 | 82 | 2 |
| 180286 | Coding | 11 | 1032 | tcatgtcatagtggtacatc | 69 | 83 | 2 |
| 180287 | Coding | 11 | 1042 | gatgctgtattcatgtcata | 70 | 84 | 2 |
| 180288 | Coding | 11 | 1051 | tgagaaagggatgctgtatt | 62 | 85 | 2 |
| 180289 | Coding | 11 | 1091 | tggtggaatgacattaaaaa | 71 | 86 | 2 |
| 180290 | Coding | 11 | 1096 | ggaattggtggaatgacatt | 63 | 87 | 2 |
| 180291 | Coding | 11 | 1294 | gagcacaccaggcagagttt | 54 | 88 | 2 |
| 180292 | Coding | 11 | 1376 | ctgtccttccactgctcttt | 63 | 89 | 2 |
| 180293 | Coding | 11 | 1386 | ggtaattgtgctgtccttcc | 57 | 90 | 2 |
| 180294 | Coding | 11 | 1459 | tttcgatagcggcatgctgg | 55 | 91 | 2 |
| 180295 | Coding | 11 | 1468 | tgaagacattttcgatagcg | 57 | 92 | 2 |
| 180296 | Coding | 11 | 1498 | gtttttcgagcttccaggtt | 59 | 93 | 2 |
| 180297 | Coding | 11 | 1584 | caggaactattgttttgtta | 41 | 94 | 2 |
| 180298 | Coding | 11 | 1756 | gcctttgcccatttcactgc | 41 | 95 | 2 |
| 180299 | Coding | 11 | 1774 | tttctgaatcctggtatcgc | 48 | 223 | 2 |
| 180300 | Coding | 11 | 1905 | taataatcagatcaggagca | 43 | 96 | 2 |
| 180301 | Coding | 11 | 1912 | tgctcattaataatcagatc | 62 | 97 | 2 |
| 180302 | Coding | 11 | 1919 | cattctctgctcattaataa | 50 | 98 | 2 |
| 180303 | Coding | 11 | 1930 | cagggtagagtcattctctg | 63 | 99 | 2 |
| 180304 | Coding | 11 | 1957 | agcatgtgtttacattggtc | 71 | 100 | 2 |
| 180305 | Coding | 11 | 2014 | atacagagatactcttcata | 49 | 101 | 2 |
| 180306 | Coding | 11 | 2024 | taaggttttcatacagagat | 47 | 102 | 2 |
| 180307 | Coding | 11 | 2035 | gagagaagcagtaaggtttt | 26 | 103 | 2 |
| 180308 | Coding | 11 | 2050 | tccttaggaactgaggagag | 46 | 224 | 2 |
| 180309 | Coding | 11 | 2117 | ggcttttcctagctctttga | 69 | 104 | 2 |
| 180310 | Coding | 11 | 2125 | ttgacaatggcttttcctag | 65 | 105 | 2 |
| 180311 | Coding | 11 | 2290 | gtgatgatttcagctaacat | 59 | 106 | 2 |
| 180312 | Stop Codon | 11 | 2359 | taaggcagtcatttctgatg | 58 | 225 | 2 |
| 180313 | 3′UTR | 11 | 2376 | aaggcagcctttcttagtaa | 51 | 226 | 2 |
| 180314 | 3′UTR | 11 | 2414 | aagtttgtacagtaaaagct | 85 | 227 | 2 |
| 180315 | 3′UTR | 11 | 2511 | tgccaagtcttggccctcta | 13 | 107 | 2 |
| 180316 | 3′UTR | 11 | 2521 | ctgcttctgttgccaagtct | 52 | 108 | 2 |
| 180317 | 3′UTR | 11 | 2527 | gctcatctgcttctgttgcc | 68 | 228 | 2 |
| 180318 | 5′UTR | 217 | 1386 | gcatacatactgtgagcccg | 0 | 229 | 2 |
| 180319 | 5′UTR | 218 | 37 | ctgggcggccccgtctgcag | 14 | 230 | 2 |
| 180320 | 5′UTR | 218 | 104 | ttggcaaatattaatgtgag | 20 | 231 | 2 |
| 180321 | 5′UTR | 219 | 227 | agccagataaacaagtcggc | 64 | 232 | 2 |
| 180322 | 5′UTR | 219 | 278 | atattaactcagcaccggcg | 37 | 233 | 2 |
| 180323 | Intron | 220 | 4092 | agaatcttagctatagggct | 35 | 234 | 2 |
| 180324 | Exon 5: | 220 | 7968 | catgccttacctggtatcgc | 8 | 235 | 2 |
| | Intron 5 | | | | | | |
| | junction | | | | | | |
| 180325 | Exon 6: | 220 | 9049 | tgtaacttactcattaataa | 32 | 236 | 2 |
| | Intron 6 | | | | | | |
| | junction | | | | | | |
| 180326 | intron | 220 | 13238 | tcacatagtctgcgattgtt | 63 | 237 | 2 |
| 180327 | Intron 7: | 220 | 14602 | tccttaggaactaaaaggta | 13 | 238 | 2 |
| | Exon 8 | | | | | | |
| | junction | | | | | | |
| 180328 | 3′ UTR | 220 | 14909 | tatctctgactgtcctggca | 59 | 239 | 2 |
| 180329 | 3′ UTR | 220 | 15984 | agcctttcttagtaaggcag | 36 | 240 | 2 |
| 180330 | 3′ UTR | 220 | 16177 | acatcactgtctgctttcct | 59 | 241 | 2 |
| 180331 | 3′ UTR | 220 | 16227 | ggacatgtctccactaactg | 65 | 242 | 2 |
| 180332 | 3′ UTR | 220 | 16268 | tttgggcactggtggttcag | 63 | 243 | 2 |
| 180333 | 3′ UTR | 220 | 16548 | catcttaaacagctatacaa | 64 | 244 | 2 |
| 180334 | 3′ UTR | 220 | 16639 | ctgatagctgagtgccatca | 55 | 245 | 2 |
| 180335 | 3′ UTR | 220 | 16868 | agagatggtgcattgggtgc | 48 | 246 | 2 |
| 180336 | 3′ UTR | 220 | 17166 | cctgacattcagttctaaat | 70 | 247 | 2 |
| 180337 | 3′ UTR | 220 | 17178 | caaacatggatgcctgacat | 70 | 248 | 2 |
| 180338 | 3′ UTR | 220 | 17215 | ttagattctatggcacatgt | 52 | 249 | 2 |
| 180339 | 3′ UTR | 220 | 17327 | gttaagctttgagtcacaga | 64 | 250 | 2 |
| 180340 | 3′ UTR | 220 | 17729 | ctctccctagcttagagcaa | 71 | 251 | 2 |
| 180341 | 3′ UTR | 220 | 17909 | tggacggtgcctctaagtac | 65 | 252 | 2 |
| 180342 | 3′ UTR | 220 | 18076 | gaaatggactcttgtaggat | 65 | 253 | 2 |
| 180343 | 3′ UTR | 220 | 18283 | ataaatttcacatccagctg | 62 | 254 | 2 |
| 180344 | 3′ UTR | 220 | 18370 | taaatgtacaataatctatt | 14 | 255 | 2 |

As shown in Table 3, SEQ ID NOs 55, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 104, 105, 106, 108, 221, 222, 223, 224, 225, 226, 227, 228, 232, 233, 234, 236, 237, 239, 240, 241, 242, 243, 244, 245, 246, 247, 248, 249, 250, 251, 252, 253 and 254 demonstrated at least 32% inhibition of mouse glucocorticoid receptor expression in this experiment and are therefore preferred. The target regions to which these preferred sequences are complementary are herein referred to as “preferred target segments” and are therefore preferred for targeting by compounds of the present invention.

SEQ ID NOs, 69, 70, 71, 74, 76, 77, 78, 79, 80, 81, 82, 83, 84, 85, 86, 87, 89, 90, 94, 95, 96, 97, 98, 100, 101, 102, 103, 104, 106, 222, 224, 227, 231, 254 and 255 are cross species oligonucleotides which are also complementary to the rat glucocorticoid receptor nucleic acid target.

### Example 13

### Antisense Inhibition of Rat Glucocorticoid Receptor Expression by Chimeric Phosphorothioate Oligonucleotides Having 2′-MOE Wings and a Deoxy Gap

In accordance with the present invention, a third series of antisense compounds was designed to target different regions of the rat glucocorticoid receptor RNA, using published sequences (GenBank accession number NM_{—}012576.1, incorporated herein as SEQ ID NO: 18, and GenBank accession number Y00489.1, incorporated herein as SEQ ID NO: 256). The compounds are shown in Table 4. “Target site” indicates the first (5′-most) nucleotide number on the particular target nucleic acid to which the compound binds. All compounds in Table 4 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytosine residues are 5-methylcytosines. The compounds were analyzed for their effect on rat glucocorticoid receptor mRNA levels by quantitative real-time PCR as described in other examples herein. Data are averages from two experiments in which NRK cells were treated with 150 nM of the antisense oligonucleotides of the present invention. The positive control for each datapoint is identified in the table by sequence ID number. If present, “N.D.” indicates “no data”.

TABLE 4
Inhibition of rat glucocorticoid receptor
mRNA levels by chimeric phosphorothioate oligonucleotides
having 2′-MOE wings and a deoxy gap

| | | TARGET | | | | SEQ | CONTROL |
|---|---|---|---|---|---|---|---|
| | | SEQ ID | TARGET | | % | ID | SEQ |
| ISIS # | REGION | NO | SITE | SEQUENCE | INHIB | NO | ID NO |
| 180269 | Start | 18 | 61 | tttggagtccattggcaaat | 42 | 222 | 2 |
| | Codon | | | | | | |
| 180272 | Coding | 18 | 496 | gtttgcaatgctttcttcca | 60 | 69 | 2 |
| 180273 | Coding | 18 | 506 | acctattgaggtttgcaatg | 59 | 70 | 2 |
| 180274 | Coding | 18 | 513 | ctggtcgacctattgaggtt | 49 | 71 | 2 |
| 180277 | Coding | 18 | 686 | caaaggtgctttggtctgtg | 68 | 74 | 2 |
| 180279 | Coding | 18 | 873 | ggtttagtgtccggtaaaat | 58 | 76 | 2 |
| 180280 | Coding | 18 | 996 | ttctcttgcttaattacccc | 56 | 77 | 2 |
| 180281 | Coding | 18 | 1003 | gcccagtttctcttgcttaa | 74 | 78 | 2 |
| 180282 | Coding | 18 | 1068 | gaaatggcagacattttatt | 28 | 79 | 2 |
| 180283 | Coding | 18 | 1077 | ccatgaacagaaatggcaga | 52 | 80 | 2 |
| 180284 | Coding | 18 | 1098 | tgtcctccagaggtactcac | 54 | 81 | 2 |
| 180285 | Coding | 18 | 1108 | gtggtacatctgtcctccag | 58 | 82 | 2 |
| 180286 | Coding | 18 | 1118 | tcatgtcatagtggtacatc | 46 | 83 | 2 |
| 180287 | Coding | 18 | 1128 | gatgctgtattcatgtcata | 27 | 84 | 2 |
| 180288 | Coding | 18 | 1137 | tgagaaagggatgctgtatt | 64 | 85 | 2 |
| 180289 | Coding | 18 | 1177 | tggtggaatgacattaaaaa | 47 | 86 | 2 |
| 180290 | Coding | 18 | 1182 | ggaattggtggaatgacatt | 53 | 87 | 2 |
| 180292 | Coding | 18 | 1462 | ctgtccttccactgctcttt | 37 | 89 | 2 |
| 180293 | Coding | 18 | 1472 | ggtaattgtgctgtccttcc | 30 | 90 | 2 |
| 180297 | Coding | 18 | 1670 | caggaactattgttttgtta | 56 | 94 | 2 |
| 180298 | Coding | 18 | 1842 | gcctttgcccatttcactgc | 44 | 95 | 2 |
| 180300 | Coding | 18 | 1991 | taataatcagatcaggagca | 26 | 96 | 2 |
| 180301 | Coding | 18 | 1998 | tgctcattaataatcagatc | 38 | 97 | 2 |
| 180302 | Coding | 18 | 2005 | cattctctgctcattaataa | 10 | 98 | 2 |
| 180304 | Coding | 18 | 2043 | agcatgtgtttacattggtc | 57 | 100 | 2 |
| 180305 | Coding | 18 | 2100 | atacagagatactcttcata | 31 | 101 | 2 |
| 180306 | Coding | 18 | 2110 | taaggttttcatacagagat | 47 | 102 | 2 |
| 180307 | Coding | 18 | 2121 | gagagaagcagtaaggtttt | 16 | 103 | 2 |
| 180308 | Coding | 18 | 2136 | tccttaggaactgaggagag | 58 | 224 | 2 |
| 180309 | Coding | 18 | 2203 | ggcttttcctagctctttga | 54 | 104 | 2 |
| 180311 | Coding | 18 | 2376 | gtgatgatttcagctaacat | 41 | 106 | 2 |
| 180314 | 3′UTR | 18 | 2500 | aagtttgtacagtaaaagct | 39 | 227 | 2 |
| 180320 | 5′UTR | 18 | 50 | ttggcaaatattaatgtgag | 3 | 231 | 2 |
| 180343 | 3′UTR | 18 | 4773 | ataaatttcacatccagctg | 48 | 254 | 2 |
| 180344 | 3′UTR | 18 | 4859 | taaatgtacaataatctatt | 0 | 255 | 2 |
| 223308 | Coding | 256 | 278 | taagtctggctgctgctgct | 41 | 257 | 2 |
| 223309 | Coding | 256 | 285 | ctttggataagtctggctgc | 48 | 258 | 2 |
| 223310 | Coding | 18 | 150 | aggcttttataaaagtccat | 48 | 259 | 2 |
| 223311 | Coding | 18 | 244 | atcaaggagaatcctctgct | 49 | 260 | 2 |
| 223312 | Coding | 18 | 1248 | gcccccaaggaagtcaggct | 48 | 261 | 2 |
| 223313 | Coding | 18 | 1407 | ccgtaatgacatcctgaagc | 41 | 262 | 2 |
| 223314 | Coding | 18 | 2156 | actcttggctcttcagacct | 44 | 263 | 2 |
| 223315 | Stop | 18 | 2445 | taaggcagtcatttttgatg | 53 | 264 | 2 |
| | Codon | | | | | | |
| 223316 | 3′UTR | 18 | 2472 | aactttctttaaggcaacct | 44 | 265 | 2 |
| 223317 | 3′UTR | 18 | 2586 | cctctataaaccacatgtac | 52 | 266 | 2 |
| 223318 | 3′UTR | 18 | 2637 | tgtcatcacttcagagtgtt | 30 | 267 | 2 |
| 223319 | 3′UTR | 18 | 2685 | aactgttagtttctgtgata | 52 | 268 | 2 |
| 223320 | 3′UTR | 18 | 2799 | tagaaagttttacccagcca | 58 | 269 | 2 |
| 223321 | 3′UTR | 18 | 3202 | ctatgtaattctccatggaa | 50 | 270 | 2 |
| 223322 | 3′UTR | 18 | 3266 | ctggactaggtgctctacac | 44 | 271 | 2 |
| 223323 | 3′UTR | 18 | 3366 | attgaagagatggtgcatta | 55 | 272 | 2 |
| 223324 | 3′UTR | 18 | 3473 | ggctttatcagagctggcta | 62 | 273 | 2 |
| 223325 | 3′UTR | 18 | 3542 | ctgtattagcgatttagttg | 53 | 274 | 2 |
| 223326 | 3′UTR | 18 | 3604 | accatgagagctagaccaat | 35 | 275 | 2 |
| 223327 | 3′UTR | 18 | 3686 | gcacatgtagggatgtgtag | 46 | 276 | 2 |
| 223328 | 3′UTR | 18 | 3880 | agtttttctattacacaaat | 21 | 277 | 2 |
| 223329 | 3′UTR | 18 | 3992 | cagtagccctttccctttcc | 11 | 278 | 2 |
| 223330 | 3′UTR | 18 | 4117 | catcaatatttctttgaccc | 23 | 279 | 2 |
| 223331 | 3′UTR | 18 | 4576 | aatggactattgaagggtgg | 44 | 280 | 2 |
| 223332 | 3′UTR | 18 | 4609 | agaaaacataagcatgtcct | 33 | 281 | 2 |
| 223333 | 3′UTR | 18 | 4702 | gaacaatcccttttagagag | 49 | 282 | 2 |
| 223334 | 3′UTR | 18 | 4848 | taatctatttttgagaagct | 52 | 283 | 2 |
| 223335 | 3′UTR | 18 | 5039 | tacgcttcaaggaaagcttc | 59 | 284 | 2 |
| 223336 | 3′UTR | 18 | 5183 | ccgagtctcactgaagttat | 55 | 285 | 2 |
| 223337 | 3′UTR | 18 | 5220 | tctttcaagatcggtcatga | 36 | 286 | 2 |
| 223338 | 3′UTR | 18 | 5274 | ccaaggcctaaaataaccag | 44 | 287 | 2 |
| 223339 | 3′UTR | 18 | 5390 | ctttgggtactctcacttat | 15 | 288 | 2 |
| 223340 | 3′UTR | 18 | 5430 | cctgactcatccttagaccc | 23 | 289 | 2 |
| 223341 | 3′UTR | 18 | 5606 | tctcaagctccatgatcctt | 4 | 290 | 2 |
| 223342 | 3′UTR | 18 | 5680 | cgccttctaacactgaaacc | 27 | 291 | 2 |
| 223343 | 3′UTR | 18 | 5686 | ctgtttcgccttctaacact | 45 | 292 | 2 |
| 223344 | 3′UTR | 18 | 5740 | gtttgggaatgagaagactt | 44 | 293 | 2 |
| 223345 | 3′UTR | 18 | 5785 | tagcagctggtcaccagtcc | 27 | 294 | 2 |
| 223346 | 3′UTR | 18 | 5858 | attttcatacagccatttat | 38 | 295 | 2 |
| 223347 | 3′UTR | 18 | 5908 | tattgacacactgaaatctc | 16 | 296 | 2 |
| 223348 | 3′UTR | 18 | 6119 | tagaaagacggatttttaaa | 0 | 297 | 2 |
| 223349 | 3′UTR | 18 | 6214 | tgtggtttggtaataccaag | 56 | 298 | 2 |
| 223350 | 3′UTR | 18 | 6244 | actaacatttactgccaatt | 28 | 299 | 2 |

As shown in Table 4, SEQ ID NOs 69, 70, 71, 74, 76, 77, 78, 79, 80, 81, 82, 83, 84, 85, 86, 87, 89, 90, 94, 95, 96, 97, 100, 101, 102, 104, 106, 222, 224, 227, 254, 257, 258, 259, 260, 261, 262, 263, 264, 265, 266, 267, 268, 269, 270, 271, 272, 273, 274, 275, 276, 280, 281, 282, 283, 284, 285, 286, 287, 291, 292, 293, 294, 295, 298 and 299 demonstrated at least 26% inhibition of rat glucocorticoid receptor expression in this experiment and are therefore preferred. As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of glucocorticoid receptor.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 14

### Western Blot Analysis of Glucocorticoid Receptor Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to glucocorticoid receptor is used, with a radiolabeled or fluorescently labeled secondary antibody directed against the primary antibody species. Bands are visualized using a PHOSPHORIMAGERT™ (Molecular Dynamics, Sunnyvale Calif.).

### Example 15

### Antisense Inhibition of Mouse Glucocorticoid Receptor: Dose Response Study in b.END Cells

In a further embodiment of the invention, ISIS 180271, ISIS 180272, ISIS 180277, ISIS 180280, ISIS 180281 and ISIS 180314 were tested in a dose response experiment. ISIS 118920 (GTTCATTCTAAAGTGGTCAC, SEQ ID NO: 300) targets protein phosphatase catalytic subunit 2α and was used as a control. b.END cells were plated in 24-well plates at a density of 40,000 cells per well. Cells were then treated with 1, 5, 10, 25, 50, 100 or 200 nM of antisense oligonucleotide, mixed with 3 μl of LIPOFECTIN (Invitrogen Life Technologies, Carlsbad, Calif.) per 100 nM oligonucleotide per 1 ml of media, as described by other examples herein. Expression of mouse glucocorticoid receptor was measured by real-time PCR as described by other examples herein. Data are expressed as percent inhibition of mouse glucocorticoid receptor mRNA, normalized to untreated control cells. The results are the average of three experiments and are shown in Table 5. A “+” preceding the numbers in the control oligonucleotide treated results indicates that gene expression increased.

TABLE 5
Antisense inhibition of mouse glucocorticoid receptor:
dose response in b.END cells

| | | % Inhibition of mouse glucocorticoid receptor | | | | | | |
|---|---|---|---|---|---|---|---|---|
| | | Dose of oligonucleotide | | | | | | |
| | SEQ ID | | | | 25 | 50 | | 200 |
| ISIS # | NO | 1 nM | 5 nM | 10 nM | nM | nM | 100 nM | nM |
| 180271 | 68 | 11 | 39 | 50 | 71 | 79 | 80 | 81 |
| 180272 | 69 | 7 | 33 | 50 | 71 | 77 | 80 | 78 |
| 180277 | 74 | 17 | 51 | 56 | 78 | 88 | 82 | 71 |
| 180280 | 77 | 12 | 60 | 64 | 84 | 86 | 85 | 86 |
| 180281 | 78 | 23 | 72 | 73 | 82 | 89 | 83 | 84 |
| 180314 | 227 | 12 | 24 | 36 | 67 | 74 | 77 | 79 |
| 118920 | 300 | 2 | 1 | +36 | +11 | +20 | +15 | 4 |

As demonstrated in Table 5, the antisense compounds tested in this experiment inhibited mouse glucocorticoid receptor mRNA expression in b.END cells in a dose-dependent manner.

### Example 16

### Antisense Inhibition of Mouse Glucocorticoid Receptor: Dose Response Study in Primary Mouse Hepatocytes

In accordance with the present invention, ISIS 180271, ISIS 180272 and ISIS 180280 were tested in a dose-response experiment in primary mouse hepatocytes, which were treated with 50, 100, 200 or 400 nM of antisense oligonucleotide. ISIS 129685 (AATATTCGCACCCCACTGGT, SEQ ID NO: 301), ISIS 129686 (CGTTATTAACCTCCGTTGAA, SEQ ID NO: 302) and ISIS 129695 (TTCTACCTCGCGCGATTTAC, SEQ ID NO: 303) are oligonucleotides that target protein phosphatase 2A and were used as control oligonucleotides. Cells were treated and mRNA expression levels were measured as described by other examples herein. The data are normalized to untreated control cells and are expressed as percent change in mouse glucocorticoid receptor, where a “−” indicates a decrease in expression and a “+” indicates an increase in expression. The results are the average of 3 experiments and are shown in Table 6.

TABLE 6
Antisense inhibition of mouse glucocorticoid receptor: dose response experiment in primary
mouse hepatocytes

| | | % Change in mouse glucocorticoid | | | |
|---|---|---|---|---|---|
| | | receptor expression | | | |
| | SEQ ID | Dose of oligonucleotide | | | |
| ISIS # | NO | 50 nM | 100 nM | 200 nM | 400 nM |
| 180271 | 68 | −42 | −68 | −77 | −86 |
| 180272 | 69 | −49 | −66 | −73 | −84 |
| 180280 | 77 | −55 | −64 | −74 | −84 |
| 129685 | 301 | +6 | +12 | +3 | −31 |
| 129686 | 302 | +14 | +12 | +5 | +1 |
| 129695 | 303 | −6 | −19 | −10 | −21 |

These data demonstrate that in primary mouse hepatocytes, ISIS 180271, ISIS 180272 and ISIS 180280, unlike the control oligonucleotides, inhibited mouse glucocorticoid receptor expression in a dose-dependent manner.

### Example 17

### Effect of Antisense Inhibitors of Glucocorticoid Receptor on Lean Mice (Db/Db+/−Mice)

db/db+/− mice are heterozygous littermates of db/db mice, often referred to as lean littermates because they do not display the db (obesity and hyperglycemia) phenotype. Six-week old db/db+/− male mice were dosed twice weekly with 50 mg/kg of antisense oligonucleotide, given subcutaneously. A total of five doses were given. Glucocorticoid antisense oligonucleotides used were ISIS 180272 (SEQ ID NO: 69) and ISIS 180280 (SEQ ID NO: 77). ISIS 116847 (CTGCTAGCCTCTGGATTTGA; SEQ ID NO: 304), targeted to mouse PTEN, was used as a positive control. Antisense compounds were prepared in buffered saline and sterilized by filtering through a 0.2 micron filter. Blood samples were obtained from mice and rats via tail snip. Plasma glucose levels were measured before the initial dose (day −6) and the day before the mice were sacrificed (day 15). After sacrifice, serum lipids were measured and target reduction in liver was also measured.

In lean mice treated with ISIS 180272 (SEQ ID NO: 69), an antisense inhibitor of glucocorticoid receptor, plasma glucose levels were approximately 220 mg/dL at day −6 and 170 mg/dL at day 15. In lean mice treated with ISIS 180280 (SEQ ID NO: 77), another antisense inhibitor of glucocorticoid receptor, plasma glucose levels were approximately 230 mg/dL at day −6 and 160 mg/dL at day 15. Lean mice treated with saline alone had fed plasma glucose levels of approximately 240 mg/dL at day −6 and 180 mg/dL at day 15. While plasma glucose levels decreased slightly, the mice did not become hypoglycemic.

Serum lipids were also measured at the end of the study. Cholesterol levels were approximately 90 mg/dL for saline treated lean mice, 115 mg/dL for ISIS 180272-treated lean mice and 100 mg/dL for ISIS 180280-treated lean mice. Triglycerides were approximately 155 mg/dL for saline treated lean mice and substantially reduced to 100 mg/dL for ISIS 180272-treated lean mice and 90 mg/dL for ISIS 180280-treated lean mice.

Glucocorticoid receptor mRNA levels in liver were measured at the end of study using RiboGreen™ RNA quantification reagent (Molecular Probes, Inc. Eugene, Oreg.) as taught in previous examples above. Glucocorticoid receptor mRNA levels were reduced by approximately 45% in lean mice treated with ISIS 180272, and by approximately 25% in lean mice treated with ISIS 180280, when compared to saline treatment.

### Example 18

### Effect of Antisense Inhibitors of Glucocorticoid Receptor on Ob/Ob Mice

Ob/ob mice have a mutation in the leptin gene which results in obesity and hyperglycemia. As such, these mice are a useful model for the investigation of obesity and diabetes and treatments designed to treat these conditions. In accordance with the present invention, compounds targeted to glucocorticoid receptor are tested in the ob/ob model of obesity and diabetes.

Seven-week old male C57B1/6J-Lep ob/ob mice (Jackson Laboratory, Bar Harbor, Me.) are fed a diet with a fat content of 10-15% and are subcutaneously injected with oligonucleotides at a dose of 25 mg/kg two times per week for 5 weeks. Glucocorticoid antisense oligonucleotides used were ISIS 180272 (SEQ ID NO: 69) and ISIS 180280 (SEQ ID NO: 77). ISIS 116847 (CTGCTAGCCTCTGGATTTGA; SEQ ID NO: 304), targeted to mouse PTEN, was used as a positive control and ISIS 141923 (CCTTCCCTGAAGGTTCCTCC; SEQ ID NO: 305), an unrelated oligonucleotide, was used as the negative oligonucleotide control. Saline-injected animals also serve as controls.

To assess the physiological effects resulting from antisense inhibition of target mRNA, the ob/ob mice that receive antisense oligonucleotide treatment are further evaluated at the end of the treatment period for serum lipids, serum free fatty acids, serum cholesterol, liver triglycerides, fat tissue triglycerides and liver enzyme levels. Hepatic steatosis, or clearing of lipids from the liver, is assessed by measuring the liver triglyceride content. Hepatic steatosis is assessed by routine histological analysis of frozen liver tissue sections stained with oil red O stain, which is commonly used to visualize lipid deposits, and counterstained with hematoxylin and eosin, to visualize nuclei and cytoplasm, respectively.

The effects of target inhibition on glucose and insulin metabolism are evaluated in the ob/ob mice treated with antisense oligonucleotides. Plasma glucose is measured prior to antisense oligonucleotide treatment (day −1) and following two and four weeks of treatment (day 12 and 27, respectively). Both fed and fasted plasma glucose levels are measured. Plasma insulin is also measured at the beginning of the treatment, and following 2 weeks and 4 weeks of treatment. Glucose and insulin tolerance tests are also administered in fed and fasted mice. Mice receive intraperitoneal injections of either glucose or insulin, and the blood glucose and insulin levels are measured before the insulin or glucose challenge and at 15, 20 or 30 minute intervals for up to 3 hours.

In ob/ob mice treated with ISIS 180272 (SEQ ID NO: 69), an antisense inhibitor of glucocorticoid receptor, fed plasma glucose levels were approximately 345 mg/dL at day −1, 350 mg/dL at day 12 and 245 mg/dL at day 27. In mice treated with ISIS 180280 (SEQ ID NO: 77), another antisense inhibitor of glucocorticoid receptor, fed plasma glucose levels were approximately 350 mg/dL at day −1, 340 mg/dL at day 12 and 255 mg/dL at day 27. In contrast, mice treated with saline alone had fed plasma glucose levels of approximately 350 mg/dL at day −1, 420 mg/dL at day 12 and 400 mg/dL at day 27. Mice treated with a positive control oligonucleotide, ISIS 116847 (SEQ ID NO: 304), targeted to PTEN, had fed plasma glucose levels of approximately 340 mg/dL at day −1, 230 mg/dL at day 12 and 200 mg/dL at day 27. Mice treated with negative control oligonucleotide ISIS 141923 had fed plasma glucose levels of approximately 360 mg/dL at day −1, 480 mg/dL at day 12 and 430 mg/dL at day 27. Thus fed plasma glucose levels were reduced after treatment with antisense inhibitors of glucocorticoid receptor. Hypoglycemia was not seen.

In fasted ob/ob mice, plasma glucose levels were measured on day 19 (after a 16 hour fast) and day 29 (after a 12 hour fast). In ob/ob mice treated with ISIS 180272 (SEQ ID NO: 69), an antisense inhibitor of glucocorticoid receptor, fasted plasma glucose levels were approximately 190 mg/dL at day 19 and 220 mg/dL at day 29. In mice treated with ISIS 180280 (SEQ ID NO: 77), another antisense inhibitor of glucocorticoid receptor, fasted plasma glucose levels were approximately 195 mg/dL at day 19 and 270 mg/dL at day 29. In contrast, mice treated with saline alone had fasted plasma glucose levels of approximately 320 mg/dL at day 19 and 320 mg/dL at day 29. Mice treated with a positive control oligonucleotide, ISIS 116847 (SEQ ID NO: 304), targeted to PTEN, had fasted plasma glucose levels of approximately 170 mg/dL at day 19 and 190 mg/dL at day 29. Mice treated with negative control oligonucleotide ISIS 141923 had fasted plasma glucose levels of approximately 245 mg/dL at day 19 and 340 mg/dL at day 29. Thus fasted plasma glucose levels were also reduced after treatment with antisense inhibitors of glucocorticoid receptor. Hypoglycemia was not observed.

Serum lipids in ob/ob mice were also measured at the end of the study. Cholesterol levels were approximately 270 mg/dL for saline treated mice, 305 mg/dL for ISIS 180272-treated mice, 250 mg/dL for ISIS 180280-treated mice, 285 mg/dL for ISIS 116847-treated mice and 265 mg/dL for ISIS 141923-treated mice. Triglycerides were approximately 120 mg/dL for saline treated mice, 115 mg/dL for ISIS 180272-treated mice, 105 mg/dL for ISIS 180280-treated mice, 95 mg/dL for ISIS 116847-treated mice and 95 mg/dL for ISIS 141923-treated mice.

Glucocorticoid receptor mRNA levels in ob/ob mouse livers were measured at the end of study using RiboGreen™ RNA quantification reagent (Molecular Probes, Inc. Eugene, Oreg.) as taught in previous examples above. Glucocorticoid receptor mRNA levels were reduced by approximately 80% in mice treated with ISIS 180272, and by approximately 70% in mice treated with ISIS 180280, when compared to saline treatment. Glucocorticoid receptor mRNA levels were not significantly decreased in mice treated with the positive control oligonucleotide, ISIS 116847, and were slightly increased (120% of control) in mice treated with the negative control oligonucleotide, ISIS 141923.

Glucocorticoid receptor mRNA levels in ob/ob mouse white adipose tissue were measured at the end of study using RiboGreen™ RNA quantification reagent (Molecular Probes, Inc. Eugene, Oreg.) as taught in previous examples above. Glucocorticoid receptor mRNA levels in fat were reduced by approximately 40% in mice treated with ISIS 180272, and by approximately 52% in mice treated with ISIS 180280, when compared to saline treatment. Glucocorticoid receptor mRNA levels in fat were slightly decreased (by approx. 13%) in mice treated with the positive control oligonucleotide, ISIS 116847, and were slightly increased (120% of control) in mice treated with the negative control oligonucleotide, ISIS 141923.

### Example 19

### Effect of Antisense Inhibitors of Glucocorticoid Receptor in Leptin Receptor-Deficient Mice (Db/Db Mice)

Leptin is a hormone produced by fat that regulates appetite. Deficiencies in this hormone in both humans and non-human animals lead to obesity. db/db mice have a mutation in the leptin receptor gene which results in obesity and hyperglycemia. As such, these mice are a useful model for the investigation of obesity and diabetes and treatments designed to treat these conditions. db/db mice, which have lower circulating levels of insulin and are more hyperglycemic than ob/ob mice which harbor a mutation in the leptin gene, are often used as a rodent model of type 2 diabetes. In accordance with the present invention, oligomeric compounds of the present invention are tested in the db/db model of obesity and diabetes.

Seven-week old male C57B1/6J-Lepr db/db mice (Jackson Laboratory, Bar Harbor, Me.) were fed a diet with a fat content of 15-20% and are subcutaneously injected with one or more of the oligomeric compounds of the invention or a control compound at a dose of 25 mg/kg two times per week for 5 weeks. Glucocorticoid antisense oligonucleotides used were ISIS 180272 (SEQ ID NO: 69) and ISIS 180280 (SEQ ID NO: 77). ISIS 116847 (SEQ ID NO: 304), targeted to mouse PTEN, was used as a positive control and ISIS 141923 (SEQ ID NO: 305), an unrelated oligonucleotide, was used as the negative oligonucleotide control. Oligonucleotides were prepared in buffered saline and sterilized by filtration through a 0.2 micron filter. Saline-injected animals, leptin receptor wildtype littermates (i.e. lean littermates) and db/db mice fed a standard rodent diet serve as controls. After the treatment period, mice are sacrificed and target levels are evaluated in liver, brown adipose tissue (BAT) and white adipose tissue (WAT). RNA isolation and target mRNA expression level quantitation are performed as described by other examples herein.

To assess the physiological effects resulting from inhibition of target mRNA, the db/db mice are further evaluated at the end of the treatment period for serum triglycerides, serum lipoproteins, serum free fatty acids, serum cholesterol, serum apolipoproteins, liver tissue triglycerides, fat tissue triglycerides and serum transaminase levels. Triglycerides, lipoproteins, cholesterol and transaminases are measured by routine clinical analyzer instruments (e.g. Olympus Clinical Analyzer, Melville, N.Y.). Serum free fatty acids are measured using a Wako Chemicals kit for non-esterified free fatty acids (Richmond, Va.). Tissue triglyceride levels are measured using a Triglyceride GPO Assay from Roche Diagnostics (Indianapolis, Ind.). Liver triglyceride levels are used to assess hepatic steatosis, or clearing of lipids from the liver. Hepatic steatosis is also assessed by routine histological analysis of frozen liver tissue sections stained with oil red O stain, which is commonly used to visualize lipid deposits, and counterstained with hematoxylin and eosin, to visualize nuclei and cytoplasm, respectively.

The effects of target inhibition on glucose and insulin metabolism are evaluated in the db/db mice treated with the oligomeric compounds of the invention. Plasma glucose (fed and fasted) is measured at the start of the treatment and weekly during treatment. Plasma insulin is similarly measured. Glucose and insulin tolerance tests are also administered in fed and fasted mice. Mice receive intraperitoneal injections of either glucose or insulin, and the blood glucose and insulin levels are measured before the insulin or glucose challenge and at 15, 20 or 30 minute intervals for up to 3 hours. Glucose levels are measured using a YSI glucose analyzer (YSI Scientific, Yellow Springs, Ohio) and insulin levels are measure using an Alpco insulin-specific ELISA kit from (Windham, N.H.).

In db/db mice treated with ISIS 180272 (SEQ ID NO: 69), an antisense inhibitor of glucocorticoid receptor, fed plasma glucose levels were approximately 300 mg/dL at day −1, 445 mg/dL at day 5, 450 mg/dL at day 12 and 450 mg/dL at day 26. In mice treated with ISIS 180280 (SEQ ID NO: 77), another antisense inhibitor of glucocorticoid receptor, fed plasma glucose levels were approximately 300 mg/dL at day −1, 480 mg/dL at day 5, 440 mg/dL at day 12 and 480 mg/dL at day 26. Mice treated with saline alone had fed plasma glucose levels of approximately 300 mg/dL at day −1, 470 mg/dL at day 5, 510 mg/dL at day 12 and 500 mg/dL at day 26. db/db mice treated with a positive control oligonucleotide, ISIS 116847 (SEQ ID NO: 304), targeted to PTEN, had fed plasma glucose levels of approximately 300 mg/dL at day −1, 405 mg/dL at day 5, 300 mg/dL at day 12 and 350 mg/dL at day 26. Mice treated with negative control oligonucleotide ISIS 141923 had fed plasma glucose levels of approximately 300 mg/dL at day −1, 405 mg/dL at day 5, 425 mg/dL at day 12 and 500 mg/dL at day 26.

In fasted db/db mice, plasma glucose levels were measured on day 19 (after a 16 hour fast) and day 29 (after a 12 hour fast). In db/db mice treated with ISIS 180272 (SEQ ID NO: 69), an antisense inhibitor of glucocorticoid receptor, fasted plasma glucose levels were approximately 200 mg/dL at day 19 and 210 mg/dL at day 29. In mice treated with ISIS 180280 (SEQ ID NO: 77), another antisense inhibitor of glucocorticoid receptor, fasted plasma glucose levels were approximately 260 mg/dL at day 19 and 235 mg/dL at day 29. In contrast, mice treated with saline alone had fasted plasma glucose levels of approximately 320 mg/dL at day 19 and 300 mg/dL at day 29. Mice treated with a positive control oligonucleotide, ISIS 116847 (SEQ ID NO: 304), targeted to PTEN, had fasted plasma glucose levels of approximately 320 mg/dL at day 19 and 195 mg/dL at day 29. Mice treated with negative control oligonucleotide ISIS 141923 had fasted plasma glucose levels of approximately 300 mg/dL at day 19 and 260 mg/dL at day 29. Thus fasted plasma glucose levels were reduced after treatment with antisense inhibitors of glucocorticoid receptor and hypoglycemia was not seen.

Serum lipids in db/db mice were also measured at the end of the study. Cholesterol levels were approximately 170 mg/dL for saline treated mice, 125 mg/dL for ISIS 180272-treated mice, 120 mg/dL for ISIS 180280-treated mice, 150 mg/dL for ISIS 116847-treated mice and 195 mg/dL for ISIS 141923-treated mice. Triglycerides were approximately 220 mg/dL for saline treated mice, 95 mg/dL for ISIS 180272-treated mice, 105 mg/dL for ISIS 180280-treated mice, 105 mg/dL for ISIS 116847-treated mice and 245 mg/dL for ISIS 141923-treated mice. Serum lipids, especially triglycerides, were thus decreased in db/db mice treated with antisense inhibitors of glucocorticoid receptors.

A second experiment was conducted similarly to that described above in this example, with fed plasma glucose measurements conducted on days −1, 6, 13 and 26. In these db/db mice treated with ISIS 180272 (25 mg/kg×2 weekly s.c.; SEQ ID NO: 69), an antisense inhibitor of glucocorticoid receptor, fed plasma glucose levels were approximately 260 mg/dL at day −1, 290 mg/dL at day 6, 405 mg/dL at day 13 and 305 mg/dL at day 26. In mice treated with ISIS 180280 (25 mg/kg×2 weekly s.c.; SEQ ID NO: 77), another antisense inhibitor of glucocorticoid receptor, fed plasma glucose levels were approximately 260 mg/dL at day −1, 375 mg/dL at day 6, 345 mg/dL at day 13 and 355 mg/dL at day 26. Mice treated with saline alone had fed plasma glucose levels of approximately 260 mg/dL at day −1, 465 mg/dL at day 6, 480 mg/dL at day 13 and 510 mg/dL at day 26. db/db mice treated with a positive control oligonucleotide, ISIS 116847 (SEQ ID NO: 304), targeted to PTEN, had fed plasma glucose levels of approximately 250 mg/dL at day—1, 355 mg/dL at day 6, 325 mg/dL at day 13 and 225 mg/dL at day 26. Mice treated with negative control oligonucleotide ISIS 141923 had fed plasma glucose levels of approximately 260 mg/dL at day −1, 430 mg/dL at day 6, 402 mg/dL at day 13 and 480 mg/dL at day 26.

Serum lipids in db/db mice were also measured at the end of the study. Cholesterol levels were approximately 170 mg/dL for saline treated mice, 190 mg/dL for ISIS 180272-treated mice, 155 mg/dL for ISIS 180280-treated mice, 180 mg/dL for ISIS 116847-treated mice and 185 mg/dL for ISIS 141923-treated mice. Triglycerides were approximately 220 mg/dL for saline treated mice, 120 mg/dL for ISIS 180272-treated mice, 115 mg/dL for ISIS 180280-treated mice, 190 mg/dL for ISIS 116847-treated mice and 180 mg/dL for ISIS 141923-treated mice. Serum triglycerides were thus decreased in db/db mice treated with antisense inhibitors of glucocorticoid receptors.

Glucocorticoid receptor mRNA levels in db/db mouse livers were measured at the end of study using RiboGreen™ RNA quantification reagent (Molecular Probes, Inc. Eugene, Oreg.) as above. Glucocorticoid receptor mRNA levels were reduced by approximately 53% in mice treated with ISIS 180272, and by approximately 65% in mice treated with ISIS 180280, when compared to saline treatment. Glucocorticoid receptor mRNA levels were not decreased in mice treated with the positive control oligonucleotide, ISIS 116847 or the negative control oligonucleotide, ISIS 141923.

Plasma corticosterone levels were measured in these mice as a marker for stimulation of the hypothalamic pituitary axis. Corticosterone levels (measured by ELISA kit, ALPCO, Windham N. H., following manufacturer's instructions) were 180 ng/ml in mice treated with saline; 225 ng/ml in mice treated with the PTEN inhibitor ISIS 116847, 175 ng/ml in mice treated with the glucocorticoid receptor inhibitor ISIS 180272, 170 ng/ml in mice treated with the glucocorticoid receptor inhibitor ISIS 180280 and 220 ng/ml in mice treated with control oligonucleotide ISIS 141923.

### Example 20

### Effect of Antisense Inhibitors of Glucocorticoid Receptor on Lean (ZDF+/−) Rats

ZDF+/− rats are heterozygous littermates of Zucker Diabetic Fatty rats, often referred to as lean littermates because they do not display the impaired insulin sensitivity phenotype of the homozygous ZDF fa/fa rat. Homogygous ZDF rats harbor a mutation in the leptin receptor which makes them a useful animal model of impaired insulin sensitivity.

Six week old ZDF+/− (lean) male rats were dosed twice weekly with 37.5 mg/kg of antisense oligonucleotide, given subcutaneously. Oligonucleotides were prepared in buffered saline and filter-sterilized. A total of five doses were given. Glucocorticoid antisense oligonucleotides used were ISIS 180277 (SEQ ID NO: 74) and ISIS 180281 (SEQ ID NO: 78), targeted to rat glucocorticoid receptor. Plasma glucose levels were measured before the initial dose in week 1 and the day before the rats were sacrificed in week 3. After sacrifice, serum lipids were measured and target reduction in liver was also measured.

In lean ZDF+/− rats treated with ISIS 180277 (SEQ ID NO: 74), an antiscnsc inhibitor of rat glucocorticoid receptor, plasma glucose levels were approximately 170 mg/dL at week 1 and 120 mg/dL at week 3. In lean ZDF+/− rats treated with ISIS 180281 (SEQ ID NO: 78), another antisense inhibitor of glucocorticoid receptor, plasma glucose levels were approximately 140 mg/dL at week 1 and 140 mg/dL at week 3. Lean rats treated with saline alone had fed plasma glucose levels of approximately 142 mg/dL at week 1 and 135 mg/dL at week 3. While plasma glucose levels decreased with 180277 treatment, the rats did not become hypoglycemic.

Serum lipids were also measured at the end of the study. Cholesterol levels were approximately 110 mg/dL for saline treated lean rats, 70 mg/dL for ISIS 180277-treated lean rats and 25 mg/dL for ISIS 180281-treated lean rats. Triglycerides were approximately 115 mg/dL for saline treated lean rats and substantially reduced to approximately 15 mg/dL for ISIS 180277-treated lean rats and 20 mg/dL for ISIS 180281-treated lean rats.

Glucocorticoid receptor mRNA levels in lean ZDF+/− rat livers were measured at the end of study using RiboGreen™ RNA quantification reagent (Molecular Probes, Inc. Eugene, Oreg.) as taught in previous examples above. Glucocorticoid receptor mRNA levels were reduced by approximately 59% in rats treated with ISIS 180277, and by approximately 65% in rats treated with ISIS 180281, when compared to saline treatment.

### Example 21

### Effect of Antisense Inhibitors of Glucocorticoid Receptor on Zucker Diabetic Fatty (ZDF) Rats

The leptin receptor deficient (fa/fa) Zucker diabetic fatty (ZDF) rat is another useful model for the investigation of type 2 diabetes. Diabetes develops spontaneously in these male rats at ages 8-10 weeks, and is associated with hyperphagia, polyuria, polydipsia, and impaired weight gain, symptoms which parallel the clinical symptoms of diabetes. Phillips M S, Liu Q, Hammond H A, Dugan V, Hey P J, Caskey C J, Hess J F, 1996, Nat Genet. 13, 18-19.

Six week old ZDF male rats were subcutaneously injected with oligonucleotides at a dose of 37.5 mg/kg two times per week for 7 weeks. Glucocorticoid antisense oligonucleotides used were ISIS 180277 (SEQ ID NO: 74) and ISIS 180281 (SEQ ID NO: 78). ISIS 116847 (GCGACAGCTGCTCCACCTTC; SEQ ID NO: 304), targeted to rat, mouse and human PTEN, was used as a positive control and ISIS 141923 (CCTTCCCTGAAGGTTCCTCC; SEQ ID NO: 305), an unrelated oligonucleotide, was used as the negative oligonucleotide control. Saline-injected animals also serve as controls. Oligonucleotides were prepared in buffered saline and filter-sterilized.

In ZDF rats treated with ISIS 180277 (SEQ ID NO: 74), an antisense inhibitor of glucocorticoid receptor, fed plasma glucose levels were approximately 210 mg/dL at day −1, 280 mg/dL at day 6, 390 mg/dL at day 13, 395 mg/dL at day 26 and 420 mg/dL at day 40. In rats treated with ISIS 180281 (SEQ ID NO: 78), another antisense inhibitor of glucocorticoid receptor, fed plasma glucose levels were approximately 210 mg/dL at day −1, 290 mg/dL at day 6, 395 mg/dL at day 13, 410 mg/dL at day 26 and 435 mg/dL at day 40. In contrast, rats treated with saline alone had fed plasma glucose levels of approximately 210 mg/dL at day −1, 260 mg/dL at day 6, 405 mg/dL at day 13, 410 mg/dL at day 26 and 445 mg/dL at day 40. Rats treated with a positive control oligonucleotide, ISIS 116847 (SEQ ID NO: 304), targeted to PTEN, had fed plasma glucose levels of approximately 210 mg/dL at day −1, 190 mg/dL at day 6, 150 mg/dL at day 13, 110 mg/dL at day 26 and 130 mg/dL at day 40.

In fasted ZDF rats, plasma glucose levels were measured on day 21 (after a 16 hour fast) and day 33 (after a 12 hour fast). In rats treated with ISIS 180277 (SEQ ID NO: 74), an antisense inhibitor of glucocorticoid receptor, fasted plasma glucose levels were approximately 145 mg/dL at day 21 and 130 mg/dL at day 33. In mice treated with ISIS 180281 (SEQ ID NO: 78), another antisense inhibitor of glucocorticoid receptor, fasted plasma glucose levels were approximately 170 mg/dL at day 19 and 140 mg/dL at day 29. In contrast, rats treated with saline alone had fasted plasma glucose levels of approximately 270 mg/dL at day 19 and 255 mg/dL at day 29. Rats treated with a positive control oligonucleotide, ISIS 116847 (SEQ ID NO: 304), targeted to PTEN, had fasted plasma glucose levels of approximately 115 mg/dL at day 19 and 120 mg/dL at day 29. Thus fasted plasma glucose levels were reduced after treatment with antisense inhibitors of glucocorticoid receptor. Hypoglycemia was not observed.

Serum lipids in ZDF fa/fa rats were also measured at the end of the study. Cholesterol levels were approximately 190 mg/dL for saline treated rats, 130 mg/dL for ISIS 180277-treated rats, 70 mg/dL for ISIS 180281-treated rats and 140 mg/dL for ISIS 116847-treated rats. Triglycerides were approximately 520 mg/dL for saline treated rats, 360 mg/dL for ISIS 180277-treated rats, 125 mg/dL for ISIS 180281-treated rats and 910 mg/dL for ISIS 116847-treated rats. Thus both antisense inhibitors of glucocorticoid receptor had lipid-lowering effects.

Serum free fatty acids were also measured in ZDF rats after antisense treatment. Free fatty acids were approximately 0.68 mEq/1 for saline-treated rats, 0.48 mEq/1 for ISIS 180277-treated rats and 0.31 mEq/l for ISIS 180281-treated rats.

A reduction in epididymal fat pad weights by glucocorticoid receptor antisense oligonucleotide was also observed in ZDF rats (saline 3.8±0.07 grams vs. antisense 2.6±0.06 grams p<0.05). The effects of glucocorticoid receptor antisense inhibition were not accompanied by any changes in food intake or body weight in these animals. To understand the mechanism underlying the lipid lowering effects of the glucocorticoid receptor antisense oligonucleotide, the expression of several lipogenic genes was investigated in these models. Glucocorticoid receptor antisense treatment caused a reduction in the expression of 3-hydroxy-3-methylglutaryl-coenzyme A (HMG-CoA) reductase, a rate limiting enzyme in cholesterol biosynthesis, thus explaining in part the effects of the glucocorticoid receptor antisense compound on cholesterol levels. The expression of several other lipogenic genes, including squalene synthase, sterol regulatory element binding protein-1c (SREBP-1c), HMGCoA synthase, remained unchanged.

Liver enzymes (AST/ALT) were also measured in these rats as a marker for liver toxicity. Liver enzymes were not increased by antisense treatment and actually decreased compared to saline-treated animals.

Glucocorticoid receptor mRNA levels in ZDF fa/fa rat livers and white adipose tissue were measured at the end of study using RiboGreen™ RNA quantification reagent (Molecular Probes, Inc. Eugene, Oreg.) as taught in previous examples above. Glucocorticoid receptor mRNA levels were reduced by approximately 60% in livers of rats treated with ISIS 180277, and by approximately 63% in rats treated with ISIS 180281, when compared to saline treatment. Glucocorticoid receptor mRNA levels were also reduced by approximately 60% in fat of rats treated with ISIS 180281.

### Example 22

### Effect of Antisense Inhibitors of Glucocorticoid Receptor in the High Fat Diet/Streptozotocin (HFD/STZ) Rat Model

The HFD/STZ rat model (based on Reed et al., 2000, Metabolism, 49, 1390-1394) closely mimics human type 2 diabetes with the dietary component induced by the high fat diet (also known as DIO or diet-induced obesity) and hyperglycemia induced by streptozotocin. Unlike the ob/ob and db/db models, the diabetes phenotype is not caused by mutations of either the leptin or leptin receptor gene.

Seven week old male Sprague Dawley rats (weighing 160-180 grams) were fed with high fat diet consisting of 40% fat, 41% carbohydrate and 18% protein (Harlan Teklad Adjusted Fat Diet 96132, Harlan Teklad, Madison Wis.) for 2 weeks. During this feeding schedule, animals were monitored for glucose, insulin, triglycerides, and free fatty acids from plasma samples obtained by tail bleeding. HFD treatment when compared to normal chow treatment, resulted in significant elevation of insulin, triglycerides and free fatty acids but no increase in plasma glucose indicating that the animals had acquired the insulin resistance phenotype, which was further confirmed by oral glucose tolerance test. In order to induce hyperglycemia, the rats were injected intraperitoneally with 50 mg/kg of freshly prepared STZ. At this dose, STZ causes a moderate destruction of insulin-producing β-cells in the pancreas resulting in hyperglycemia measured after 3 days. Animals with plasma glucose values ranging between 350-450 mg/dL were selected to test the therapeutic effects of antisense drugs. In the first study, antisense oligonucleotide inhibitors of PTEN and glucocorticoid receptor were tested in this model. These compounds were administered by subcutaneous route with doses 25 mg/kg twice weekly for 4 weeks. Oligonucleotides were prepared in buffered saline and filter-sterilized. Plasma glucose and triglycerides were measured weekly.

In HFD/STZ rats treated with ISIS 180281 (SEQ ID NO: 78), an antisense inhibitor of glucocorticoid receptor, fed plasma glucose levels were 450 mg/dL at week 1, 487 mg/dL at week 2, and 446 mg/dL at week 4. Rats treated with saline alone had fed plasma glucose levels of approximately 432 mg/dL at week 1, 470 mg/dL at week 2, and 487 mg/dL at week 4. Rats treated with a positive control oligonucleotide, ISIS 116847 (SEQ ID NO: 304), targeted to PTEN, had fed plasma glucose levels of 446 mg/dL at week 1, 552 mg/dL at week 2 and 398 mg/dL at week 4. Rats treated with negative control oligonucleotide ISIS 141923 had fed plasma glucose levels of approximately 443 mg/dL at week 1, 525 mg/dL at week 2 and 398 mg/dL at week 4.

In fasted HFD/STZ rats, plasma glucose levels were 100 mg/dL in rats treated with ISIS 180281 (SEQ ID NO: 78), an antisense inhibitor of glucocorticoid receptor, 320 mg/dL in rats treated with saline alone, 155 mg/dL in rats treated with a positive control oligonucleotide, ISIS 116847 (SEQ ID NO: 304), targeted to PTEN, and 225 mg/dL in rats treated with negative control oligonucleotide ISIS 141923. Thus fasted plasma glucose levels were reduced after treatment with an antisense inhibitor of glucocorticoid receptor.

Serum lipids in HFD/STZ rats were also measured at the end of the study. Cholesterol levels were approximately 100 mg/dL for saline treated rats, 50 mg/dL for ISIS 180281-treated rats, 75 mg/dL for ISIS 116847-treated rats and 95 mg/dL for ISIS 141923-treated rats. Triglycerides were approximately 230 mg/dL for saline treated rats, 30 mg/dL for ISIS 180281-treated rats, 125 mg/dL for ISIS 116847-treated rats and 175 mg/dL for ISIS 141923-treated rats. Thus antisense inhibition of glucocorticoid receptor had lipid-lowering effects. Glucocorticoid receptor antisense also significantly reduced plasma free fatty acids in HFD-STZ rats (saline 0.93±0.12 mEQ/L vs. antisense 0.52±0.04 mEQ/L, p<0.05).

Plasma corticosterone levels were measured in these STZ-HFD rats and were unchanged by treatment with antisense inhibitor of glucocorticoid receptor, compared to saline treatment.

A reduction in epididymal fat pad weights by glucocorticoid receptor antisense oligonucleotide was also observed in HFD-STZ rats (saline 2.41±0.23 grams vs. antisense 1.8±0.63 grams). The effects of glucocorticoid receptor antisense inhibition were not accompanied by any changes in food intake or body weight in these animals. To understand the mechanism underlying the lipid lowering effects of the glucocorticoid receptor antisense oligonucleotide, we investigated the expression of several lipogenic genes in these models. Glucocorticoid receptor antisense treatment caused a reduction in the expression of 3-hydroxy-3-methylglutaryl-coenzyme A (HMG-CoA) reductase, a rate limiting enzyme in cholesterol biosynthesis, thus explaining in part the effects of the glucocorticoid receptor antisense compound on cholesterol levels. The expression of several other lipogenic genes, including squalene synthase, sterol regulatory element binding protein-1c (SREBP-1c), HMGCoA synthase, remained unchanged.

Glucocorticoid receptor levels in liver were also measured following treatment. Compared to saline controls, glucocorticoid mRNA levels were reduced by 50% by ISIS 180281 (SEQ ID NO: 78) and were not reduced at all by ISIS 141923. The PTEN antisense oligonucleotide, ISIS 116847 caused no inhibition and in fact caused a slight increase in glucocorticoid receptor mRNA levels (121% of saline control).

Thus treatment with antisense to glucocorticoid receptor resulted in 50% reduction in glucocorticoid receptor expression in the liver, approximately 50% decrease in fasted glucose and 68% reduction in plasma glucose. These results are consistent with the data that were obtained in Zucker Diabetic Fatty (ZDF) rat model.

### Example 23

### Effects of Antisense Inhibition of Glucocorticoid Receptor on Hepatic and Systemic Response to Glucocorticoids

Glucocorticoid receptor antagonists that have systemic inhibitory effects can cause undesirable side effects, such as stimulation of the hypothalamic pituitary axis. A significant advantage of antisense inhibitors is that reduced expression of the glucocorticoid receptor by antisense oligonucleotides is observed to a large extent in liver and fat, which are desirable target tissues for inhibition of glucocorticoid receptor, but to a lesser extent systemically. Thus there is believed to be reduced likelihood of undesirable systemic side effects (primarily stimulation of the hypothalamic pituitary axis) with oligonucleotide inhibitors, in comparison to other classes of inhibitors of this target.

To confirm that glucocorticoid receptor expression could be modulated in the liver without being modulated in the pituitary, ZDF rats were treated with the antisense inhibitor of glucocorticoid receptor (ISIS 180281) at 37.5 mg/kg administered subcutaneously twice a week for 5 weeks. RNA was isolated from the pituitary gland and glucocorticoid receptor levels were measured by Ribogreen™ analysis as described in previous examples. In rats treated with antisense to glucocorticoid receptor, levels of glucocorticoid receptor in the pituitary were identical to those in saline-treated rats. Thus it can be shown that antisense inhibition of this target can be achieved in specific organs (e.g., liver) which would lead to desired effects, without inhibition in undesirable site of inhibition for this target (e.g., pituitary). Pituitary gland proopiomelanocortin (POMC-1) RNA expression was also measured by RT-PCR and was not significantly affected by antisense inhibitors of glucocorticoid receptor expression.

Corticosterone levels were also measured as a marker for stimulation of the hypothalamic pituitary axis. No significant changes in corticosterone levels were seen (35 ng/ml for ZDF rats treated with antisense to glucocorticoid receptor, vs 45 ng/ml for saline treated rats and 30 ng/ml for control oligonucleotide-treated rats). Corticosterone levels were similarly found to be unchanged in mice treated with antisense inhibitors of glucocorticoid receptor.

To further test this hypothesis, normal male Sprague Dawley rats were treated with antisense inhibitor of glucocorticoid receptor (ISIS 180281; SEQ ID NO: 78) or a control oligonucleotide (ISIS 141923; SEQ ID NO: 305) at a dose of 50 mg/kg twice a week for 4 weeks. Subsequently, the animals were fasted overnight and were injected with saline or dexamethasone (4 mg/kg i.p.). Four hours after the injection, the animals were sacrificed and liver tissue was harvested to examine changes in tyrosine aminotransferase/TAT mRNA expression (a steroid responsive gene that was used as a marker of hepatic steroid activity) by RT-PCR. In addition, blood was sampled for measurement of circulating lymphocytes, which is a marker of systemic glucococorticoid effects. As expected, dexamethasone caused a significant increase in TAT expression and a decrease in circulating lymphocytes (i.e., lymphopenia). Treatment with the glucocorticoid receptor antisense inhibitor led to about a 75% reduction in hepatic glucocorticoid receptor expression, which was accompanied by a prevention of dexamethasone induced increase in TAT expression. However, no effect was observed on the dexamethasone-induced decrease in circulating lymphocytes (i.e., no lymphopenia).

These results along with the lack of effect on corticosterone levels in the previous example indicate that antisense inhibition of glucocorticoid receptor expression leads to functional antagonism of glucocorticoid effects in the liver without altering systemic glucocorticoid effects.

Levels of plasma adrenocorticotropic hormone (ACTH) were also examined after dexamethasone challenge. ACTH levels were measured using an ELISA kit (ALPCO, Windham N.H.) according to manufacturer's instructions. Glucocorticoid antisense oligonucleotide (ISIS 180281) did not affect basal ACTH levels (saline 9.79 pg/ml vs ASO 9.96 pg/ml). ISIS 180281 also reduced dexamethasone-induced PEPCK expression in the liver. Liver glycogen after dexamethasone challenge was also changed by antisense treatment with ISIS 180281. In mice treated with ISIS 180281, liver glycogen levels were approximately 1.5 mg/g in animals given dexamethasone challenge, compared to approx 1 mg/g in animals given saline in place of dexamethasone. In mice treated with the control oligonucleotide ISIS 141923, liver glycogen levels were approximately 13.5 mg/g in animals given dexamethasone challenge, compared to approximately 4 mg/g in animals given saline in place of dexamethasone. In mice treated with saline (no oligonucleotide), liver glycogen levels were approximately 9 mg/g in animals given dexamethasone challenge, compared to approx 3 mg/g in animals given saline in place of dexamethasone.

### Example 24

### Effect of Antisense Inhibitors of Glucocorticoid Receptor on Diet-Induced Obesity in Rats

Antisense inhibitors of glucocorticoid receptor expression are expected to reduce obesity or weight gain. This is tested in the high fat diet (HFD) model, also known as the DIO (diet-induced obesity) model.

Seven week old male Sprague Dawley rats (weighing 160-180 grams) are fed with high fat diet consisting of 40% fat, 41% carbohydrate and 18% protein (Harlan Teklad Adjusted Fat Diet 96132) for 2 weeks. During this feeding schedule, animals are weighed at regular intervals.

Mice treated with an antisense inhibitor of glucocorticoid receptor gained less weight on the high fat diet than did saline-treated animals. Therefore the antisense-treated mice are less likely to be obese.

### Example 25

### Glucocorticoid Receptor Antisense Inhibition Decreases Hepatic Glucose Production and Gluconeogenesis

Ex vivo hepatic glucose production was measured in liver slices from Sprague Dawley rats treated with glucocorticoid receptor antisense oligonucleotide (ISIS 180281). Control and antisense-treated rats were fasted for 24 hours and administered either vehicle or dexamethasone (Bausch & Lomb, Tampa, Fla.) at a dose of 12.5 mg/kg. Six hours after treatment, precision-cut liver slices were prepared using a Krumdieck Tissue Slicer (Alabama Research and Development, Munford, Ala.), and incubated in glucose-free DMEM (Invitrogen, Carlsbad, Calif.) supplemented with 0.1% BSA, 10 mM lactate, 1 mM sodium pyruvate, 10 mM alanine, and 10 mM glycerol (NGS medium). After a 1 hour pre-incubation, individual slices were transferred to separate wells of a 24-well plate containing 0.5 ml NGS medium, and glucose released into the medium after 1.5 hours was determined by a Hitachi 912 clinical chemistry analyzer. Liver slices were weighed, and glucose production per milligram of liver tissue was determined.

Rats treated with antisense to glucocorticoid receptor (ISIS 180281) showed a significant reduction in basal glucose production (control antisense compound 0.86±0.16 vs. 0.35±0.01 glucose (g)/hour/liver slice (mg), p<0.05). To directly evaluate the effects of the antisense compound on glucocorticoid-mediated glucose production, assays were performed in antisense-treated rats that were injected with dexamethasone 6 hours prior to necropsy. Glucocorticoid receptor antisense compound dramatically inhibited dexamethasone-induced glucose production (control antisense+dexamethasone 5.61±0.68 vs. glucocorticoid receptor antisense+dexamethasone 0.61±0.04 glucose (g)/hour/liver slice (mg), p<0.05).

### Example 26

### Additional Cross-Species Glucocorticoid Receptor Antisense Oligonucleotides

Additional candidate antisense inhibitors of mouse glucocorticoid receptor were evaluated in comparison to previously screened oligonucleotides for ability to reduce expression of glucocorticoid receptor mRNA in vivo in various species. This is shown in Table 7. Shown are oligonucleotide sequence and position (position of 5′ most nucleobase) on SEQ ID NO: 4 (human glucocorticoid receptor mRNA, GenBank accession no. NM_{—}000176.1). Also shown is complementarity to human, cynomolgus monkey, rat and mouse glucocorticoid receptor mRNA (“yes” means perfect complementarity, and “1 mm” means one mismatch from perfect complementarity).

All compounds shown are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P=S) throughout the oligonucleotide. All cytosine residues are 5-methylcytosines.

TABLE 7
Glucocorticoid receptor cross-species oligonucleotides

| | | | Pos′n | | | | |
|---|---|---|---|---|---|---|---|
| | SEQ | | on SEQ | | | | |
| | ID | | ID NO: | Perfect complement to: | | | |
| ISIS # | NO | Sequence | 4 | Human | Monkey | Rat | Mouse |
| 361137 | 306 | cgacctattgaggtttgcaa | 509 | yes | yes | yes | yes |
| 180276 | 73 | ctttggtctgtggtatacaa | 679 | yes | 1 mm | 1 mm | yes |
| 345198 | 307 | gtcaaaggtgetttggtctg | 689 | yes | yes | yes | yes |
| 180304 | 100 | agcatgtgtttacattggtc | 2053 | yes | yes | yes | yes |
| 180275 | 72 | ctgtggtatacaatttcaca | 672 | yes | 1 mm | 1 mm | yes |
| 361141 | 308 | gcagacattttattaccaat | 1066 | yes | yes | yes | 1 mm |
| 180281 | 78 | gcccagtttctcttgcttaa | 1007 | yes | yes | yes | yes |
| 361151 | 309 | gtacatctgtcctccagagg | 1109 | yes | yes | yes | yes |
| 180274 | 71 | ctggtcgacctattgaggtt | 514 | yes | yes | yes | yes |
| 361156 | 310 | gctgtattcatgtcatagtg | 1129 | yes | yes | yes | yes |

These compounds were tested at a range of doses in cynomolgus monkey and rat primary hepatocytes as well as human cells for inhibition of glucocorticoid receptor expression. Experiments were done twice. IC50s were calculated and are shown in Table 8.

TABLE 8
Glucocorticoid receptor cross species oligonucleotides-IC50s (nM)

| | SEQ | Rat | Rat | Monkey | Monkey | Human | Human |
|---|---|---|---|---|---|---|---|
| | ID | expt 1 | expt 2 | expt 1 | expt 2 | expt 1 | expt 2 |
| ISIS # | NO | IC50 | IC50 | IC50 | IC50 | IC50 | IC50 |
| 361137 | 306 | 19 | 26 | 12 | 11 | 5 | 9 |
| 180276 | 73 | 29 | 38 | 27 | 24 | 5 | 6 |
| 345198 | 307 | 22 | 25 | 35 | 31 | 6 | 11 |
| 180304 | 100 | 32 | 39 | 29 | 28 | 7 | 6 |
| 180275 | 72 | 32 | 41 | 35 | 41 | 9 | 12 |
| 361141 | 308 | 21 | 26 | 15 | 12 | 11 | 15 |
| 180281 | 78 | 21 | 28 | 24 | 22 | 11 | 14 |
| 361151 | 309 | 37 | 51 | 135 | 128 | 12 | 11 |
| 180274 | 71 | 24 | 34 | 105 | 115 | 22 | 24 |
| 361156 | 310 | 22 | 25 | 169 | 158 | 36 | 31 |

Five of these compounds (ISIS 180281, ISIS 180304, ISIS 345198, ISIS 361137 and ISIS 361141) were further tested at various doses in lean (nondiabetic) rats for their ability to reduce glucocorticoid receptor RNA levels in liver. Results are shown in Tables 9a and 9b (separate experiments). Liver enzyme (AST/ALT) levels were also measured in these rats, as a measure of oligonucleotide hepatotoxicity. With the exception of the 50 mg/kg dose of ISIS 180281, none of these compounds caused a significant increase in AST or ALT levels at any dose tested. Rats given a 50 mg/kg dose of ISIS 180281 had both AST and ALT levels nearly twice those of control-treated rats.

TABLE 9a
Rat lean screen—glucocorticoid receptor antisense oligonucleotides

| | % reduction in glucocorticoid receptor mRNA | | |
|---|---|---|---|
| | in rat liver after antisense treatment | | |
| | at doses shown (compared to saline) | | |
| Compound | 50 mg/kg | 25 mg/kg | 12.5 mg/kg |
| ISIS 180281 | 68 | 65 | 48 |
| ISIS 180304 | 52 | 34 | 0 |
| ISIS 345198 | 63 | 58 | 52 |

TABLE 9b
Rat lean screen—glucocorticoid receptor antisense oligonucleotides

| | % reduction in glucocorticoid receptor mRNA | | |
|---|---|---|---|
| | in rat liver after antisense treatment | | |
| | at doses shown (compared to saline) | | |
| Compound | 50 mg/kg | 25 mg/kg | 12.5 mg/kg |
| ISIS 180281 | 62 | 62 | 59 |
| ISIS 361137 | 59 | 47 | 32 |
| ISIS 361141 | 61 | 49 | 22 |

ISIS 345198 (GTCAAAGGTGCTTTGGTCTG; SEQ ID NO: 307) was chosen for further evaluation in mouse models of diabetes. This compound is perfectly complementary to mouse, rat, human, monkey, rabbit and guinea pig glucocorticoid receptor RNA.

### Example 27

### Additional Studies Showing that Glucocorticoid Receptor Antisense Treatment Reduces Glucocorticoid Receptor Expression In Vivo

The effects of the lead murine glucocorticoid receptor antisense oligonucleotides on glucocorticoid receptor mRNA levels in murine models of type 2 diabetes were examined. After four weeks of systemic administration of glucocorticoid receptor antisense oligonucleotide (ISIS 345198) to ob/ob mice, a dose-dependent reduction of hepatic glucocorticoid receptor mRNA expression was observed. Oligonucleotide was administered s.c. twice a week for four weeks at doses of 6.25 mg/kg, 12.5 mg/kg or 25 mg/kg. Glucocorticoid receptor mRNA levels were reduced by 54%, 67% and 72%, respectively, at these doses, indicating a dose-dependent inhibition of glucocorticoid receptor expression. Control oligonucleotide had no effect on glucocorticoid receptor expression.

### Example 28

### Glucocorticoid Receptor Antisense Treatment with ISIS 345198 Lowers Plasma Glucose Levels in ob/ob Mice

In addition to reducing the level of glucocorticoid receptor mRNA, glucocorticoid receptor antisense oligonucleotide treatment decreased plasma glucose and circulating lipid levels in diabetic models. In saline and control antisense compound-treated ob/ob mice, hyperglycemia continued to worsen throughout the study duration, whereas ISIS 345198-treated animals showed a significant dose-dependent reduction in plasma glucose levels. After 4 weeks of treatment, plasma glucose levels were approximately 225 mg/dL, 250 mg/dL, and 300 mg/dL for mice treated with ISIS 345198 at 25 mg/kg, 12.5 mg/kg and 6.25 mg/kg, respectively. After 4 weeks of saline treatment, mice had plasma glucose levels of approximately 600 mg/dL and mice treated with control oligonucleotide had plasma glucose levels of approximately 490 mg/dL.

Due to the role of glucocorticoid receptor in regulating gluconeogenesis, the effects of glucocorticoid receptor antisense compound on fasted glucose levels were examined. A significant reduction in fasted glucose levels was observed in ob/ob mice (saline 321±16.2 mg/dL vs. ISIS 345198-treated 220±8.3 mg/dL, p<0.05) and db/db mice (saline 320±26.9 mg/dL vs. ISIS 180272-treated 204±24.6 mg/dL, p<0.05). In both models, control antisense compound did not show significant effect on fasted glucose levels. The effects of glucocorticoid receptor antisense inhibition were not accompanied by changes in food intake, body weight or liver glycogen level, measured as described in Desai et al., 2001, Diabetes, 50, 2287-2295 (briefly, liver samples were homogenized in 0.03 N HCl to a final concentration of 0.5 g/ml. The homogenate (100 μl) was mixed with 400 μl of 1.25 N HCl and heated for 1 h at 100° C. Samples were centrifuged at 14,000 rpm, and 10 μl of supernatant was mixed with 1 ml of glucose oxidase reagent (Sigma). After a ten-minute incubation at 37° C., the absorbance was read at 505 nm).

### Example 29

### Effect of Glucocorticoid Receptor Antisense Oligonucleotide on Body Composition, Plasma Resistin, Adiponectin, TNF Alpha, Insulin, and IL-6 Levels in ob/ob Mice Treated with ISIS 345198

Although no change in body weight was observed, densitometric analysis of body composition was performed to accurately measure changes in body fat mass. Body composition analysis was conducted using Lunar X-ray densitometer (GE Medical Systems, Madison, Wis. 53717) in ob/ob mice that were treated with glucocorticoid receptor antisense oligonucleotide (ISIS 345198), 25 mg/kg twice a week for four weeks. Glucocorticoid receptor antisense compound significantly reduced body fat mass after a 4-week treatment period (saline 50.7±0.4 vs. glucocorticoid receptor antisense 45.7±0.5, p<0.05). This reduction was also reflected as a decrease in epididymal fat pad weight (saline 5.09±0.10 grams vs. glucocorticoid receptor antisense 4.3±0.12 grams, p<0.05). Plasma adiponectin, resistin, TNF-alpha and insulin levels were measured by ELISA using kits from Linco Research (St. Charles, Mo. and ALPCO. Windham, N.H.); plasma interleukin-6 (IL-6) levels were measured by ELISA (R&D Systems, Minneapolis, Minn.).

The decrease in adiposity was accompanied by a 20% decrease in plasma resistin levels (saline 22±1.4 ng/ml vs. glucocorticoid receptor antisense 18±0.94 ng/ml), whereas adiponectin levels remained unchanged (saline 8.73±0.14 ug/ml vs. 8.72±0.35 ug/ml). A more robust effect on lowering of TNF alpha (saline 42±2.29 pg/ml vs. glucocorticoid receptor antisense 27±0.86 pg/ml, p<0.05) and insulin levels (saline 43±5.95 ng/ml vs. glucocorticoid receptor antisense 16±1.43 ng/ml, p<0.05) was observed in glucocorticoid receptor antisense-treated mice. Glucocorticoid receptor antisense compound treatment did not result in any significant change in the circulating IL-6 levels (saline 6.27±0.74 pg/ml vs. glucocorticoid receptor antisense-treated 5.37±1.22 pg/ml)

In a separate study, lean, normoglycemic mice received the glucocorticoid receptor antisense compound (ISIS 345198) at 50 mg/kg/week for six weeks. Glucocorticoid receptor inhibition caused a significant reduction in glucocorticoid receptor mRNA expression in the liver (saline 100±5.98 vs. glucocorticoid receptor antisense-treated 24.4±2, p<0.05) and white adipose tissue (saline 100±4 vs. glucocorticoid receptor antisense 31±6, p<0.05) without causing hypoglycemia (24 h fasted levels, saline 147±8 mg/dL vs. glucocorticoid receptor antisense 112±5 mg/dL).

What is claimed is:

1. An antisense compound 12 to 50 nucleobases in length comprising at least one of a modified internucleoside linkage, a high affinity modified sugar or a modified nucleobase, wherein said antisense compound comprises a nucleobase sequence comprising at least 8 contiguous nucleobases complementary to an equal length portion of nucleobases 672-691, nucleobases 679-698, or nucleobases 687-706 of the nucleic acid molecule encoding human glucocorticoid receptor in SEQ ID NO: 4, or an exon:intron region, an intron:exon region, or an intron of the nucleic acid molecule encoding human glucagon receptor in SEQ ID NO: 25, wherein the nucleobase sequence of the antisense compound is at least 90% complementary to SEQ ID NOs: 4 or 25 over the entire length of the antisense compound.

2. The compound of claim 1 which is 15 to 30 nucleobases in length.

3. The compound of claim 1 which is 20 nucleobases in length.

4. The compound of claim 1 having at least 95% complementarity with SEQ ID NOs: 4 or 18 as measured over the entire length of the antisense compound.

5. The compound of claim 1 having at least one 2′-O-methoxyethyl sugar moiety.

6. The compound of claim 1 having at least one phosphorothioate internucleoside linkage.

7. The compound of claim 1 having at least one 5-methylcytosine.

8. The compound of claim 1 that is a pharmaceutically acceptable salt.

9. A pharmaceutical composition comprising the compound of claim 1 and a pharmaceutically acceptable diluent or carrier.

10. The compound of claim 1, comprising at least one high affinity modified sugar moiety selected from the group consisting of a 2′-O-(2-methoxyethyl), a 2′-O-methyl or a locked nucleic acid.

11. The compound of claim 1, wherein the compound is chimeric, comprising deoxynucleotides in a gap region, at least one high affinity modified sugar in each of a first wing region, and a second wing region, said wing regions flanking the gap region on the 5′ end and the 3′ end, respectively, and at least one phosphorothioate modified internucleoside linkage.

12. The compound of claim 11, wherein the gap segment is ten deoxynucleotides in length, the first and second wing segments are each five nucleotides in length and comprise five 2′-O-(2-methoxyethyl) nucleotides, and each internucleoside linkage in the chimeric oligonucleotide is a phosphorothioate.

13. The compound of claim 1, wherein at least a portion of said compound hybridizes with RNA to form an oligonucleotide-RNA duplex.

14. The compound of claim 11, further comprising at least one 5-methylcytosine modified nucleobase.

15. The compound of claim 1 comprising a pharmaceutically acceptable salt.

16. A method of inhibiting expression of glucocorticoid receptor in a cell or tissue comprising contacting said cell or tissue with the antisense compound of claim 1, wherein said antisense compound inhibits the expression of the glucocorticoid receptor.

17. The method of claim 16, wherein the tissue is fat or liver tissue.

18. A method of treating diabetes in an animal comprising administering to said animal a therapeutically or prophylactically effective amount of the antisense compound of claim 1, thereby treating diabetes in the animal.

19. The method of claim 18, wherein said diabetes is Type 2 diabetes.

20. The method of claim 18, wherein basal levels of plasma corticosterone and plasma ACTH in the animal are unaffected by administering the antisense compound.
